(19) United States
(12) Patent Application Publication
(10) Pub. No.: US 2014/0206657 A1
(43) Pub. Date: Jul. 24, 2014

(54) BILE ACID ANALOG TGR5 AGONISTS

(71) Applicant: City of Hope, Duarte, CA (US)

(72) Inventors: Donna Yu, Arcadia, CA (US); Barry Forman, Irvine, CA (US); Wendong Huang, Glendora, CA (US); Kyle Sousa, Pasadena, CA (US)

(73) Assignee: City of Hope, Duarte, CA (US)

(21) Appl. No.: 14/159,995

(22) Filed: Jan. 21, 2014

Related U.S. Application Data
(60) Provisional application No. 61/754,421, filed on Jan. 18, 2013.

(51) Int. Cl. C07J 41/00; C07J 9/00

(52) U.S. Cl. CPC C07J 41/0055; C07J 9/005
USPC 514/177; 514/178; 514/182; 552/553; 552/554; 552/555

(57) ABSTRACT

Provided herein are bile acid analogues and derivatives, methods of synthesizing bile acid analogues and derivatives and their use in treating diabetes and liver disease.

### CROSS-REFERENCES TO RELATED APPLICATIONS

This application claims priority to U.S. Provisional Application No. 61/754,421, filed Jan. 18, 2013.

### BACKGROUND OF THE INVENTION

Diabetes is a burgeoning, worldwide health problem affecting almost twenty-six million people in the United States, with obesity-associated type II diabetes (T2D) accounting for ninety-five percent of all diabetes cases.¹ To date, two bile acids (BAs) receptors have been identified: the nuclear farnesoid X receptor (FXR)³ and the Takeda G-protein-coupled Receptor 5 TGR5.⁴⁻⁵ TGR5 is a cell surface receptor and expressed in monocytes, gall bladder, brown adipose tissue, muscle, liver, and intestine. Its activation by BAs triggers an increase in energy expenditure and attenuates diet-induced obesity.⁶ TGR5 activation by BAs, may regulate glucose homeostasis and insulin sensitivity.

Endogenous BAs are the physiological ligands of TGR5 but are, however, very weak TGR5 ligands in the context of both potency and specificity.⁶ BAs not only activate TGR5, but also trigger activation of the nuclear receptor FXR. Thus, the identification of selective and potent modulators for TGR5 with enhanced efficacy is of crucial and significant value. Provided herein are solutions to these and other problems in the art.

### BRIEF SUMMARY OF THE INVENTION

Disclosed herein, inter alia, are compositions and methods of making bile acid derivatives. Further disclosed herein are methods of treating metabolic disorders using the bile acid derivatives.

In a first aspect is a compound having the formula:

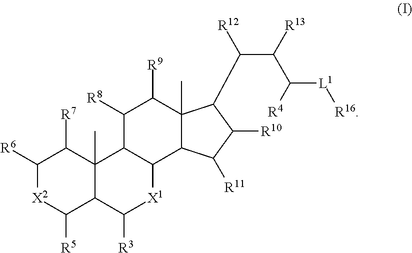

L¹ is —C(O)—, —C(O)O—, —C(O)NH—, or —CH₂—. X¹ is —C(O) or —C(R¹)(R²). X² is —C(O) or —C(R¹⁴)(R¹⁵). R¹ is hydrogen, unsubstituted alkyl, or —OR^{1A}. R² is hydrogen, unsubstituted alkyl, or —OR^{2A}. R³ is hydrogen, unsubstituted alkyl, or —OR^{3A}. R⁴ is hydrogen or unsubstituted alkyl. R⁵ is hydrogen, unsubstituted alkyl, or —OR^{5A}. R⁶ is hydrogen, unsubstituted alkyl, or —OR^{6A}. R⁷ is hydrogen, unsubstituted alkyl, or —OR^{7A}. R⁸ is hydrogen, unsubstituted alkyl, or —OR^{8A}. R⁹ is hydrogen, unsubstituted alkyl, or —OR^{9A}. R¹⁰ is hydrogen, unsubstituted alkyl, or —OR^{10A}. R¹¹ is hydrogen, unsubstituted alkyl, or —OR^{11A}. R¹² is hydrogen, unsubstituted alkyl, or —OR^{12A}. R¹³ is hydrogen, unsubstituted alkyl, or —OR^{13A}. R¹⁴ is hydrogen, unsubstituted alkyl, or —OR^{14A}. R¹⁵ is hydrogen, unsubstituted alkyl, or —OR^{15A}. R¹⁶ is hydrogen, halogen, —N₃, —CF₃, —CCl₃, —CBr₃, —CI₃, —CN, —CHO, —OR^{16A}, —NHR^{16A}, —COOH, —CONH₂, —NO₂, —SH, —SO₂Cl, —SO₃H, —SO₄H, —SO₂NH₂, —NHNH₂, —ONH₂, —NHC(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, or a carboxylate protecting group. R^{1A}, R^{2A}, R^{3A}, R^{5A}, R^{6A}, R^{7A}, R^{8A}, R^{9A}, R^{10A}, R^{11A}, R^{12A}, R^{13A}, R^{14A}, R^{15A} are independently hydrogen, unsubstituted alkyl, or an alcohol protecting group. R^{16A} is hydrogen, halogen, —N₃, —CF₃, —CCl₃, —CBr₃, —CI₃, —CN, —CHO, —OH, —NH₂, —COOH, —CONH₂, —NO₂, —SH, —SO₂Cl, —SO₃H, —SO₄H, —SO₂NH₂, —NHNH₂, —ONH₂, —NHC(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, an alcohol protecting group, or a carboxylate protecting group. If X¹ or X² is —C(H)OH and R¹R², R³, and R⁴ are hydrogen, then -L¹-R¹⁶ is not —C(O)OH. If X and X² are —C(H)OH, R³ is α-ethyl and R⁴ is hydrogen, then -L¹-R¹⁶ is not —C(O)OH.

In another aspect, a pharmaceutical composition is provided including a pharmaceutically acceptable excipient and a compound as provided herein (e.g. formula (I), (II), (III), (IV), (VI), (VII) or a compound of Table 1), including embodiments thereof.

In another aspect is a method of synthesizing a compound of Formula (I). The method includes contacting a compound of Formula (II) with an alkylating agent in the presence of a sterically hindered base and contacting said compound of Formula (II) with a carboxylate deprotecting agent, thereby synthesizing a compound of Formula (I)

In another aspect is a method of treating or preventing diabetes, obesity, insulin resistance, or liver disease in a subject in need thereof. The method includes ministering to said subject a therapeutically effective amount of a compound as provided herein (e.g. formula (I), (II), (III), (IV), (VI), (VII) or a compound of Table 1), including embodiments thereof.

In another aspect is a method of treating or preventing cancer. The method includes administering to said subject in need thereof, a therapeutically effective amount of a compound as provided herein (e.g. formula (I), (II), (III), (IV), (VI), (VII) or a compound of Table 1), including embodiments thereof.

### BRIEF DESCRIPTION OF THE DRAWINGS

FIG. 1: Chemical structures of some natural bile acids and previously disclosed TGR5 synthetic agonists.

FIG. 2: Design of a system exploiting the base-promoted electrophlic reaction for stereoselective α-functionalization.

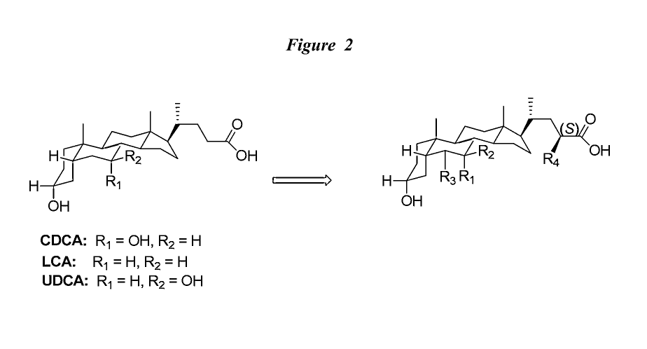

FIG. 3: Syntheses of 23(S)-methyl-CDCA, 23(S)-methyl-UDCA, and 23-methyl-LCA.

FIG. 4: Highly stereoselective synthesis of 6α-ethyl-LCA and 23(S)-6α-ethyl-LCA.

FIG. 5: Highly stereoselective synthesis of 6α-ethyl-UDCA, 23(S)-methyl-6α-ethyl-UDCA.

FIG. 6: Proposed mechanism for stereoselective methylation: A) the complex of KHMDS deprotonating CDCA ester wherein alkylation on the si face of the incipient enolate would give the desired (S) methyl at C23; B) Enolates of 10a, 10b, and 17, stabilized by complexation through a potassium cation to oxygens associated with C7—the structures were energy-minimized by molecular mechanics; c) MMFF-minimized structures showing the C11 steric hindrance of the re face of the α-enolate carbon (C23) for the complexes of 10a, 10b, and 17.

FIG. 7: Alkylation of LCA at the C3 position.

FIG. 8A-D: TGR5 luciferase reporting activities of alkyl-substituted bile acids; all of the tested 23(S)-alkyl-substituted bile acid derivatives displayed markedly enhanced TGR5 specificity and potency relative to their native bile acids, wherein FIG. 8B shows that compound 23(S)-Me-LCA is a better agonist in terms of potency and selectivity for TGR5 as compared with LCA and FIG. 8C shows that 23(S)-Me-UDCA is a better agonist than UDCA.

FIG. 9: 23(S)-methyl substitution of bile acids increases their potency for TGR5, but not FXR, in vitro: HEK293 cells that overexpressed TGR5 were treated with increasing doses of native and 23(S)-methylated bile acids for 16 h in quadruplicate and normalized with β-galactosidase and TGR5-reporting activity was evaluated by luciferase assay.

FIG. 10 (A-B): An example of a hydrogen bonding network involving the acid tail of 23(S)-methyl-LCA, wherein FIG. 10A shows that the methyl sits in a pocket of aromatic residues in the (S) isomer but causes a steric clash in the (R) isomer as shown in FIG. 10B.

FIG. 11: Placement of the 6α-ethyl in 6α-ethyl-23(S)-methyl-LCA, as well as a view of the Tyr 3.29-Glu 5.42-Tyr 6.51 hydrogen bonding network.

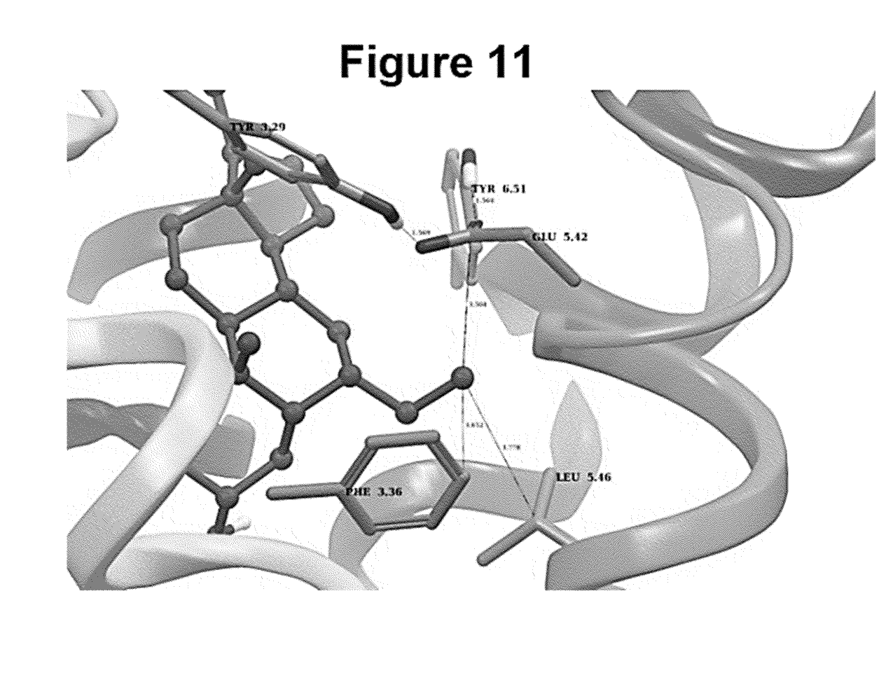

FIG. 12: Synthesis of 23(S)-methyl-3,7-diketo-5,3-cholan-24-oic acid.

FIG. 13: Docking of LCA to TGR5.

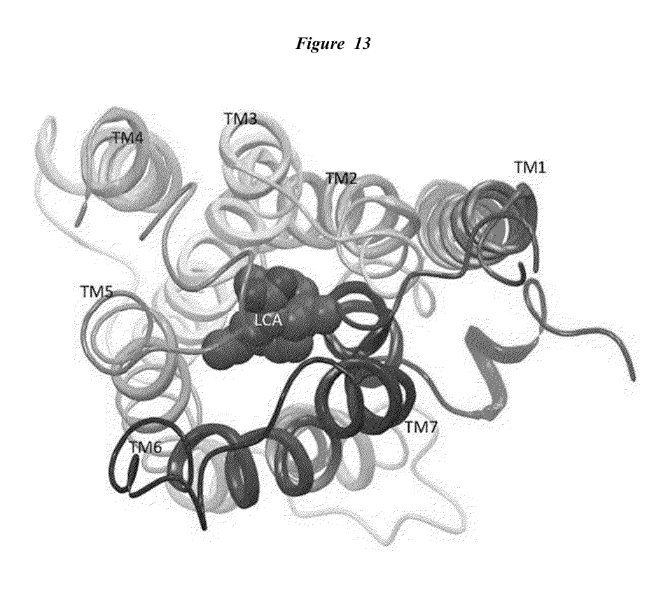

FIG. 14: Placement of the 3α-OH on 6α-ethyl-23(S)-methyl-LCA.

FIG. 15: Proposed mechanistic pathways for the stereospecific MPV reduction with aluminum isopropoxide.

### DETAILED DESCRIPTION OF THE INVENTION

### I. Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals, having the number of carbon atoms designated (i.e., C₁-C₁₀ means one to ten carbons). An alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—).

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred in the present invention. An alkylene is au uncyclized chain. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable straight or branched chain, or combinations thereof, including at least one carbon atom and at least one heteroatom selected from the group consisting of O, N, P, Si, and S, and wherein the nitrogen and sulfur atoms may optionally be oxidized, and the nitrogen heteroatom may optionally be quaternized. A heteroalkyl is an uncyclized chain. The heteroatom(s) O, N, P, S, B, As, and Si may be placed at any interior position of the heteroalkyl group or at the position at which the alkyl group is attached to the remainder of the molecule. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. A heteroalkylene is an uncyclized chain. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Additionally, for heterocycloalkyl, a heteroatom can occupy the position at which the heterocycle is attached to the remainder of the molecule. A cycloalkyl or heteroalkyl is not aromatic. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be a —O— bonded to a ring heteroatom nitrogen.

A “fused ring aryl-heterocycloalkyl” is an aryl fused to a heterocycloalkyl. A “fused ring heteroaryl-heterocycloalkyl” is a heteroaryl fused to a heterocycloalkyl. A “fused ring heterocycloalkyl-cycloalkyl” is a heterocycloalkyl fused to a cycloalkyl. A “fused ring heterocycloalkyl-heterocycloalkyl” is a heterocycloalkyl fused to another heterocycloalkyl. Fused ring aryl-heterocycloalkyl, fused ring heteroaryl-heterocycloalkyl, fused ring heterocycloalkyl-cycloalkyl, or fused ring heterocycloalkyl-heterocycloalkyl may each independently be unsubstituted or substituted with one or more of the substitutents described herein. Fused ring aryl-heterocycloalkyl, fused ring heteroaryl-heterocycloalkyl, fused ring heterocycloalkyl-cycloalkyl, or fused ring heterocycloalkyl-heterocycloalkyl may each independently be named according to the size of each of the fused rings. Thus, for example, 6,5 aryl-heterocycloalkyl fused ring describes a 6 membered aryl moiety fused to a 5 membered heterocycloalkyl. Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g. substitutents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g. all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The term “oxo,” as used herein, means an oxygen that is double bonded to a carbon atom.

Each of the above terms (e.g., “alkyl,” “heteroalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C═(O)NR″NR′″R″″, —CN, —NO₂, —NR′SO₂R″, —NR′C═(O)R″, —NR′C(O)—OR″, —NR′OR″, in a number ranging from zero to (2m′+1), where m′ is the total number of carbon atoms in such radical. R′, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound of the invention includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC (O)NR′R″, —NR″C(O)R′, —NR′—C(O)NR″R′″, —NR″C(O)₂R′, —NR—C(NR′R″R′″)═NR″″, —NR—C(NR″R″)═NR′″, —S(O)R′, —S(O)₂R′, —S(O)₂NR′R″, —NRSO₂R′, —NR′NR″R′″, —ONR′R″, —NR′C═(O)NR″NR′″R″″, —CN, —NO₂, —R′, —N₃, —CH(Ph)₂, fluoro(C₁-C₄)alkoxy, and fluoro(C₁-C₄)alkyl, —NR′SO₂R″, —NR′C═(O)R″, —NR′C(O)—OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound of the invention includes more than one R group, for example, each of the R groups is independently selected as are each R′, R″, R′″, and R″″ groups when more than one of these groups is present.

Substituents for rings (e.g. cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g. a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R″R′″)_{d}—, where s and d are independently integers of from 0 to 3, and X′ is —O—, —NR′—, —S—, —S(O)—, —S(O)₂—, or —S(O)₂NR′—. The substituents R′, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include, oxygen (O), nitrogen (N), sulfur (S), phosphorus (P), Boron (B), Arsenic (As), and silicon (Si).

A “substituent group,” as used herein, means a group selected from the following moieties:
(A) oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and
(B) alkyl, heteroalkyl, cycloalkyl, heterocycloalkyl, aryl, and heteroaryl, substituted with at least one substituent selected from:
   (i) oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and
   (ii) alkyl, heteroalkyl, cycloalkyl, heterocycloalkyl, aryl, and heteroaryl, substituted with at least one substituent selected from:
      (a) oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and
      (b) alkyl, heteroalkyl, cycloalkyl, heterocycloalkyl, aryl, or heteroaryl, substituted with at least one substituent selected from: oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, and unsubstituted heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, and/or each substituted or unsubstituted heterocycloalkyl is a substituted or unsubstituted 3 to 8 membered heterocycloalkyl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, and/or each substituted or unsubstituted heterocycloalkylene is a substituted or unsubstituted 3 to 8 membered heterocycloalkylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, and/or each substituted or unsubstituted heterocycloalkyl is a substituted or unsubstituted 3 to 7 membered heterocycloalkyl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, and/or each substituted or unsubstituted heterocycloalkylene is a substituted or unsubstituted 3 to 7 membered heterocycloalkylene.

Certain compounds of the present invention possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present invention. The compounds of the present invention do not include those which are known in art to be too unstable to synthesize and/or isolate. The present invention is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this invention may exist in tautomeric forms, all such tautomeric forms of the compounds being within the scope of the invention.

The term “silyl ether” as used herein, refers to a chemical compound containing a silicon atom covalently bonded to an alkoxy group generally having the structure R^{w}R^{x}R^{y}Si—O—R^{z}, wherein R^{w}, R^{x}, R^{y}, and R^{z} are independently alkyl or aryl groups.

The term “pharmaceutically acceptable salts” is meant to include salts of the active compounds that are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

Thus, the compounds of the present invention may exist as salts, such as with pharmaceutically acceptable acids. The present invention includes such salts. Examples of such salts include hydrochlorides, hydrobromides, sulfates, methanesulfonates, nitrates, maleates, acetates, citrates, fumarates, tartrates (e.g., (+)-tartrates, (−)-tartrates, or mixtures thereof including racemic mixtures), succinates, benzoates, and salts with amino acids such as glutamic acid. These salts may be prepared by methods known to those skilled in the art.

The neutral forms of the compounds are preferably regenerated by contacting the salt with a base or acid and isolating the parent compound in the conventional manner. The parent form of the compound differs from the various salt forms in certain physical properties, such as solubility in polar solvents.

In addition to salt forms, the present invention provides compounds, which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

As used herein, the term “salt” refers to acid or base salts of the compounds used in the methods of the present invention. Illustrative examples of salts include mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts. The term salt also refers to formation of a salt between two compounds.

Certain compounds of the present invention possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present invention. The compounds of the present invention do not include those which are known in art to be too unstable to synthesize and/or isolate. The present invention is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

It will be apparent to one skilled in the art that certain compounds of this invention may exist in tautomeric forms, all such tautomeric forms of the compounds being within the scope of the invention.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the invention.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by 13C- or 14C-enriched carbon are within the scope of this invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present invention, whether radioactive or not, are encompassed within the scope of the present invention.

The symbol “ [symbol] ” denotes the point of attachment of a chemical moiety to the remainder of a molecule or chemical formula.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls. Moreover, where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R¹³ substituents are present, each R¹³ substituent may be distinguished as R^{13A}, R^{13B}, R^{13C}, R^{13D}, etc., wherein each of R^{13A}, R^{13B}, R^{13C}, R^{13D}, etc. is defined within the scope of the definition of R¹³ and optionally differently.

Description of compounds of the present invention is limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule viaring heteroatom in compliance with principles of chemical bonding known to those skilled in the art thereby avoiding inherently unstable compounds.

The terms “treating” or “treatment” refers to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, include prevention of an injury, pathology, condition, or disease.

A “therapeutically effective amount” or “effective amount” is an amount sufficient for a compound to accomplish a stated purpose relative to the absence of the compound (e.g. achieve the effect for which it is administered, treat a disease, reduce enzyme activity, increase enzyme activity, reduce a signaling pathway, or reduce one or more symptoms of a disease or condition). A therapeutically effective amount is an amount sufficient to contribute to the treatment, prevention, or reduction of a symptom or symptoms of a disease. A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

“Patient” or “subject in need thereof” refers to a living organism suffering from or prone to a condition that can be treated by administration of a pharmaceutical composition as provided herein. Non-limiting examples include humans, other mammals (e.g. mice, rats, dogs, monkeys, cows, goats, sheep) and other non-mammalian animals. In some embodiments, a patient or subject in need thereof is a human with a disease or condition.

The term “diabetes” as used herein refers to onset and inducement of diabetes mellitus in any manner and includes type 1, type 2, gestational, steroid-induced, HIV treatment induced and autoimmune diabetes. Diabetes is recognized as a complex, chronic disease in which 60% to 70% of all case fatalities among diabetic patients are a result of cardiovascular complications. Diabetes is not only considered a coronary heart disease risk equivalent but is also identified as an independent predictor of adverse events, including recurrent myocardial infarction, congestive heart failure, and death following a cardiovascular incident. The adoption of tighter glucose control and aggressive treatment for cardiovascular risk factors would be expected to reduce the risk of coronary heart disease complications and improve overall survival among diabetic patients. Yet, diabetic patients are two to three times more likely to experience an acute myocardial infarction than non-diabetic patients, and diabetic patients live eight to thirteen years less than non-diabetic patients.

As used herein, the term “liver disease” refers to any symptoms related to liver dysfunction including physical signs and symptoms related to digestive problems, blood sugar disorders, immune disorders, and abnormal fat absorption and metabolism. Liver disease as used herein refers to all types of liver dysfunction including hepatitis, alcoholic liver disease, fatty liver disease, non-alcoholic fatty liver disease, inflammatory liver disease, cirrhosis, hereditary diseases, and cancers associated with the liver.

As used herein, the term “cancer” refers to all types of cancer, neoplasm, benign or malignant tumors found in mammals, including. Exemplary cancers include liver, colon, kidney, and stomach cancers. Additional examples include lung, non-small cell lung, brain, breast, pancreas, prostate, ovary, sarcoma, melanoma, cervix, head & neck, and uterus cancers, as well as leukemia, carcinomas and sarcomas, mesothelioma, metastatic bone cancer, Medulloblastoma, Hodgkin's Disease, Non-Hodgkin's Lymphoma, multiple myeloma, neuroblastoma, rhabdomyosarcoma, primary thrombocytosis, primary macroglobulinemia, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, and neoplasms of the endocrine and exocrine pancreas. In embodiments, the cancer may be liver cancer.

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity of a protein in the absence of a compound as described herein (including embodiments and examples).

“Contacting” is used in accordance with its plain ordinary meaning and refers to the process of allowing at least two distinct species (e.g. chemical compounds including biomolecules or cells) to become sufficiently proximal to react, interact or physically touch. It should be appreciated; however, the resulting reaction product can be produced directly from a reaction between the added reagents or from an intermediate from one or more of the added reagents which can be produced in the reaction mixture.

The term “contacting” may include allowing two species to react, interact, or physically touch, wherein the two species may be a compound as described herein and a protein or enzyme. In some embodiments contacting includes allowing a compound described herein to interact with a protein or enzyme that is involved in a signaling pathway.

The term “modulator” refers to a composition that increases or decreases the level of a target molecule or the function of a target molecule or the physical state of the target of the molecule.

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

As used herein, the term “administering” means oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies. The compounds of the invention can be administered alone or can be coadministered to the patient. Coadministration is meant to include simultaneous or sequential administration of the compounds individually or in combination (more than one compound). Thus, the preparations can also be combined, when desired, with other active substances (e.g. to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols.

The term “sterically hindered base” refers to a chemical base that reacts with a carbon atom within a separate molecule in a preferred stereochemical orientation due to spatial contraints caused by its attached chemical moieties, which are typically bulky chemically moieties. Sterically hindered bases useful in the present methods include: tBuO; PhO; MeO; EtO; bis(trimethylsilyl)amide (HMDS); 2,2,6,6-tetramethylpiperidine (TMP); 1,8-Diazabicyclo[5.4.0]undec-7-ene (DBU); 1,4-Diazabicyclo[2.2.2]octane (Dabco); 1,5-Diazabicyclo[4.3.0]non-5-ene (DBN); N,N-dichlorohexylmethylamine; N,N-diisopropyl-2-ethylbutylamine; 2,6-di-tert-butyl-4-methylpyridine; 7-Methyl-1,5,7-triazabicyclo(4.4.0)dec-5-ene (MTBD); 3,3,6,9,9-Pentamethyl-2,10-diazabicyclo-(4.4.0)dec-1-ene (PMDBD); 1,2,2,6,6-Pentamethylpiperidine (PMP); 1,5,7-Triazabicyclo(4.4.0)dec-5-ene (TBD); or tri-tert-butylpyridine. Bases may be complexed with M⁺¹ charged metals such as K, Na, or Li.

As used herein, the term “carboxylate protecting group” is a monovalent chemical moiety covalently bound to a monovalent carboxylate moiety oxygen atom that functions to prevent the carboxylate moiety from reacting with reagents used in the chemical synthetic methods described herein (commonly referred to as “protecting” the carboxylate group) and may be removed under conditions that do not degrade the molecule of which the carboxylate moiety forms a part (commonly referred to as “deprotecting” the carboxylate group) thereby yielding a free carboxylic acid. A carboxylate protecting group can be acid labile, base labile, or can be labile in the presence of other reagents. Carboyxlate protecting groups include but are not limited to: methyl ester; t-butyl ester; 2,2,2-trichloroethyl ester; 4-nitrobenzyl ester; cyanoethyl ester; 4-methyl-2,6,7-trioxabicyclo[2.2.2]octane; iminoethers; or lactones.

As used herein, the term “carboxylate deprotecting agent” is a chemical compound or element that functions to remove a carboxylate protecting group, thereby yielding a free carboxylic acid. Carboxylate deprotecting agents useful in the present methods include: LiOH, diethyl amine, triethyl amine, piperidine, tetrabutylammonium hydroxide, fluoride ion, hydrogenation, or sodium.

As used herein, the term “alcohol protecting group” is a monovalent chemical moiety covalently bound to a monovalent alcohol oxygen atom that functions to prevent the alcohol moiety from reacting with reagents used in the chemical synthetic methods described herein (commonly referred to as “protecting” the alcohol group) and may be removed under conditions that do not degrade the molecule of which the alcohol moiety forms a part (commonly referred to as “deprotecting” the alcohol group) thereby yielding a free hydroxyl. An alcohol protecting group can be acid labile, base labile, or labile in the presence of other reagents. Alcohol protecting groups include but are not limited to: benzyl (Bn); p-methoxybenzyl (PMB); dimethoxybenzyl (DMB), allyl; allyl carbonate, trityl (Trt); p-methoxyphenyl (PMP); tetrahydropyranyl (THP); methoxymethyl (MOM); 1-ethoxyethyl (EE); 2-methoxy-2-propyl (MOP); 2,2,2-trichloroethoxymethyl; 2-methoxyethoxymethyl (MEM); 2-trimethylsilylethoxymethyl (SEM); methylthiomethyl (MTM), trimethylsilyl (TMS); triethylsilyl (TES); triisopropylsilyl (TIPS); triphenylsilyl (TPS); triphenylmethyl (Tr), t-butyldimethylsilyl (TBDMS); t-butyldiphenylsilyl (TBDPS); acetyl (Ac); benzyloxy (Bz); 2,2,2-trichloroethyl carbonate (Troc); or 2-trimethylsilylthehyl carbonate.

As used herein, the term “alcohol deprotecting agent” is a chemical compound or element that functions to remove an alcohol protecting group, thereby yielding a free hydroxyl. Alcohol deprotecting agents useful in the present methods include: zinc bromide, magnesium bromide, titanium tetrachloride, dimethylboron bromide, trimethylsilyl iodide, silver (Ag+) salts, mercury (Hg+) salts, zinc, samarium diiodide, sodium amalgam, trifluoroacetic acid, hydrofluoric acid, hydrochloric acid, hydrogenation, (TBAF) tetra-n-butylammonium fluoride, boron trifluoride, or silicon tetrafluoride.

The term “reducing agent” is a chemical compound or element that donates electrons to another chemical compound in an oxidation-reduction reaction. Reducing agents are typically used to add a hydrogen to a molecule.

The term “oxidizing agent” is a chemical compound or element that accepts or gains electrons from another chemical compound in an oxidation-reduction reaction.

The term “polar aprotic solvent” refers to a chemical compound used as a solvent having a dipole moment, and therefore polarity, but lacking an ability to hydrogen bond through —OH or —NH bonds with itself or other compounds. Such solvents include but are not limited to: hexamethylphosphoramide (HMPA), hexamethylphosphorous triamide (HMPT), N,N-dimethylformamide (DMF), dimethyl sulfoxide (DMSO), acetonitrile (MeCN), dioxane, acetone, tetrahydrofuran (THF), N-methylpyrrolidone (NMP), N,N′-dimethyl-N,N′-trimethyleneurea (DMPU), or tetra-alkyl ureas.

A “coupling reagent” as used herein is a chemical compound that forms an activated ester used in forming amide bonds, such as non-racemized amide bonds. Coupling reagents useful in the present methods include but are not limited to: EEDQ (N-ethoxy-2-ethoxy-1,2-dihydroquinoline); BOP (Benzotriazole-1-yl-oxy-tris-(dimethylamino)-phosphoniumhexafluorophospate); PyBOP (Benzotriazole-1-yl-oxy-tris-pyrrolidino-phosphonium hexafluorophosphate); DMAP (4-dimethylaminopyridine); HATU (2-(O-(7-azabenzotriazol-1-yl)-1,1,3,3-tetramethyluronium hexafluorophosphate); HBTU (2-(1H-benzotriazole-11-yl)-1,1,3,3-tetramethylaminium hexafluorophosphate); HCTU (2-(6-chloro-1H-benzotriazole-1-yl)-1,1,3,3-tetramethylaminium hexafluorophosphate; or HObt (N-hydroxybenzotriazole); TOTU (O-((ethoxycarbonyl)cyanomethylene amino)-N,N,N′N′-tetramethyluronium tetrafluoroborate); DCC (dicyclohexyl carbodiimide); DIC (diisopropyl carbodiimide); EDC (1-ethyl-3-(3′-dimethylamino)carbodiimide); (DMTMM) 4-(4,6-dimethoxy-(1,3,5)triazine-2-yl)-4-methyl-morpholinium chloride; 1-Chloro-4,6-dimethoxy-1,3,5-triazine.

### II. Compositions

In one aspect is a compound having formula (I), wherein

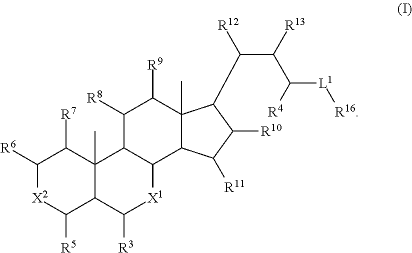

L¹ is —C(O)—, —C(O)O—, —C(O)NH—, or —CH₂—. X¹ is —C(O) or —C(R¹)(R²). X² is —C(O) or —C(R¹⁴)(R¹⁵). R¹ is hydrogen, halogen, —N₃, —CF₃, —CCl₃, —CBr₃, —CI₃, —CN, —CHO, —OR^{1A}, —NH₂, —COOH, —CONH₂, —NO₂, —SH, —SO₂Cl, —SO₃H, —SO₄H, —SO₂NH₂, —NHNH₂, —ONH₂, —NHC(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R² is hydrogen, halogen, —N₃, —CF₃, —CCl₃, —CBr₃, —CI₃, —CN, —CHO, —OR^{2A}, —NH₂, —COOH, —CONH₂, —NO₂, —SH, —SO₂Cl, —SO₃H, —SO₄H, —SO₂NH₂, —NHNH₂, —ONH₂, —NHC(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R³ is hydrogen, halogen, —N₃, —CF₃, —CCl₃, —CBr₃, —CI₃, —CN, —CHO, —OR^{3A}, —NH₂, —COOH, —CONH₂, —NO₂, —SH, —SO₂Cl, —SO₃H, —SO₄H, —SO₂NH₂, —NHNH₂, —ONH₂, —NHC(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R⁴ is hydrogen, halogen, —N₃, —CF₃, —CCl₃, —CBr₃, —CI₃, —CN, —CHO, —OR^{4A}, —NH₂, —COOH, —CONH₂, —NO₂, —SH, —SO₂Cl, —SO₃H, —SO₄H, —SO₂NH₂, —NHNH₂, —ONH₂, —NHC(O)NHNH₂, substituted or unsubstituted alkyl, or substituted or unsubstituted heteroalkyl. R⁵ is hydrogen, halogen, —N₃, —CF₃, —CCl₃, —CBr₃, —CI₃, —CN, —CHO, —OR^{5A}, —NH₂, —COOH, —CONH₂, —NO₂, —SH, —SO₂Cl, —SO₃H, —SO₄H, —SO₂NH₂, —NHNH₂, —ONH₂, —NHC(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R⁶ is hydrogen, halogen, —N₃, —CF₃, —CCl₃, —CBr₃, —CI₃, —CN, —CHO, —OR^{6A}, —NH₂, —COOH, —CONH₂, —NO₂, —SH, —SO₂Cl, —SO₃H, —SO₄H, —SO₂NH₂, —NHNH₂, —ONH₂, —NHC(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R⁷ is hydrogen, halogen, —N₃, —CF₃, —CCl₃, —CBr₃, —CI₃, —CN, —CHO, —OR^{7A}, —NH₂, —COOH, —CONH₂, —NO₂, —SH, —SO₂Cl, —SO₃H, —SO₄H, —SO₂NH₂, —NHNH₂, —ONH₂, —NHC(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R⁸ is hydrogen, halogen, —N₃, —CF₃, —CCl₃, —CBr₃, —CI₃, —CN, —CHO, —OR^{8A}, —NH₂, —COOH, —CONH₂, —NO₂, —SH, —SO₂Cl, —SO₃H, —SO₄H, —SO₂NH₂, —NHNH₂, —ONH₂, —NHC(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R⁹ hydrogen, halogen, —N₃, —CF₃, —CCl₃, —CBr₃, —CI₃, —CN, —CHO, —OR^{9A}, —NH₂, —COOH, —CONH₂, —NO₂, —SH, —SO₂Cl, —SO₃H, —SO₄H, —SO₂NH₂, —NHNH₂, —ONH₂, —NHC(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R¹⁰ hydrogen, halogen, —N₃, —CF₃, —CCl₃, —CBr₃, —CI₃, —CN, —CHO, —OR^{10A}, —NH₂, —COOH, —CONH₂, —NO₂, —SH, —SO₂Cl, —SO₃H, —SO₄H, —SO₂NH₂, —NHNH₂, —ONH₂, —NHC(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R¹¹ hydrogen, halogen, —N₃, —CF₃, —CCl₃, —CBr₃, —CI₃, —CN, —CHO, —OR^{11A}, —NH₂, —COOH, —CONH₂, —NO₂, —SH, —SO₂Cl, —SO₃H, —SO₄H, —SO₂NH₂, —NHNH₂, —ONH₂, —NHC(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R¹² hydrogen, halogen, —N₃, —CF₃, —CCl₃, —CBr₃, —CI₃, —CN, —CHO, —OR^{12A}, —NH₂, —COOH, —CONH₂, —NO₂, —SH, —SO₂Cl, —SO₃H, —SO₄H, —SO₂NH₂, —NHNH₂, —ONH₂, —NHC(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R¹³ hydrogen, halogen, —N₃, —CF₃, —CCl₃, —CBr₃, —CI₃, —CN, —CHO, —OR^{13A}, —NH₂, —COOH, —CONH₂, —NO₂, —SH, —SO₂Cl, —SO₃H, —SO₄H, —SO₂NH₂, —NHNH₂, —ONH₂, —NHC(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R¹⁴ hydrogen, halogen, —N₃, —CF₃, —CCl₃, —CBr₃, —CI₃, —CN, —CHO, —OR^{14A}, —NH₂, —COOH, —CONH₂, —NO₂, —SH, —SO₂Cl, —SO₃H, —SO₄H, —SO₂NH₂, —NHNH₂, —ONH₂, —NHC(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R⁵ hydrogen, halogen, —N₃, —CF₃, —CCl₃, —CBr₃, —CI₃, —CN, —CHO, —OR^{15A}, —NH₂, —COOH, —CONH₂, —NO₂, —SH, —SO₂Cl, —SO₃H, —SO₄H, —SO₂NH₂, —NHNH₂, —ONH₂, —NHC(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R¹⁶ hydrogen, halogen, —N₃, —CF₃, —CCl₃, —CBr₃, —CI₃, —CN, —CHO, —OR^{16A}, —NHR^{16A}, —COOH, —CONH₂, —NO₂, —SH, —SO₂Cl, —SO₃H, —SO₄H, —SO₂NH₂, —NHNH₂, —ONH₂, —NHC(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R^{1A}, R^{2A}, R^{3A}, R^{5A}, R^{6A}, R^{7A}, R^{8A}, R^{9A}, R^{10A}, R^{11A}, R^{12A}, R^{13A}, R^{14A}, R^{15A} are independently hydrogen, unsubstituted alkyl, or an alcohol protecting group. R^{16A} is hydrogen, halogen, —N₃, —CF₃, —CCl₃, —CBr₃, —CI₃, —CN, —CHO, —OH, —NH₂, —COOH, —CONH₂, —NO₂, —SH, —SO₂Cl, —SO₃H, —SO₄H, —SO₂NH₂, —NHNH₂, —ONH₂, —NHC(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, an alcohol protecting group, or a carboxylate protecting group. If X¹ or X² is —C(H)OH— and R¹, R², R³, and R⁴ are hydrogen, then -L¹-R¹⁶ is not —C(O)OH. If X¹ and X² are —C(H)OH—, R³ is α-ethyl and R⁴ is hydrogen, then -L¹-R¹⁶ is not —C(O)OH.

In another aspect is a compound having a formula:

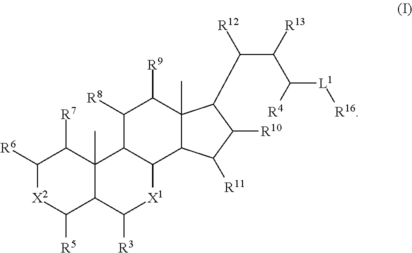

L¹ is —C(O)—, —C(O)O—, —C(O)NH—, or —CH₂—. X¹ is —C(O) or —C(R¹)(R²). X² is —C(O) or —C(R¹⁴)(R¹⁵). R¹ is hydrogen, unsubstituted alkyl, or —OR^{1A}. R² is hydrogen, unsubstituted alkyl, or —OR^{2A}. R³ is hydrogen, unsubstituted alkyl, or —OR^{3A}. R⁴ is hydrogen or unsubstituted alkyl. R⁵ is hydrogen, unsubstituted alkyl, or —OR^{5A}. R⁶ is hydrogen, unsubstituted alkyl, or —OR^{6A}. R⁷ is hydrogen, unsubstituted alkyl, or —OR^{7A}. R⁸ is hydrogen, unsubstituted alkyl, or —OR^{8A}. R⁹ is hydrogen, unsubstituted alkyl, or —OR^{9A}. R¹⁰ is hydrogen, unsubstituted alkyl, or —OR^{10A}. R¹¹ is hydrogen, unsubstituted alkyl, or —OR^{11A}. R¹² is hydrogen, unsubstituted alkyl, or —OR^{12A}. R³ is hydrogen, unsubstituted alkyl, or —OR^{13A}. R¹⁴ is hydrogen, unsubstituted alkyl, or —OR^{14A}. R¹⁵ is hydrogen, unsubstituted alkyl, or —OR^{15A}. R¹⁶ is hydrogen, halogen, —N₃, —CF₃, —CCl₃, —CBr₃, —CI₃, —CN, —CHO, —OR^{16A}, —NHR^{16A}, —COOH, —CONH₂, —NO₂, —SH, —SO₂Cl, —SO₃H, —SO₄H, —SO₂NH₂, —NHNH₂, —ONH₂, —NHC(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, or a carboxylate protecting group. R^{1A}, R^{2A}, R^{3A}, R^{5A}, R^{6A}, R^{7A}, R^{8A} R^{9A}, R^{10A}, R^{11A}, R^{12A}, R^{13A}, R^{14A}, R^{15A} are independently hydrogen, unsubstituted alkyl, or an alcohol protecting group. R^{16A} is hydrogen, halogen, —N₃, —CF₃, —CCl₃, —CBr₃, —CI₃, —CN, —CHO, —OH, —NH₂, —COOH, —CONH₂, —NO₂, —SH, —SO₂Cl, —SO₃H, —SO₄H, —SO₂NH₂, —NHNH₂, —ONH₂, —NHC(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, an alcohol protecting group, or a carboxylate protecting group. If X¹ or X² is —C(H)OH— and R¹, R², R³, and R⁴ are hydrogen, then -L¹-R¹⁶ is not —C(O)OH. If X¹ and X² are —C(H)OH—, R³ is α-ethyl and R⁴ is hydrogen, then -L¹-R¹⁶ is not —C(O)OH.

In embodiments, if X¹ and X² are —C(H)OH and R³ is unsubstituted C₁-C₅ alkyl, then -L¹-R¹⁶ is not —C(O)OH. In embodiments, if X¹ and X² are —C(H)OH and R³ is unsubstituted ethyl, then -L¹-R¹⁶ is not —C(O)OH. In embodiments, if X¹ and X² are —C(H)OH and R⁴ is unsubstituted C₁-C₅ alkyl, then -L¹-R¹⁶ is not —C(O)OH. In embodiments, if X¹ and X² are —C(H)OH and R⁴ is methyl, then -L¹-R¹⁶ is not —C(O)OH. In embodiments, if X¹ and X² are —C(H)OH and R³ and R⁴ are unsubstituted C₁-C₅ alkyl, then -L¹-R¹⁶ is not —C(O)OH. In embodiments, if X¹ and X² are —C(H)OH, and R³ and R⁴ are hydrogen, then -L¹-R¹⁶ is not —C(O)OH. In embodiments, if X¹ and X² are —C(O), and R³ and R⁴ are hydrogen, then -L¹-R¹⁶ is not —C(O)OH. In embodiments, if X¹ and X² are —C(O), and R³ and R⁴ are unsubstituted C₁-C₅ alkyl, then -L¹-R¹⁶ is not —C(O)OH.

R⁵, R⁶, R⁷, R⁸, R⁹, R^{°}, R″, R¹², and R¹³ may independently be hydrogen or unsubstituted alkyl. R⁵, R⁶, R⁷, R⁸, R⁹, R¹⁰, R¹¹, R¹², and R¹³ may independently be hydrogen. R⁵, R⁶, R⁷, R⁸, R⁹, R¹⁰, R¹¹, R¹², and R¹³ may independently unsubstituted alkyl. R⁵, R⁶, R⁷, R⁸, R⁹, R¹⁰, R¹¹, R¹², and R¹³ may independently be C₁-C₂₀ unsubstituted alkyl. R⁵, R⁶, R⁷, R⁸, R⁹, R¹⁰, R¹¹, R¹², and R¹³ may independently be C₁-C₁₀ unsubstituted alkyl. R⁵, R⁶, R⁷, R⁸, R⁹, R¹⁰, R¹¹, R¹², and R¹³ may independently be C₁-C₅ unsubstituted alkyl. R⁵, R⁶, R⁷, R⁸, R⁹, R¹⁰, R¹¹, R¹², and R¹³ may independently be unsubstituted methyl or unsubstituted ethyl. R¹² may be methyl. R¹² may be attached to a chiral carbon having (R) stereochemistry.

R⁵, R⁶, R⁷, R⁸, R⁹, R¹⁰, R¹¹, R¹², and R¹³ may independently —OR^{5A}, —OR^{6A}, —OR^{7A}, —OR^{8A}, —OR^{9A}, —OR^{10A}, —OR^{11A}, —OR^{12A}, and —OR^{13A} respectively. R^{5A}, R^{6A}, R^{7A}, R^{8A} R^{9A}, R^{10A}, R^{11A}, R^{12A}, and R^{13A} may independently be hydrogen (i.e. R⁵, R⁶, R⁷, R⁸, R⁹, R¹⁰, R¹¹, R¹², and R¹³ are independently —OH). R^{5A}, R^{6A}, R^{7A}, R^{8A}, R^{9A}, R^{10A}, R^{11A}, R^{12A}, and R^{13A} may independently be unsubstituted alkyl. R^{5A}, R^{6A}, R^{7A}, R^{8A}, R^{9A}, R^{1A}, R^{11A}, R^{12A}, and R^{13A} may independently be C₁-C₂₀ unsubstituted alkyl. R^{5A}, R^{6A}, R^{7A}, R^{8A}, R^{9A}, R^{10A}, R^{11A}, R^{12A}, and R^{13A} may independently be C₁-C₁₀ unsubstituted alkyl. R^{5A}, R^{6A}, R^{7A}, R^{8A}, R^{9A}, R^{10A}, R^{11A}, R^{12A}, and R^{13A} may independently be C₁-C₅ unsubstituted alkyl. R^{5A}, R^{6A}, R^{7A}, R^{8A}, R^{9A}, R^{1A}, R^{11A}, R^{12A}, and R^{13A} may independently be methyl. R^{5A}, R^{6A}, R^{7A}, R^{8A}, R^{9A}, R^{10A}, R^{11A}, R^{12A}, and R^{13A} may independently be an alcohol protecting group as described herein.

In embodiments, R⁵, R⁶, R⁷, R⁸, R⁹, R¹⁰, R¹¹, R¹², and R¹³ are independently halogen, —N₃, —NO₂, —CF₃, —CCl₃, —CBr₃, —CI₃, —CN, —OH, —NH₂, —COOH, —CONH₂, —SH, —SO₃H, —SO₄H, —SO₂NH₂, —NHNH₂, —ONH₂, —OCH₃, —NHC(O)NHNH₂. In embodiments, R⁵, R⁶, R⁷, R⁸, R⁹, R¹⁰, R¹¹, R¹², and R¹³ are independently halogen, —N₃, —NO₂, —CF₃, —CCl₃, —CBr₃, —CI₃, —CN, —OH, —NH₂, —COOH, —CONH₂, or —SH.

In embodiments, R⁴ is hydrogen. R⁴ may be C₁-C₁₀ unsubstituted alkyl. R⁴ may be C₁-C₅ unsubstituted alkyl. In embodiments, R⁴ is methyl. In embodiments, R⁴ is attached to a chiral carbon having an (S) stereochemistry. R⁴ may be (S) methyl.

X¹ may be —C(O). X¹ may be —C(R¹)(R²). X¹ may be —C(H)OH or —CH₂.

R¹ may be hydrogen or unsubstituted alkyl. R¹ may be hydrogen. R¹ may be unsubstituted alkyl. R¹ may be C₁-C₂₀ unsubstituted alkyl. R¹ may be C₁-C₁₀ unsubstituted alkyl. R¹ may be C₁-C₅ unsubstituted alkyl. In embodiments, R¹ is methyl. R¹ may be —OR^{1A}.

R² may be hydrogen or unsubstituted alkyl. R² may be hydrogen. R² may be unsubstituted alkyl. R² may be C₁-C₂₀ unsubstituted alkyl. R² may be C₁-C₁₀ unsubstituted alkyl. R² may be C₁-C₅ unsubstituted alkyl. In embodiments, R² is methyl. R² may be —OR^{2A}.

R^{1A} and R^{2A} may independently be hydrogen (i.e. R¹ and R² are independently —OH). R^{1A} and R^{2A} may independently be unsubstituted alkyl. R^{1A} and R^{2A} may independently be C₁-C₂₀ unsubstituted alkyl. R^{1A} and R^{2A} may independently be C₁-C₁₀ unsubstituted alkyl. R^{1A} and R^{2A} may independently be C₁-C₅ unsubstituted alkyl. R^{1A} and R^{2A} may independently be methyl. R^{1A} and R^{2A} may independently be an alcohol protecting group as described herein.

In embodiments, the compound of formula (I) has the formula:

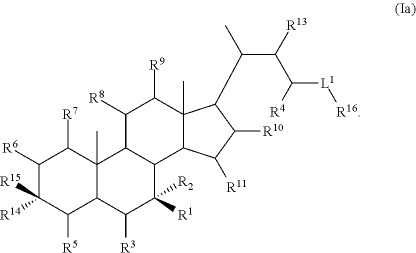

In embodiments, R¹ and R² are as defined by formula (Ia). When R¹ is —OR^{1A}, R² may be hydrogen. When R¹ is —OR^{1A}, R² may be —OR^{2A}. In embodiments, R¹ and R² are hydrogen. In embodiments, R¹ is not hydrogen. In embodiments, R² is not hydrogen. When R¹ is hydrogen, R² may be —OR^{2A}. When R¹ is —OR^{1A}, R² may be unsubstituted alkyl. When R¹ is —OR^{1A}, R² may be C₁-C₁₀ unsubstituted alkyl. When R¹ is —OR^{1A}, R² may be C₁-C₅ unsubstituted alkyl. When R¹ is —OR^{1A}, R² may be methyl. When R¹ is hydrogen, R² may be unsubstituted alkyl. When R¹ is hydrogen, R² may be C₁-C₁₀ unsubstituted alkyl. When R¹ is hydrogen, R² may be C₁-C₅ unsubstituted alkyl. When R¹ is hydrogen, R² is methyl. R^{1A} and R^{2A} are as described herein, including embodiments thereof. R^{1A} and R^{2A} may independently be hydrogen or C₁-C₅ unsubstituted alkyl. R^{1A} and R^{2A} may independently be an alcohol protecting group.

In embodiments, R¹ and R² are independently C₁-C₁₀ unsubstituted alkyl. In embodiments, when R¹ is C₁-C₁₀ unsubstituted alkyl, R² is C₁-C₅ unsubstituted alkyl. When R¹ is C₁-C₁₀ unsubstituted alkyl, R² may be methyl. When R¹ is C₁-C₁₀ unsubstituted alkyl, R² may be hydrogen. When R¹ is C₁-C₁₀ unsubstituted alkyl, R² may be —OR^{2A}. When R¹ is C₁-C₅ unsubstituted alkyl, R² may be C₁-C₁₀ unsubstituted alkyl. When R¹ is C₁-C₅ unsubstituted alkyl, R² may be C₁-C₅ unsubstituted alkyl. When R¹ is C₁-C₅ unsubstituted alkyl, R² may be methyl. When R¹ is C₁-C₅ unsubstituted alkyl, R² may be hydrogen. When R¹ is C₁-C₅ unsubstituted alkyl, R² may be —OR^{2A}. When R¹ is methyl, R² may be C₁-C₁₀ unsubstituted alkyl. When R¹ is methyl, R² may be C₁-C₅ unsubstituted alkyl. In embodiments, R¹ and R² are independently methyl. When R¹ is methyl, R² may be hydrogen. When R¹ is methyl, R² may be —OR^{2A}. R^{1A} and R^{2A} are as described herein, including embodiments thereof. R^{1A} and R^{2A} may independently be hydrogen or C₁-C₅ unsubstituted alkyl. R^{1A} and R^{2A} may independently be an alcohol protecting group.

When R² is —OR^{2A}, R¹ may be unsubstituted alkyl. When R² is —OR^{2A}, R¹ may be C₁-C₁₀ unsubstituted alkyl. When R² is —OR^{2A}, R¹ may be C₁-C₅ unsubstituted alkyl. When R² is —OR^{2A}, R¹ may be methyl. When R² is hydrogen, R¹ may be unsubstituted alkyl. When R² is hydrogen, R¹ may be C₁-C₁₀ unsubstituted alkyl. When R² is hydrogen, R¹ may be C₁-C₅ unsubstituted alkyl. When R² is hydrogen, R¹ may be methyl. R^{1A} and R^{2A} are as described herein, including embodiments thereof. R^{1A} and R^{2A} may independently be hydrogen or C₁-C₅ unsubstituted alkyl. R^{1A} and R^{2A} may independently be an alcohol protecting group.

When R² is C₁-C₁₀ unsubstituted alkyl, R¹ may be C₁-C₅ unsubstituted alkyl. When R² is C₁-C₁₀ unsubstituted alkyl, R¹ may be methyl. When R² is C₁-C₁₀ unsubstituted alkyl, R¹ may be hydrogen. When R² is C₁-C₁₀ unsubstituted alkyl, R¹ may be —OR^{1A}. When R² is C₁-C₅ unsubstituted alkyl, R¹ may be C₁-C₁₀ unsubstituted alkyl. In embodiments, R² and R¹ are independently C₁-C₅ unsubstituted alkyl. When R² is C₁-C₅ unsubstituted alkyl, R¹ may be methyl. When R² is C₁-C₅ unsubstituted alkyl, R¹ may be hydrogen. When R² is C₁-C₅ unsubstituted alkyl, R¹ may be —OR^{1A}. When R² is methyl, R¹ may be C₁-C₁₀ unsubstituted alkyl. When R² is methyl, R¹ may be C₁-C₅ unsubstituted alkyl. When R² is methyl, R¹ may be —OR^{1A}. R^{1A} and R^{2A} are as described herein, including embodiments thereof. R^{1A} and R^{2A} may independently be hydrogen or C₁-C₅ unsubstituted alkyl. R^{1A} and R^{2A} may independently be an alcohol protecting group.

In embodiments, R¹ and R² are independently halogen, —N₃, —NO₂, —CF₃, —CCl₃, —CBr₃, —CI₃, —CN, —OH, —NH₂, —COOH, —CONH₂, —SH, —SO₃H, —SO₄H, —SO₂NH₂, —NHNH₂, —ONH₂, —OCH₃, —NHC(O)NHNH₂. In embodiments, R¹ and R² are independently halogen, —N₃, —NO₂, —CF₃, —CCl₃, —CBr₃, —CI₃, —CN, —OH, —NH₂, —COOH, —CONH₂, or —SH.

X² may be —C(O). X² may be —C(R¹⁴)(R¹⁵). X² may be —C(H)OH or —CH₂.

In embodiments, R¹⁴ and R¹⁵ are as defined by formula (Ia). R¹⁴ may be hydrogen or unsubstituted alkyl. R¹⁴ may be hydrogen. R¹⁴ may be unsubstituted alkyl. R¹⁴ may be C₁-C₂₀ unsubstituted alkyl. R¹⁴ may be C₁-C₁₀ unsubstituted alkyl. R¹⁴ may be C₁-C₅ unsubstituted alkyl. In embodiments, R¹⁴ is methyl. R¹⁴ may be —OR^{14A}. R^{14A} may be hydrogen. R^{14A} may be unsubstituted alkyl (e.g. C₁-C₁₀ unsubstituted alkyl). R^{14A} may be an alcohol protecting group as described herein.

R¹⁵ may be hydrogen or unsubstituted alkyl. R⁵ may be hydrogen. R⁵ may be unsubstituted alkyl. R¹⁵ may be C₁-C₂₀ unsubstituted alkyl. R¹⁵ may be C₁-C₁₀ unsubstituted alkyl. R¹⁵ may be C₁-C₅ unsubstituted alkyl. In embodiments, R¹⁵ is methyl. R¹⁵ may be —OR^{15A}. R^{15A} may be hydrogen. R^{5A} may be unsubstituted alkyl (e.g. C₁-C₁₀ unsubstituted alkyl). R^{15A} may be an alcohol protecting group as described herein.

When R¹⁴ is —OR^{14A}, R¹⁵ may be hydrogen. When R¹⁴ is —OR^{14A}, R¹⁵ may be —OR^{5A}. In embodiments, R¹⁴ and R¹⁵ are hydrogen. When R¹⁴ is hydrogen, R¹⁵ may be —OR^{5A} When R¹⁴ is —OR^{14A}, R¹⁵ may be unsubstituted alkyl. When R¹⁴ is —OR^{14A}, R¹⁵ may be C₁-C₁₀ unsubstituted alkyl. When R¹⁴ is —OR^{14A}, R¹⁵ may be C₁-C₅ unsubstituted alkyl. When R¹⁴ is —OR^{14A}, R¹⁵ may be methyl. When R¹⁴ is hydrogen, R¹⁵ may be unsubstituted alkyl. When R¹⁴ is hydrogen, R¹⁵ may be C₁-C₁₀ unsubstituted alkyl. When R¹⁴ is hydrogen, R⁵ may C₁-C₅ unsubstituted alkyl. When R¹⁴ is hydrogen, R¹⁵ may be methyl. R^{14A} and R^{15A} are as described herein, including embodiments thereof. R^{14A} and R^{15A} may independently be hydrogen or C₁-C₅ unsubstituted alkyl. R^{14A} and R^{15A} may independently be an alcohol protecting group.

In embodiments, R¹⁴ and R¹⁵ are independently C₁-C₁₀ unsubstituted alkyl. When R¹⁴ is C₁-C₁₀ unsubstituted alkyl, R¹⁵ may C₁-C₅ unsubstituted alkyl. When R¹⁴ is C₁-C₁₀ unsubstituted alkyl, R¹⁵ may methyl. When R¹⁴ is C₁-C₁₀ unsubstituted alkyl, R¹⁵ may be hydrogen. When R¹⁴ is C₁-C₁₀ unsubstituted alkyl, R¹⁵ may be —OR^{15A}. When R¹⁴ is C₁-C₅ unsubstituted alkyl, R¹⁵ may be C₁-C₁₀ unsubstituted alkyl. In embodiments, R¹⁴ and R¹⁵ are independently C₁-C₅ unsubstituted alkyl. When R¹⁴ is C₁-C₅ unsubstituted alkyl, R¹⁵ may be methyl. When R¹⁴ is C₁-C₅ unsubstituted alkyl, R¹⁵ may be hydrogen. When R¹⁴ is C₁-C₅ unsubstituted alkyl, R¹⁵ may be —OR^{15A}. When R¹⁴ is methyl, R¹⁵ may be C₁-C₁₀ unsubstituted alkyl. When R¹⁴ is methyl, R¹⁵ may be C₁-C₅ unsubstituted alkyl. In embodiments, R¹⁴ and R¹⁵ are methyl. When R¹⁴ is methyl, R¹⁵ may be hydrogen. When R¹⁴ is methyl, R¹⁵ may be —OR^{15A}.

When R¹⁵ is —OR^{5A}, R¹⁴ may be unsubstituted alkyl. When R¹⁵ is —OR^{5A}, R¹⁴ may be C₁-C₁₀ unsubstituted alkyl. When R¹⁵ is —OR^{15A}, R¹⁴ is C₁-C₅ unsubstituted alkyl. When R¹⁵ is —OR^{15A}, R¹⁴ may be methyl. When R¹⁵ is hydrogen, R¹⁴ may be unsubstituted alkyl. When R¹⁵ is hydrogen, R¹⁴ may be C₁-C₁₀ unsubstituted alkyl. When R¹⁵ is hydrogen, R¹⁴ may be C₁-C₅ unsubstituted alkyl. When R¹⁵ is hydrogen, R¹⁴ may be methyl.

When R¹⁵ is C₁-C₁₀ unsubstituted alkyl, R¹⁴ may be C₁-C₅ unsubstituted alkyl. When R¹⁵ is C₁-C₁₀ unsubstituted alkyl, R¹⁴ may be methyl. When R¹⁵ is C₁-C₁₀ unsubstituted alkyl, R¹⁴ may be hydrogen. When R¹⁵ is C₁-C₁₀ unsubstituted alkyl, R¹⁴ may be —OR^{14A}. When R¹⁵ is C₁-C₅ unsubstituted alkyl, R¹⁴ may be C₁-C₁₀ unsubstituted alkyl. When R¹⁵ is C₁-C₅ unsubstituted alkyl, R¹⁴ may be methyl. When R¹⁵ is C₁-C₅ unsubstituted alkyl, R¹⁴ may be hydrogen. When R¹⁵ is C₁-C₅ unsubstituted alkyl, R¹⁴ may be —OR^{14A}. When R¹⁵ is methyl, R¹⁴ may be C₁-C₁₀ unsubstituted alkyl. When R¹⁵ is methyl, R¹⁴ may be C₁-C₅ unsubstituted alkyl. In embodiments, R¹⁵ and R¹⁴ are independently methyl. When R¹⁵ is methyl, R¹⁴ may be hydrogen. When R¹⁵ is methyl, R¹⁴ may be —OR^{14A}.

In embodiments, R¹⁴ and R¹⁵ are independently halogen, —N₃, —NO₂, —CF₃, —CCl₃, —CBr₃, —CI₃, —CN, —OH, —NH₂, —COOH, —CONH₂, —SH, —SO₃H, —SO₄H, —SO₂NH₂, —NHNH₂, —ONH₂, —OCH₃, —NHC(O)NHNH₂. In embodiments, R¹⁴ and R¹⁵ are independently halogen, —N₃, —NO₂, —CF₃, —CCl₃, —CBr₃, —CI₃, —CN, —OH, —NH₂, —COOH, —CONH₂, or —SH.

In embodiments, R^{1A}, R^{2A}, R^{3A}, R^{5A}, R^{6A}, R^{7A}, R^{8A}, R^{9A}, R^{10A}, R^{11A}, R^{12A}, R^{13A}, R^{14A} R^{15A}, and R^{16A} are hydrogen. R^{1A}, R^{2A}, R^{3A}, R^{5A}, R^{6A}, R^{7A}, R^{8A}, R^{9A}, R^{10A}, R^{11A}, R^{12A}, R^{13A} R^{14A}, R^{15A}, and R^{16A} may independently be an alcohol protecting group.

In embodiments, R¹ and R² are hydrogen and R¹⁴ is —OR^{14A}. R^{14A} may be hydrogen or an alcohol protecting group as described herein. R^{14A} may be hydrogen. R^{14A} may be unsubstituted alkyl. In embodiments, R¹ is —OR^{1A} and R¹⁴ is —OR^{14A}. R^{1A} is as described herein, including embodiments thereof. R^{14A} is as described herein, including embodiments thereof. In embodiments, R^{1A} and R^{14A} are independently hydrogen or an alcohol protecting group as described herein. R^{1A} and R^{14A} may independently be substituted or unsubstituted alkyl (e.g. C₁-C₅ substituted or unsubstituted alkyl).

R³ may be hydrogen. R³ may be unsubstituted alkyl. R³ may be C₁-C₁₀ unsubstituted alkyl. R³ is C₁-C₅ unsubstituted alkyl. R³ may be ethyl. R³ may be attached to a chiral carbon having α-stereochemistry. R³ may be —OR^{3A}. R^{3A} may be hydrogen. R^{3A} may be unsubstituted alkyl. R^{3A} may be C₁-C₁₀ unsubstituted alkyl. R^{3A} may be C₁-C₅ unsubstituted alkyl. R^{3A} may be an alcohol protecting group as described herein, including embodiments thereof.

In embodiments, R³ and R⁴ are independently C₁-C₁₀ unsubstituted alkyl. When R³ is C₁-C₁₀ unsubstituted alkyl, R⁴ may be C₁-C₅ unsubstituted alkyl. When R³ is C₁-C₁₀ unsubstituted alkyl, R⁴ may be methyl. When R³ is C₁-C₁₀ unsubstituted alkyl, R⁴ may be hydrogen. When R³ is C₁-C₅ unsubstituted alkyl, R⁴ may be C₁-C₁₀ unsubstituted alkyl. In embodiments, R³ and R⁴ are independently C₁-C₅ unsubstituted alkyl. When R³ is C₁-C₅ unsubstituted alkyl, R⁴ may be methyl. When R³ is C₁-C₅ unsubstituted alkyl, R⁴ may be hydrogen. When R³ is ethyl, R⁴ may be C₁-C₁₀ unsubstituted alkyl. When R³ is ethyl, R⁴ may be C₁-C₅ unsubstituted alkyl. When R³ is ethyl, R⁴ may be methyl. When R³ is ethyl, R⁴ may be hydrogen. R⁴ may be attached to a chiral carbon having an a stereochemistry (e.g. α-ethyl).

When R⁴ is C₁-C₁₀ unsubstituted alkyl, R³ may be C₁-C₅ unsubstituted alkyl. When R⁴ is C₁-C₁₀ unsubstituted alkyl, R³ may be ethyl. When R⁴ is C1-C10 unsubstituted alkyl, R³ may be methyl. When R⁴ is C₁-C₁₀ unsubstituted alkyl, R³ may be hydrogen. When R⁴ is C₁-C₅ unsubstituted alkyl, R³ may be C₁-C₁₀ unsubstituted alkyl. When R⁴ is C₁-C₅ unsubstituted alkyl, R³ may be ethyl. When R⁴ is C₁-C₅ unsubstituted alkyl, R³ may be methyl. When R⁴ is C₁-C₅ unsubstituted alkyl, R³ may be hydrogen. When R⁴ is methyl, R³ may be C₁-C₁₀ unsubstituted alkyl. When R⁴ is methyl, R³ may be C₁-C₅ unsubstituted alkyl. When R⁴ is methyl, R³ may be ethyl. In embodiments, R⁴ and R³ are methyl. When R⁴ is methyl, R³ may be hydrogen.

In embodiments, R³ and R⁴ are independently halogen, —N₃, —NO₂, —CF₃, —CCl₃, —CBr₃, —CI₃, —CN, —OH, —NH₂, —COOH, —CONH₂, —SH, —SO₃H, —SO₄H, —SO₂NH₂, —NHNH₂, —ONH₂, —OCH₃, —NHC(O)NHNH₂. In embodiments, R³ and R⁴ are independently halogen, —N₃, —NO₂, —CF₃, —CCl₃, —CBr₃, —CI₃, —CN, —OH, —NH₂, —COOH, —CONH₂, or —SH.

X¹ and X² may independently be —C(O). In embodiments, X¹ and X² are —C(O). In embodiments, X¹ is —C(O) and X² is —C(O) or —C(R¹⁴)(R¹⁵). In embodiments, X¹ and X² are —C(O). When X¹ is —C(O), X² may be —C(O) or —C(R¹⁴)(R⁵). R¹⁴ and R¹⁵ are as described herein, including embodiments thereof. Thus, in embodiments, when X¹ is —C(O), X² may be —C(H)OH or —CH₂. When X² is —C(O), X¹ may be —C(R¹)(R²). R¹ and R² are as described herein, including embodiments thereof. Thus, in embodiments, when X² is —C(O), X¹ may be C(H)OH, or —CH₂. In embodiments, X¹ is —C(R¹)(R²) and X² is —C(R¹⁴)(R¹⁵). R¹, R², R¹⁴, and R¹⁵ are as described herein, including embodiments thereof. Thus, in embodiments, X¹ is —C(H)OH or —CH₂ and X² is —C(H)OH or —CH₂. In embodiments, X¹ and X² are —C(H)OH. When X² is —C(H)OH, X¹ may be —CH₂.

L¹ may be —C(O)—, —C(O)O—, or —C(O)NH—. L¹ may be —C(O)—. L¹ may be —C(O)O—. L¹ may be —C(O)NH—. L¹ may be —CH₂—. R¹⁶ may be hydrogen, —OR^{16A}, —NHR^{16A}, an alcohol protecting group, or a carboxylate protecting group. R¹⁶ may be hydrogen. R¹⁶ may be —OR^{16A}, where R^{16A} is as described herein, including embodiments thereof. R¹⁶ may be —NHR^{16A}, where R^{16A} is as described herein, including embodiments thereof. In embodiments, R¹⁶ is a hydrogen or a carboxylate protecting group, and L¹ is —C(O)O—. When L¹ is —C(O)— or —C(O)NH—, R¹⁶ may be substituted or unsubstituted alkyl or substituted or unsubstituted heteroalkyl. R¹⁶ is as described herein, including embodiments thereof. In embodiments, L¹ is —C(O)O—; and R¹⁶ is hydrogen, or a carboxylate protecting group.

R¹⁶ may be substituted or unsubstituted alkyl. R¹⁶ may be unsubstituted alkyl. R¹⁶ may be R¹⁷-substituted or unsubstituted alkyl. R¹⁶ may be substituted or unsubstituted alkyl. R¹⁶ may be substituted or unsubstituted C₁-C₂₀ alkyl. R¹⁶ may be R¹⁷-substituted or unsubstituted C₁-C₂₀ alkyl. R¹⁶ may be substituted or unsubstituted C₁-C₁₀ alkyl. R¹⁶ may be R¹⁷-substituted or unsubstituted C₁-C₁₀ alkyl. R¹⁶ may be substituted or unsubstituted C₁-C₅ alkyl. R¹⁶ may be R¹⁷-substituted or unsubstituted C₁-C₅ alkyl.

R¹⁶ may be substituted or unsubstituted heteroalkyl. R¹⁶ may be unsubstituted heteroalkyl. R¹⁶ may be substituted or unsubstituted 2 to 20 membered heteroalkyl. R¹⁶ may be R¹⁷-substituted or unsubstituted 2 to 20 membered heteroalkyl. R¹⁶ may be substituted or unsubstituted 2 to 10 membered heteroalkyl. R¹⁶ may be R¹⁷-substituted or unsubstituted 2 to 10 membered heteroalkyl. R¹⁶ may be substituted or unsubstituted 2 to 6 membered heteroalkyl. R¹⁶ may be R¹⁷-substituted or unsubstituted 2 to 6 membered heteroalkyl.

R¹⁶ may be substituted or unsubstituted cycloalkyl. R¹⁶ may be unsubstituted cycloalkyl. R¹⁶ may be R¹⁷-substituted or unsubstituted 3 to 20 membered cycloalkyl. R¹⁶ may be substituted or unsubstituted 3 to 20 membered cycloalkyl. R¹⁶ may be substituted or unsubstituted 3 to 10 membered cycloalkyl. R¹⁶ may be R¹⁷-substituted or unsubstituted 3 to 10 membered cycloalkyl. R¹⁶ may be substituted or unsubstituted 3 to 6 membered cycloalkyl. R¹⁶ may be R¹⁷-substituted or unsubstituted 3 to 6 membered cycloalkyl.

R¹⁶ may be substituted or unsubstituted heterocycloalkyl. R¹⁶ may be unsubstituted heterocycloalkyl. R¹⁶ may be substituted or unsubstituted 3 to 20 membered heterocycloalkyl. R¹⁶ may be R¹⁷-substituted or unsubstituted 3 to 20 membered heterocycloalkyl. R¹⁶ may be substituted or unsubstituted 3 to 10 membered heterocycloalkyl. R¹⁶ may be R⁷-substituted or unsubstituted 3 to 10 membered heterocycloalkyl. R¹⁶ may be substituted or unsubstituted 3 to 6 membered heterocycloalkyl. R¹⁶ may be R¹⁷-substituted or unsubstituted 3 to 6 membered heterocycloalkyl.

R¹⁶ may be substituted or unsubstituted aryl. R¹⁶ may be unsubstituted aryl. R¹⁶ may be substituted or unsubstituted 5 to 10 membered aryl. R¹⁶ may be R¹⁷-substituted or unsubstituted 5 to 10 membered aryl. R¹⁶ may be substituted or unsubstituted 5 to 8 membered aryl. R¹⁶ may be R¹⁷-substituted or unsubstituted 5 to 8 membered aryl. R¹⁶ may be substituted or unsubstituted 5 or 6 membered aryl. R¹⁶ may be R¹⁷-substituted or unsubstituted 5 or 6 membered aryl.

R¹⁶ may be substituted or unsubstituted heteroaryl. R¹⁶ may be unsubstituted heteroaryl. R¹⁶ may be substituted or unsubstituted 5 to 10 membered heteroaryl. R¹⁶ may be R¹⁷-substituted or unsubstituted 5 to 10 membered heteroaryl. R¹⁶ may be substituted or unsubstituted 5 to 8 membered heteroaryl. R¹⁶ may be R¹⁷-substituted or unsubstituted 5 to 8 membered heteroaryl. R¹⁶ may be substituted or unsubstituted 5 or 6 membered heteroaryl. R¹⁶ may be R¹⁷-substituted or unsubstituted 5 or 6 membered heteroaryl.

R¹⁷ is hydrogen, halogen, —N₃, —CF₃, —CCl₃, —CBr₃, —CI₃, —CN, —CHO, —OH, —NH₂, —COOH, —CONH₂, —NO₂, —SH, —SO₂Cl, —SO₃H, —SO₄H, —SO₂NH₂, —NHNH₂, —ONH₂, —NHC(O)NHNH₂, R¹⁸-substituted or unsubstituted alkyl (e.g. C₁-C₈ alkyl), R¹⁸-substituted or unsubstituted heteroalkyl (e.g. 2 to 8 membered heteroalkyl), R¹⁸-substituted or unsubstituted cycloalkyl (e.g. 3 to 8 membered cycloalkyl), R¹⁸-substituted or unsubstituted heterocycloalkyl (e.g. 3 to 8 membered heterocycloalkyl), R¹⁸-substituted or unsubstituted aryl (e.g. 5 or 6 membered aryl), or R¹⁸-substituted or unsubstituted heteroaryl (e.g. 5 or 6 membered heteroaryl).

R¹⁸ is hydrogen, halogen, —N₃, —CF₃, —CCl₃, —CBr₃, —CI₃, —CN, —CHO, —OH, —NH₂, —COOH, —CONH₂, —NO₂, —SH, —SO₂Cl, —SO₃H, —SO₄H, —SO₂NH₂, —NHNH₂, —ONH₂, —NHC(O)NHNH₂, R¹⁹-substituted or unsubstituted alkyl (e.g. C₁-C₈ alkyl), R¹⁹-substituted or unsubstituted heteroalkyl (e.g. 2 to 8 membered heteroalkyl), R¹⁹-substituted or unsubstituted cycloalkyl (e.g. 3 to 8 membered cycloalkyl), R¹⁹-substituted or unsubstituted heterocycloalkyl (e.g. 3 to 8 membered heterocycloalkyl), R¹⁹-substituted or unsubstituted aryl (e.g. 5 or 6 membered aryl), or R¹⁹-substituted or unsubstituted heteroaryl (e.g. 5 or 6 membered heteroaryl).

R¹⁹ is hydrogen, halogen, —N₃, —CF₃, —CCl₃, —CBr₃, —CI₃, —CN, —CHO, —OH, —NH₂, —COOH, —CONH₂, —NO₂, —SH, —SO₂Cl, —SO₃H, —SO₄H, —SO₂NH₂, —NHNH₂, —ONH₂, —NHC(O)NHNH₂, R²⁰-substituted or unsubstituted alkyl (e.g. C₁-C₈ alkyl), R²⁰-substituted or unsubstituted heteroalkyl (e.g. 2 to 8 membered heteroalkyl), R²⁰-substituted or unsubstituted cycloalkyl (e.g. 3 to 8 membered cycloalkyl), R²⁰-substituted or unsubstituted heterocycloalkyl (e.g. 3 to 8 membered heterocycloalkyl), R²⁰-substituted or unsubstituted aryl (e.g. 5 or 6 membered aryl), or R²⁰-substituted or unsubstituted heteroaryl (e.g. 5 or 6 membered heteroaryl).

R²⁰ is hydrogen, halogen, —N₃, —CF₃, —CCl₃, —CBr₃, —CI₃, —CN, —CHO, —OH, —NH₂, —COOH, —CONH₂, —NO₂, —SH, —SO₂Cl, —SO₃H, —SO₄H, —SO₂NH₂, —NHNH₂, —ONH₂, —NHC(O)NHNH₂, unsubstituted alkyl (e.g. C₁-C₈ alkyl), unsubstituted heteroalkyl (e.g. 2 to 8 membered heteroalkyl), unsubstituted cycloalkyl (e.g. 3 to 8 membered cycloalkyl), unsubstituted heterocycloalkyl (e.g. 3 to 8 membered heterocycloalkyl), unsubstituted aryl (e.g. 5 or 6 membered aryl), or unsubstituted heteroaryl (e.g. 5 or 6 membered heteroaryl).

In embodiments, R¹⁶ is R¹⁷-substituted alkyl. R¹⁷ may halogen, —N₃, —CF₃, —CCl₃, —CBr₃, —CI₃, —CN, —CHO, —OH, —NH₂, —COOH, —CONH₂, —NO₂, —SH, —SO₂Cl, —SO₃H, —SO₄H, —SO₂NH₂, —NHNH₂, —ONH₂, —NHC(O)NHNH₂, R¹⁸-substituted alkyl, R¹⁸-substituted heteroalkyl, R¹⁸-substituted cycloalkyl, R¹⁸-substituted heterocycloalkyl, or R¹⁸-substituted aryl. In embodiments, R¹⁶ is a taurine moiety (e.g. —NH(CH₂)₂SO₃H) or a glycine moiety (e.g. —NHCH₂COOH) or a pharmaceutically acceptable salt thereof. In embodiments, R¹⁶ is a taurine moiety (e.g. —NH(CH₂)₂SO₃H). In embodiments, R¹⁶ is a glycine moiety (e.g. —NHCH₂COOH).

R^{16A} may be hydrogen, halogen, —N₃, —CF₃, —CCl₃, —CBr₃, —CI₃, —CN, —CHO, —OH, —NH₂, —COOH, —CONH₂, —NO₂, —SH, —SO₂Cl, —SO₃H, —SO₄H, —SO₂NH₂, —NHNH₂, —ONH₂, —NHC(O)NHNH₂. R^{16A} may be an alcohol protecting group, or a carboxylate protecting group. R^{16A} may be substituted or unsubstituted alkyl (e.g. C₁-C₈ alkyl), substituted or unsubstituted heteroalkyl (e.g. 2 to 8 membered heteroalkyl), substituted or unsubstituted cycloalkyl, substituted or unsubstituted heterocycloalkyl, substituted or unsubstituted aryl, or substituted or unsubstituted heteroaryl.

In embodiments, R^{16A} is substituted or unsubstituted alkyl. R^{16A} may be substituted or unsubstituted alkyl. R^{16A} may be R²¹— substituted or unsubstituted alkyl. R^{16A} may be substituted or unsubstituted C₁-C₁₀ alkyl. R^{16A} may be R²¹-substituted or unsubstituted C₁-C₁₀ alkyl. R^{16A} may be substituted or unsubstituted C₁-C₅ alkyl. R^{16A} may be R²¹-substituted or unsubstituted C₁-C₅ alkyl.

In embodiments, R^{16A} is substituted or unsubstituted heteroalkyl. R^{16A} may be R²¹-substituted or unsubstituted heteroalkyl. R^{16A} may be substituted or unsubstituted 2 to 10 membered heteroalkyl. R^{16A} may be R²¹-substituted or unsubstituted 2 to 10 membered heteroalkyl. R^{16A} may be substituted or unsubstituted 2 to 6 membered heteroalkyl. R^{16A} may be R²¹-substituted or unsubstituted 2 to 6 membered heteroalkyl.

In embodiments, R^{16A} is substituted or unsubstituted cycloalkyl. R^{16A} may be R²¹-substituted or unsubstituted cycloalkyl. R^{16A} may be substituted or unsubstituted 3 to 10 membered cycloalkyl. R^{16A} may be R²¹-substituted or unsubstituted 3 to 10 membered cycloalkyl. R^{16A} may be substituted or unsubstituted 3 to 6 membered cycloalkyl. R^{16A} may be R²¹-substituted or unsubstituted 3 to 6 membered cycloalkyl.

In embodiments, R^{16A} is substituted or unsubstituted heterocycloalkyl. R^{16A} may be R²¹-substituted or unsubstituted heterocycloalkyl. R^{16A} may be substituted or unsubstituted 3 to 10 membered heterocycloalkyl. R^{16A} may be R²¹-substituted or unsubstituted 3 to 10 membered heterocycloalkyl. R^{16A} may be substituted or unsubstituted 3 to 6 membered heterocycloalkyl. R^{16A} may be R²¹-substituted or unsubstituted 3 to 6 membered heterocycloalkyl.

In embodiments, R^{16A} is substituted or unsubstituted aryl. R^{16A} may be R²¹-substituted or unsubstituted aryl. R^{16A} may be substituted or unsubstituted 5 to 10 membered aryl. R^{16A} may be R²¹-substituted or unsubstituted 5 to 10 membered aryl. R^{16A} may be substituted or unsubstituted 5 or 6 membered aryl. R^{16A} may be R²¹-substituted or unsubstituted 5 or 6 membered aryl.

In embodiments, R^{16A} is substituted or unsubstituted heteroaryl. R^{16A} may be R²¹-substituted or unsubstituted heteroaryl. R^{16A} may be substituted or unsubstituted 5 to 10 membered heteroaryl. R^{16A} may be R²¹-substituted or unsubstituted 5 to 10 membered heteroaryl. R^{16A} may be substituted or unsubstituted 5 or 6 membered heteroaryl. R^{16A} may be R²¹-substituted or unsubstituted 5 or 6 membered heteroaryl.

R²¹ is hydrogen, halogen, —N₃, —CF₃, —CCl₃, —CBr₃, —CI₃, —CN, —CHO, —OH, —NH₂, —COOH, —CONH₂, —NO₂, —SH, —SO₂Cl, —SO₃H, —SO₄H, —SO₂NH₂, —NHNH₂, —ONH₂, —NHC(O)NHNH₂, R²²-substituted or unsubstituted alkyl (e.g. C₁-C₈ alkyl), R²²-substituted or unsubstituted heteroalkyl (e.g. 2 to 8 membered heteroalkyl), R²²-substituted or unsubstituted cycloalkyl (e.g. 3 to 8 membered cycloalkyl), R²²-substituted or unsubstituted heterocycloalkyl (e.g. 3 to 8 membered heterocycloalkyl), R²²-substituted or unsubstituted aryl (e.g. 5 or 6 membered aryl), or R²²-substituted or unsubstituted heteroaryl (e.g. 5 or 6 membered heteroaryl).

R²² is hydrogen, halogen, —N₃, —CF₃, —CCl₃, —CBr₃, —CI₃, —CN, —CHO, —OH, —NH₂, —COOH, —CONH₂, —NO₂, —SH, —SO₂Cl, —SO₃H, —SO₄H, —SO₂NH₂, —NHNH₂, —ONH₂, —NHC(O)NHNH₂, R²³-substituted or unsubstituted alkyl (e.g. C₁-C₈ alkyl), R²³-substituted or unsubstituted heteroalkyl (e.g. 2 to 8 membered heteroalkyl), R²³-substituted or unsubstituted cycloalkyl (e.g. 3 to 8 membered cycloalkyl), R²³-substituted or unsubstituted heterocycloalkyl (e.g. 3 to 8 membered heterocycloalkyl), R²³-substituted or unsubstituted aryl (e.g. 5 or 6 membered aryl), or R²³-substituted or unsubstituted heteroaryl (e.g. 5 or 6 membered heteroaryl).

R²³ is hydrogen, halogen, —N₃, —CF₃, —CCl₃, —CBr₃, —CI₃, —CN, —CHO, —OH, —NH₂, —COOH, —CONH₂, —NO₂, —SH, —SO₂Cl, —SO₃H, —SO₄H, —SO₂NH₂, —NHNH₂, —ONH₂, —NHC(O)NHNH₂, R²⁴-substituted or unsubstituted alkyl (e.g. C₁-C₈ alkyl), R²⁴-substituted or unsubstituted heteroalkyl (e.g. 2 to 8 membered heteroalkyl), R²⁴-substituted or unsubstituted cycloalkyl (e.g. 3 to 8 membered cycloalkyl), R²⁴-substituted or unsubstituted heterocycloalkyl (e.g. 3 to 8 membered heterocycloalkyl), R²⁴-substituted or unsubstituted aryl (e.g. 5 or 6 membered aryl), or R²⁴-substituted or unsubstituted heteroaryl (e.g. 5 or 6 membered heteroaryl).

R²⁴ is hydrogen, halogen, —N₃, —CF₃, —CCl₃, —CBr₃, —CI₃, —CN, —CHO, —OH, —NH₂, —COOH, —CONH₂, —NO₂, —SH, —SO₂Cl, —SO₃H, —SO₄H, —SO₂NH₂, —NHNH₂, —ONH₂, —NHC(O)NHNH₂, unsubstituted alkyl (e.g. C₁-C₈ alkyl), unsubstituted heteroalkyl (e.g. 2 to 8 membered heteroalkyl), unsubstituted cycloalkyl (e.g. 3 to 8 membered cycloalkyl), unsubstituted heterocycloalkyl (e.g. 3 to 8 membered heterocycloalkyl), unsubstituted aryl (e.g. 5 or 6 membered aryl), or unsubstituted heteroaryl (e.g. 5 or 6 membered heteroaryl).

In embodiments, R^{16A} is R²¹-substituted alkyl. R²¹ may halogen, —N₃, —CF₃, —CCl₃, —CBr₃, —CI₃, —CN, —CHO, —OH, —NH₂, —COOH, —CONH₂, —NO₂, —SH, —SO₂Cl, —SO₃H, —SO₄H, —SO₂NH₂, —NHNH₂, —ONH₂, —NHC(O)NHNH₂, R²²-substituted alkyl, R²²-substituted heteroalkyl, R²²-substituted cycloalkyl, R²²-substituted heterocycloalkyl, or R²² substituted aryl.

In embodiments the compound of formula (I) has the formula:

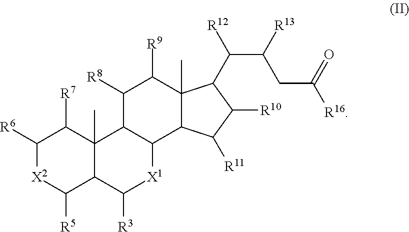

R¹⁶ is a carboxylate protecting group. In embodiments, at least one of R¹, R², R³, R⁵, R⁶, R⁷, R⁸, R⁹, R¹⁰, R¹¹, R¹², R¹³, R¹⁴, or R¹⁵ is respectively —OR^{1A}, —OR^{2A}, —OR^{3A}, —OR^{5A}, OR^{6A}, —OR^{7A}, —OR^{8A}, —OR^{9A}, —OR^{10A}, —OR^{11A}, —OR^{12A}, —OR^{13A}, —OR^{14A}, or —OR^{15A}. R^{1A}, R^{2A}, R^{3A}, R^{5A} R^{6A}, R^{7A}, R^{8A}, R^{9A}, R^{10A}, R^{11A}, R^{12A}, R^{13A}, R^{14A}, or R^{15A} may independently be hydrogen or an alcohol protecting group as described herein, including embodiments thereof. R^{1A}, R^{2A}, R^{3A}, R^{5A}, R^{6A}, R^{7A}, R^{8A}, R^{9A}, R^{10A}, R^{11A}, R^{12A}, R^{13A}, R^{14A}, or R^{15A} may independently be hydrogen. R¹R^{1A}, R^{2A}, R^{3A}, R^{5A}, R^{6A}, R^{7A}, R^{8A}, R^{9A}, R^{1A}, R^{11A}, R^{12A}, R^{13A}, R^{14A}, or R^{15A} may independently be an alcohol protecting group as described herein, including embodiments thereof. In embodiments, R^{1A}, R^{2A}, R^{3A}, R^{5A}, R^{6A}, R^{7A}, R^{8A}, R^{9A}, R^{10A}, R^{11A}, R^{12A}, R^{13A}, R^{14A}, or R^{15A} are independently unsubstituted C₁-C₅ alkyl.

In embodiments, the compound of formula (I) has the formula:

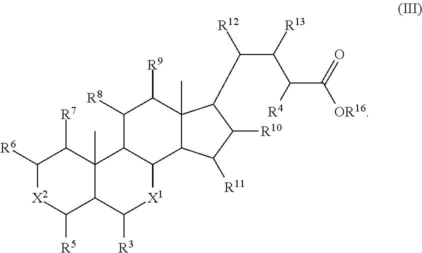

or a pharmaceutically acceptable salt thereof.

In the compound of formula (III), R⁴ may be methyl. R³ may be ethyl. X¹ may be —C(R′)(R²). When X¹ is —C(R¹)(R²), and R¹ is —OH, X² may be —C(R¹⁴)(R⁵), where R¹⁴ is —OH. X¹ may be —C(O). When X¹ is —C(O), X² may be —C(R¹⁴)(R¹⁵) wherein R¹⁴ is —OH. In embodiments, X¹ and X² are —C(O). X¹ may be —CH₂. When X¹ is —CH₂, X² may be —C(R¹⁴)(R¹⁵). R¹⁴ may be —OH.

The compound of formula (III) may have the formula:

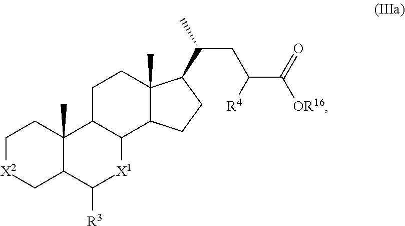

The compound of formula (IIIa) may have formula:

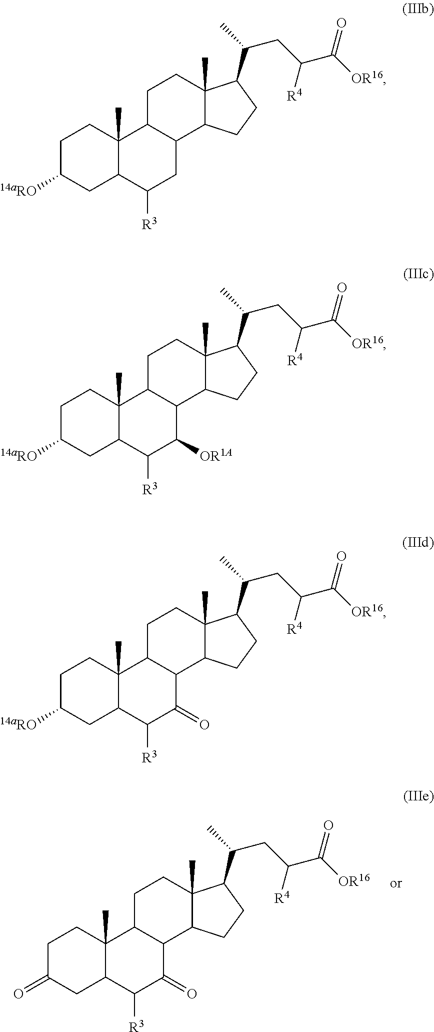

a pharmaceutically acceptable salt thereof.

In embodiments R¹⁶ is hydrogen or a carboxylate protecting group. R^{1A} and R^{14A} may independently be hydrogen or an alcohol deprotecting group. In embodiments, R⁴ is methyl. R⁴ may be attached to a chiral carbon having an (S) stereochemistry (e.g. (S)-methyl). In embodiments R³ is ethyl.

The compound of formula (I) may have formula:

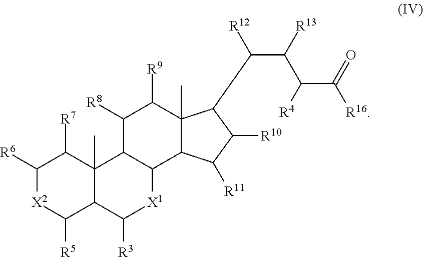

or a pharmaceutically acceptable salt thereof.

In the compound of formula (IV), R⁴ may be methyl. R³ may be ethyl. X¹ may be —C(R¹)(R²). When X¹ is —C(R¹)(R²), and R¹ is —OH, X² may be —C(R¹⁴)(R¹⁵), where R¹⁴ is —OH. X¹ may be —C(O). When X¹ is —C(O), X² may be —C(R¹⁴)(R¹⁵). R¹⁴ may be —OH. In embodiments, X¹ and X² are —C(O). X¹ may be —CH₂. When X¹ is —CH₂, X² may be —C(R¹⁴)(R¹⁵). R¹⁴ may be —OH.

The compound of formula (IV) may have formula:

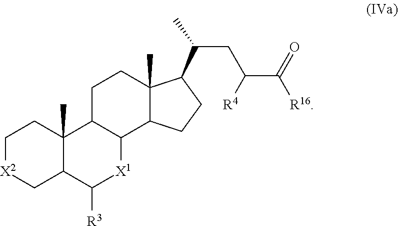

The compound of formula (IV) may have formula:

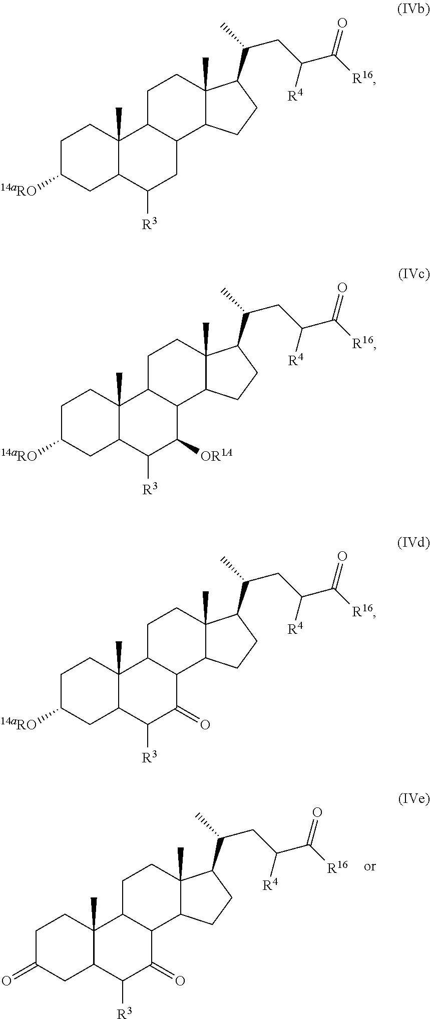

a pharmaceutically acceptable salt thereof.

In embodiments R¹⁶ is substituted or unsubstituted alkyl, substituted or unsubstituted heteroalkyl, —NHR^{16A} or —OR^{16A}. R¹⁶ may be substituted or unsubstituted alkyl. R¹⁶ may be substituted or unsubstituted heteroalkyl. In embodiments, R¹⁶ is a taurine moiety or a glycine moiety. R^{16A} may be hydrogen, substituted or unsubstituted alkyl or substituted or unsubstituted heteroalkyl. R^{16A} may a taurine moiety (e.g. —NH(CH₂)₂SO₃H) or a glycine moiety (e.g. —NHCH₂COOH) or a pharmaceutically acceptable salt thereof. R^{1A} and R^{14A} may independently be hydrogen or an alcohol deprotecting group. In embodiments, R⁴ is methyl. R⁴ may be attached to a chiral carbon having an (S) stereochemistry (e.g. (S)-methyl). In embodiments R³ is ethyl. R³ may be attached to a chiral carbon having an a stereochemistry (e.g. α-ethyl).

The compound of formula (I) may have formula:

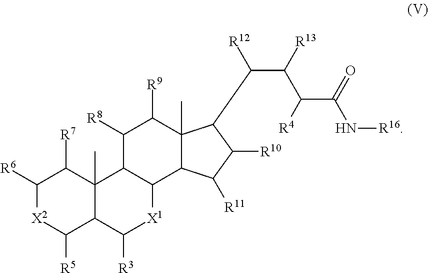

or a pharmaceutically acceptable salt thereof.

In embodiments, the compound of formula (I) has formula:

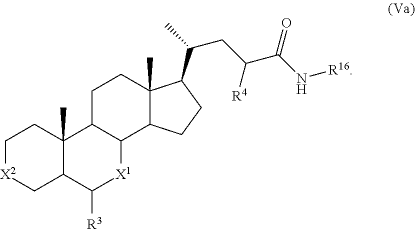

X¹, X², R³, R⁴, and R⁵ are as described herein, including embodiments thereof.

In embodiments, the compound of formula (V) has formula:

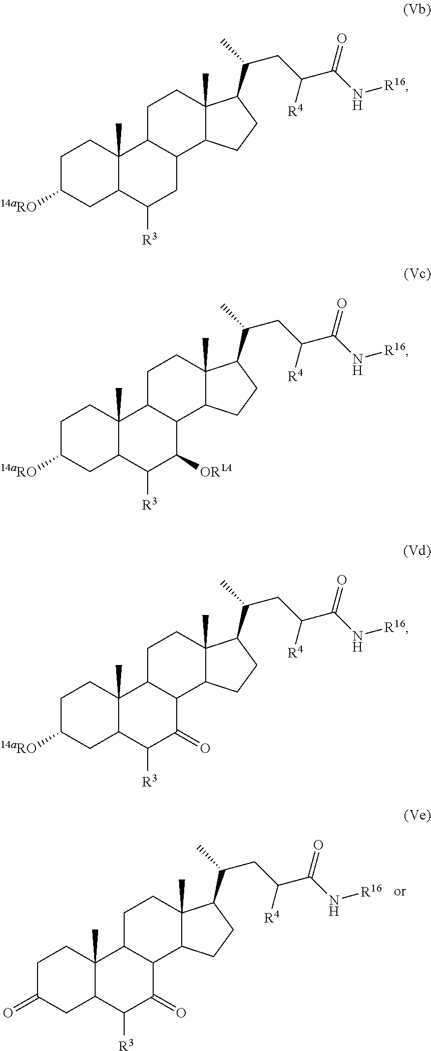

In embodiments, the compounds herein (e.g. of formula (I), (II), (III), and (IV), including embodiments thereof) has formula:

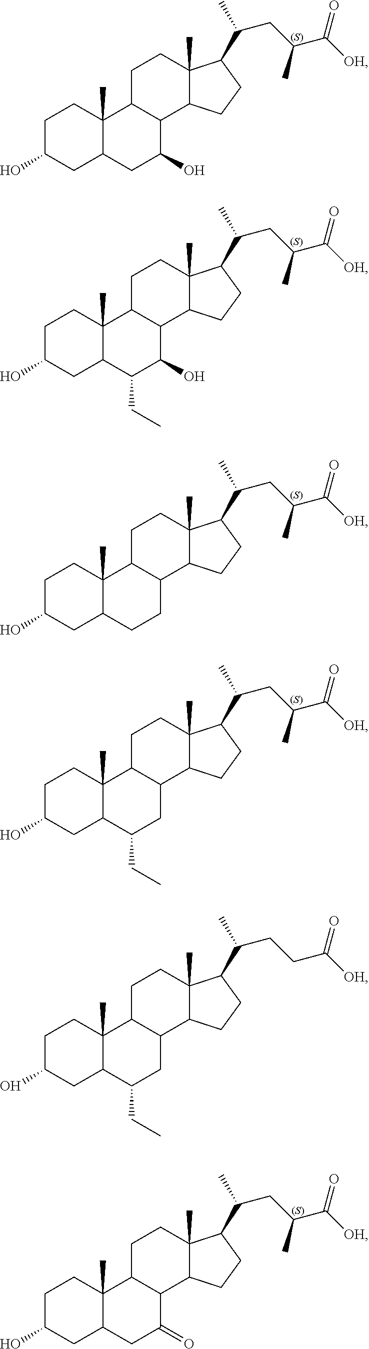

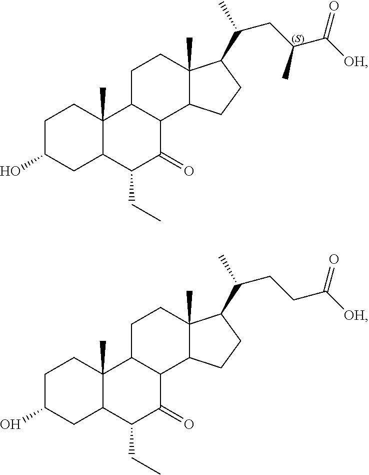

or a pharmaceutically acceptable salt thereof.

In embodiments, the compounds herein (e.g. of formula (I), (II), (III), (IV), or (V)) including embodiments thereof) has formula:

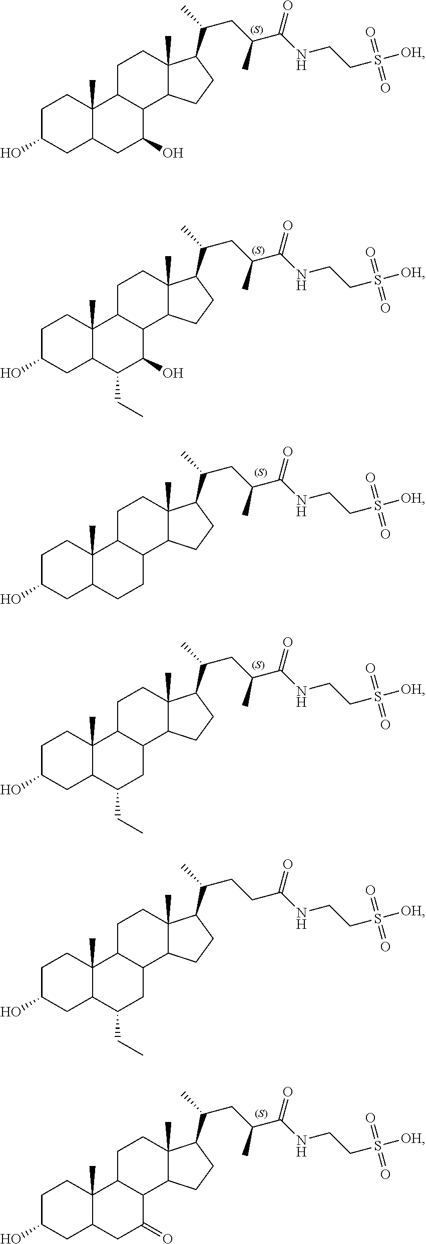

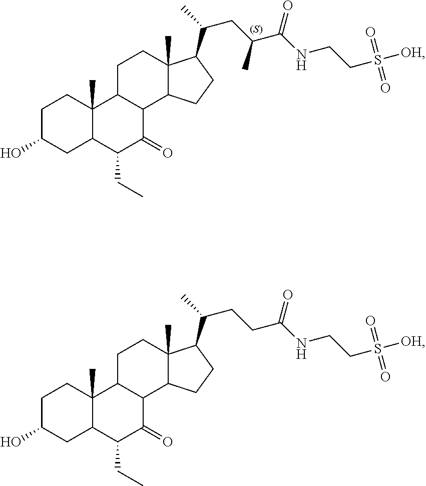

or a pharmaceutically acceptable salt thereof.

In embodiments, the compounds herein (e.g. of formula (I), (II), (III), (IV), or (V)) including embodiments thereof) has formula:

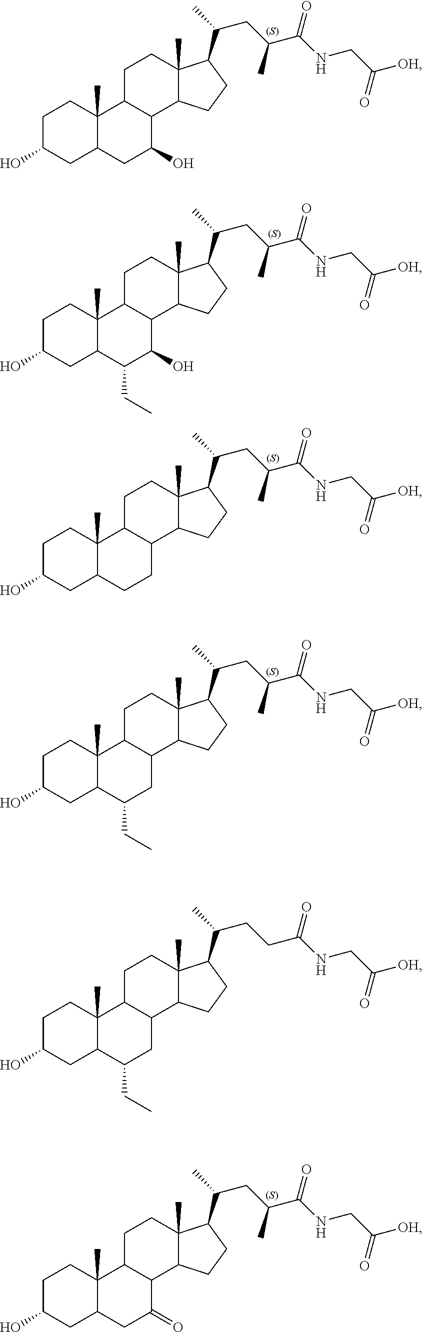

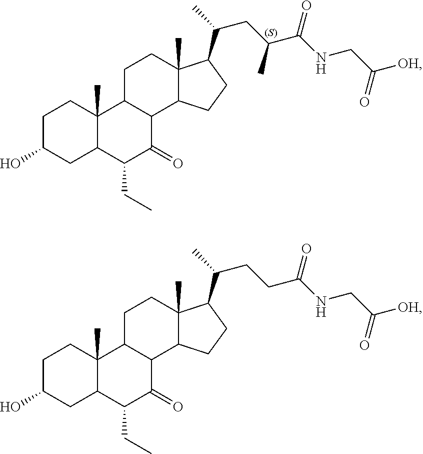

or a pharmaceutically acceptable salt thereof.

In embodiments, the compounds herein (e.g. of formula (I), (II), (III), or (IV), including embodiments thereof) has formula:

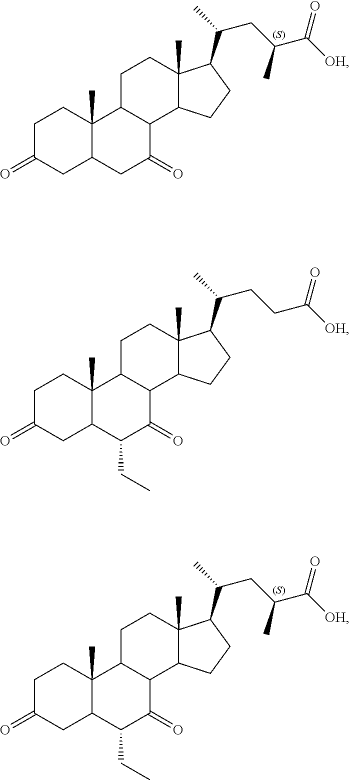

or a pharmaceutically acceptable salt thereof.

In embodiments, the compounds herein (e.g. of formula (I), (II), (III), (IV), or (V)) including embodiments thereof) has formula:

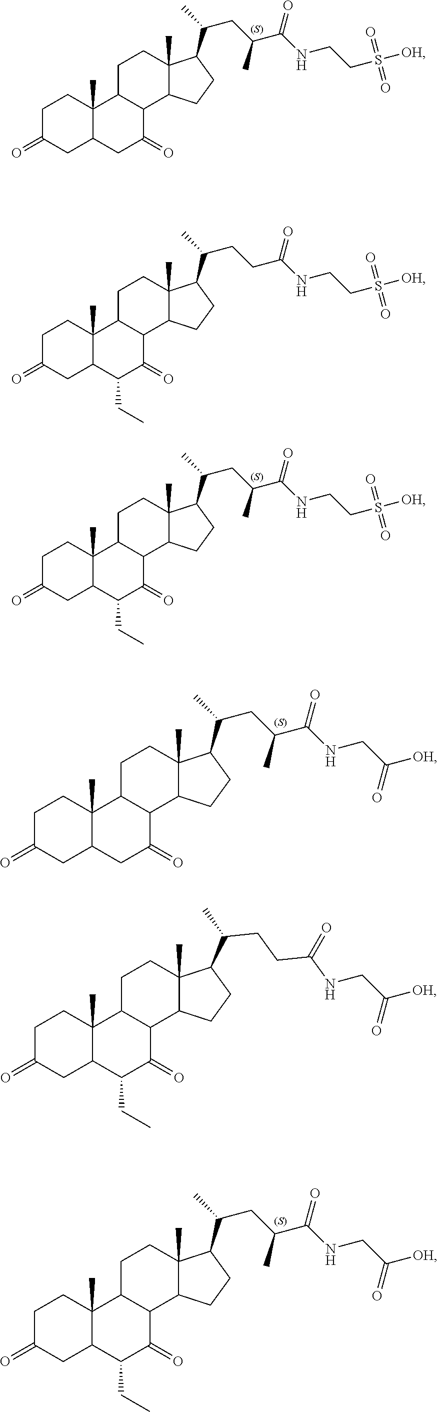

or a pharmaceutically acceptable salt thereof.

In another aspect is provided a pharmaceutical composition including a pharmaceutically acceptable excipient and a compound as provided herein (e.g. formula (I), (II), (III), (IV), (V), (VI), (VII), or a compound of Table 1), including embodiments thereof.

### III. Methods

In another aspect is a method of synthesizing a compound of formula (I). The method includes contacting a compound of formula (II) with an alkylating agent in the presence of a sterically hindered base. The method further includes contacting the compound of Formula (II) with a carboxylate deprotecting agent, thereby synthesizing a compound of Formula (I). The carboxylate deprotecting agent is as described herein, including embodiments thereof. In embodiments, one or more of R¹, R², R³, R⁵, R⁶, R⁷, R⁸, R⁹, R¹⁰, R¹¹, R¹², R¹³, R¹⁴, and R¹⁵ of the compound of formula (II) is respectively —OR^{1A}, —OR^{2A}, —OR^{3A}, —OR^{5A}, —OR^{6A}, —OR^{7A}, —OR^{8A}, —OR^{9A}, —OR^{10A}, —OR^{11A}, —OR^{12A}, —OR^{13A}, —OR^{14A}, —OR^{15A}. R^{1A}, R^{2A}, R^{3A}, R^{5A}, R^{6A}, R^{7A}, R^{8A}, R^{9A}, R^{10A}, R^{11A}, R^{12A}, R^{13A}, R^{14A} and R^{15A} may independently an alcohol protecting group. The method may further include contacting the compound with an alcohol deprotecting agent as described herein, including embodiments thereof.

In embodiments, the carboxylate deprotecting agent is an element, a mild base, a strong base, an organometallic reagent, or an aqueous acid and the removal (i.e. deprotection) may be performed techniques understood by those skilled in the art. The carboxylate deprotecting agent may be LiOH, diethyl amine, triethyl amine, piperidine, tetrabutylammonium hydroxide, fluoride ion, hydrogenation, or sodium, in a solvent using techniques understood by those skilled in the art. In embodiments, after removing the carboxylate protecting group, the compound is reacted with a compound having the formula H₂N—R^{16A}, as described herein.

The alcohol protecting groups may independently be alkyl ethers, aryl ethers or silyl ethers. In embodiments, the alcohol protecting groups are allyl, benzyl, PMB or trityl groups. In embodiments, the alcohol protecting groups are TMS, TES, TIPS, TBDMS or TBDPS groups. The alcohol protecting groups may independently be esters or acetals. In embodiments the alcohol protecting groups are tetrahydropyranyl (THP) groups. In embodiments, the alcohol protecting groups are MOM, MEM MOP or EE groups. In embodiments, the alcohol protecting groups are acetate, benzoate, or Troc groups.

The alcohol deprotecting agent may be an element, a mild base, a strong base, a mild acid, an aqueous acid, a lewis acid or an organometallic reagent and deprotection may be performed using techniques understood by those skilled in the art. In embodiments, the alcohol deprotecting agent may be zinc bromide, magnesium bromide, titanium tetrachloride, dimethylboron bromide, or trimethylsilyl iodide. In embodiments, the alcohol deprotecting agent is a silver (Ag⁺) or mercury (Hg⁺) salt. In embodiments, the alcohol deprotecting agent is zinc, samarium diiodide or sodium amalgam. In embodiments, the alcohol deprotecting agent is trifluoroacetic acid, hydrofluoric acid, or hydrochloric acid. The alcohol deprotecting agent may be hydrogen or hydrogenation. In embodiments, the alcohol deprotecting agent may be tetra-n-butylammonium fluoride (TBAF), boron trifluoride, or silicon tetrafluoride. The alcohol deprotecting agent may be Pyridinium p-Toluenesulfonate (PPTS).

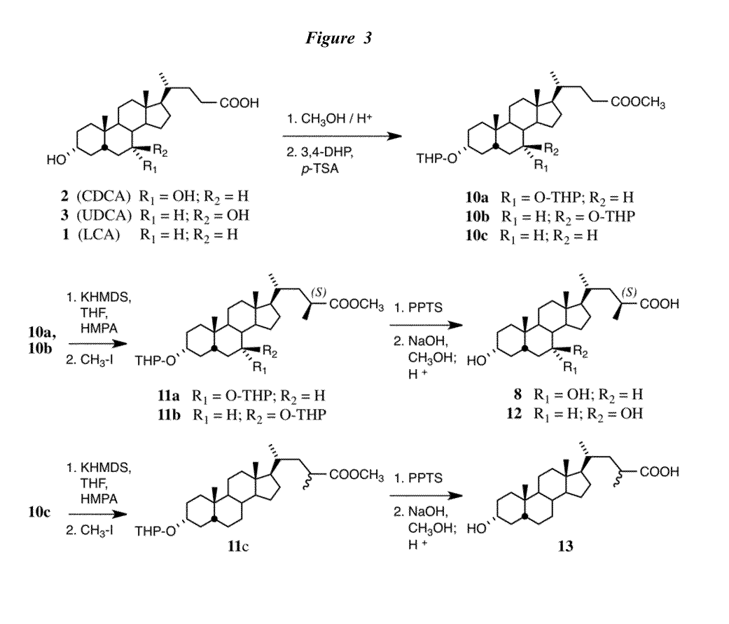

The alkylation may be stereospecific. Thus in embodiments, the alkylation may yield the (S) stereoisomer. In embodiments, the method of synthesizing the compound of formula (I) is as set forth in FIG. 3, 4, or 5.

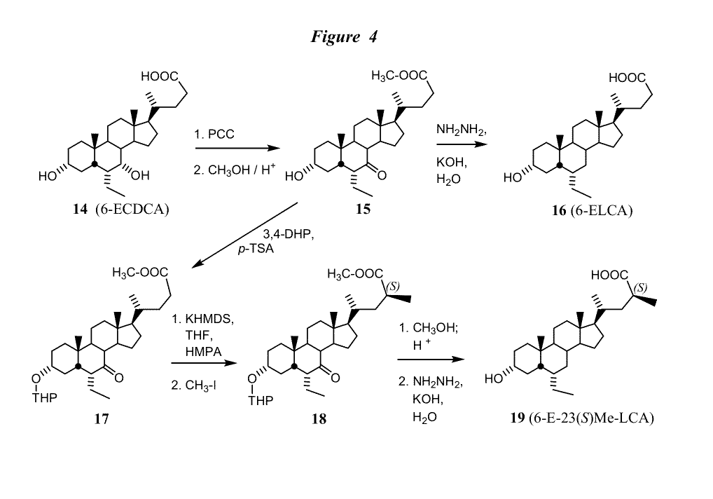

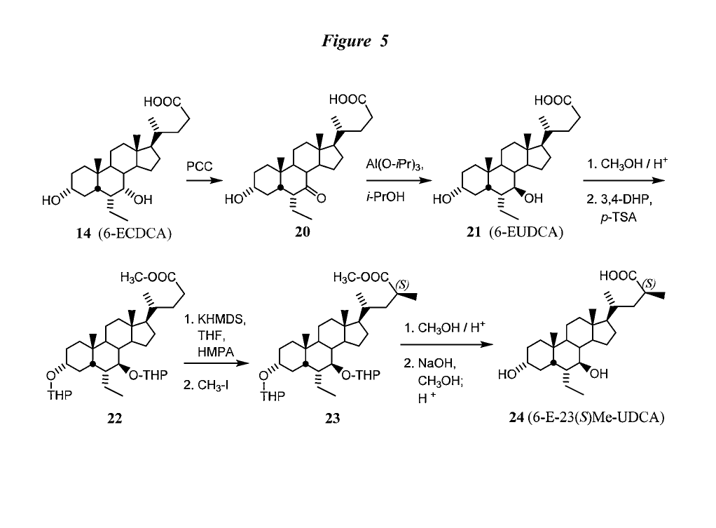

In embodiments, X¹ of the compound of formula (II) is —C(O). In embodiments, the method further includes contacting X¹ of the compound of formula (II) with a reducing agent to form a —CH₂ or —C(H)OH moiety. The reduction may result in X¹ of formula (I) being a —CH₂ moiety. The reduction may result in X¹ of formula (I) being a —C(H)OH moiety. In embodiments, X¹ of formula (II) is reduced as set forth in FIG. 4. X¹ of formula (I) may be —C(H)OH. In embodiments, X¹ of formula (II) is reduced as set forth in FIG. 5.

The reducing agent may be a compound or element that adds a hydrogen in catalytic hydrogenation reactions. In embodiments, the reducing agent is a transition metal. In embodiments, the reducing agent is platinum (Pt), palladium (Pd), rhodium (Rh), ruthenium (Ru) or nickel (Ni). The reducing agent may be a finely dispersed solid, adsorbed on an inert surface, or complexed with a soluble compound. The reducing agent may be a compound or element that transfers a hydride from a donor. The reducing agent may be a boron or aluminum hydride donor. Accordingly, the reducing agent may be LiAlH₄; LiAlH₂(OCH₂CH₂OCH₃)₂; LiAlH[(OC(CH₃)]₃; NaBH₄; NaBH₃CN; Ca(BH₄)₂; diborane (B₂H₆); or DIBAlH (diisobutylaluminium hydride). The reducing agent may be aluminum alkoxide. In embodiments, the reducing agent is a tri-alkyl or tri-aryl silane. The reducing agent may be triethyl silane or triphenyl silane. The reducing agent may be hydrazine.

In embodiments of the method, the alkylation agent used in the contacting step is an alkyl halide. The alkylation agent used in the contacting step may be an alkyliodide. The alkylation agent may be methyliodide.

The sterically hindered base may be (M⁺¹)HMDS, (M⁺)tBuO, (M⁺¹)TMP, (M⁺¹)PhO, (M⁺¹)MeO, (M⁺¹)EtO, DBU, Dabco, N,N-dichlorohexylmethylamine, N,N-diisopropyl-2-ethylbutylamine, 2,6-di-tert-butyl-4-methylpyridine, pentamethylpiperidine, MTBD, PMDBD, TBD, or tri-tert-butylpyridine. (M⁺¹) is Na, K, or Li. The sterically hindered base may be (M⁺¹)HMDS. (M⁺¹) is Na, K, or Li. In embodiments, the sterically hindered base is KHMDS. The contacting in the presence of a sterically hindered base may be performed as set forth in FIG. 3, 4, or 5.

The contacting may be performed in the presence of a polar aprotic solvent. In embodiments, the polar aprotic solvent is HMPA, HMPT, DMF, DMSO, MeCN, dioxane, methylpyrrolidone, DMPU, or a tetra-alkyl urea. The polar aprotic solvent may be HMPA, HMPT, DMF, or DMSO. The polar aprotic solvent may be HMPA. The contacting in the presence of a polar aprotic solvent may be performed as set forth in FIG. 3, 4 or 5.

In embodiments, the method includes oxidizing a compound of formula (I) when at least one of X¹ or X² is a —C(H)OH moiety. The at least one of X or X²—C(H)OH moiety is oxidized to —C(O). The oxidizing includes contacting the at least one X¹ or X²—C(H)OH moiety in the compound of formula (I) with an oxidizing agent. The oxidizing agent is allowed to oxidize the at least one X¹ or X²—C(H)OH moiety to —C(O). The oxidizing agent may be pyridinium chlorochromate (PCC).

In another aspect is a method of synthesizing a compound of formula (VII):

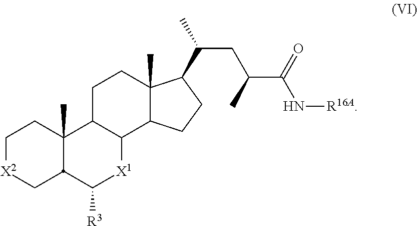

The method includes contacting a compound of formula (I), wherein R¹⁶ is —OH, with a compound having the formula, H₂N—R^{16A} (e.g. a free amine group attached to R^{16A} as described herein, including embodiments thereof) in the presence of a coupling reagent such as: EEDQ (N-ethoxy-2-ethoxy-1,2-dihydroquinoline); BOP (Benzotriazole-1-yl-oxy-tris-(dimethylamino)-phosphoniumhexafluorophospate); PyBOP (Benzotriazole-1-yl-oxy-tris-pyrrolidino-phosphonium hexafluorophosphate); DMAP (4-dimethylaminopyridine); HATU (2-(O-(7-azabenzotriazol-1-yl)-1,1,3,3-tetramethyluronium hexafluorophosphate); HBTU (2-(1H-benzotriazole-1-yl)-1,1,3,3-tetramethylaminium hexafluorophosphate); HCTU (2-(6-chloro-1H-benzotriazole-1-yl)-1,1,3,3-tetramethylaminium hexafluorophosphate; or HObt (N-hydroxybenzotriazole). In embodiments, the conjugation is done in the presence of EEDQ. In embodiments, R^{16A} is a taurine moiety (e.g. —CH₂CH₂SO₃H) or an amino acid moiety. The amino acid moiety may be a glycine moiety (e.g. —CH₂COOH). The compound of formula (I) may contacted with an amino acid according to scheme 1.

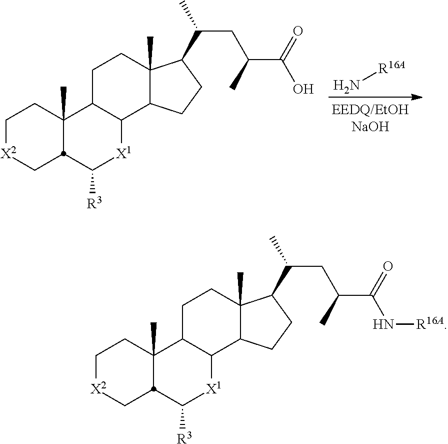

X¹, R¹, R², and R³ is as defined herein. R^{16A} may be (—CH₂CH₂SO₃H) or (—CH₂COOH) or any amino acid or a pharmaceutically acceptable salt thereof.

In another aspect is a method of synthesizing a compound of formula (VII):

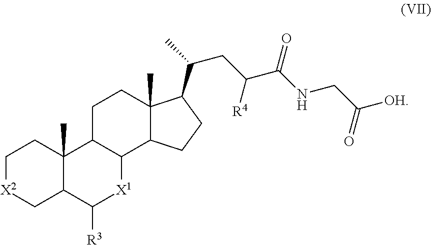

The method includes contacting a compound of formula (I) with a glycine moiety (e.g. —NHCH₂COOH) in the presence of a coupling reagent such as: EEDQ (N-ethoxy-2-ethoxy-1,2-dihydroquinoline); BOP (Benzotriazole-1-yl-oxy-tris-(dimethylamino)-phosphoniumhexafluorophospate); PyBOP (Benzotriazole-1-yl-oxy-tris-pyrrolidino-phosphonium hexafluorophosphate); DMAP (4-dimethylaminopyridine); HATU (2-(O-(7-azabenzotriazol-1-yl)-1,1,3,3-tetramethyluronium hexafluorophosphate); HBTU (2-(1H-benzotriazole-11-yl)-1,1,3,3-tetramethylaminium hexafluorophosphate); HCTU (2-(6-chloro-1H-benzotriazole-1-yl)-1,1,3,3-tetramethylaminium hexafluorophosphate; or HObt (N-hydroxybenzotriazole). In certain embodiments, the conjugation is done in the presence of EEDQ.

X¹, X², R³, and R⁴ are as described herein, including embodiments thereof.

In another aspect is a method of treating or preventing diabetes, obesity, insulin resistance, or liver disease in a subject in need thereof. The method includes administering to the subject a therapeutically effective amount of a compound as described herein (e.g. compound of formula (I), (II), (III), (IV), (V), (VI), (VII) or a compound of Table 1), including embodiments thereof. In embodiments, the method for treating includes administering to a subject in need thereof, a pharmaceutical composition of a compound as provided herein (e.g. formula (I), (II), (III), (IV), (V), (VI), (VII) or a compound of Table 1), including embodiments thereof. The pharmaceutical composition may include a pharmaceutically acceptable excipient. The administration may be performed intravenously or orally. The subject may be administered the compound for treating or preventing diabetes. In embodiments, the diabetes is type 2 (T2) diabetes. The subject may be administered the compound for treating or preventing obesity. The subject may be administered the compound for treating or preventing insulin resistance.

In another aspect is a method of treating or preventing cancer. The method includes administering to a subject in need thereof a therapeutically effective amount of a compound as described herein (e.g. compound of formula (I), (II), (III), (IV), (V), (VI), (VII) or a compound of Table 1), including embodiments thereof. In embodiments, cancer is liver cancer.

### IV. Examples

### Example 1

TGR5 (Takeda G-protein-coupled receptor 5) is a novel G-protein coupled receptor regulating various non-genomic functions via bile acid signaling that has emerged as a promising target for metabolic disorders, including obesity and type II diabetes. However, given that many bile acids (BAs) are poorly tolerated for systemic therapeutic use, there is significant impetus to develop TGR5 agonists with improved potency and specificity. There also is significant impetus to develop a stereoselective synthetic methodology for the TGR5 agonists. Here, we report the development of highly stereo-controlled strategy to investigate a series of naturally occurring bile acid derivatives with markedly enhanced TGR5 activity. These novel TGR5 agonists are evaluated in vitro by using luciferase-based reporter and cAMP assays to elucidate their biological properties. Additionally, computational modeling studies of selected ligands that exhibit enhanced potency and specificity for TGR5 are provided and discussed.

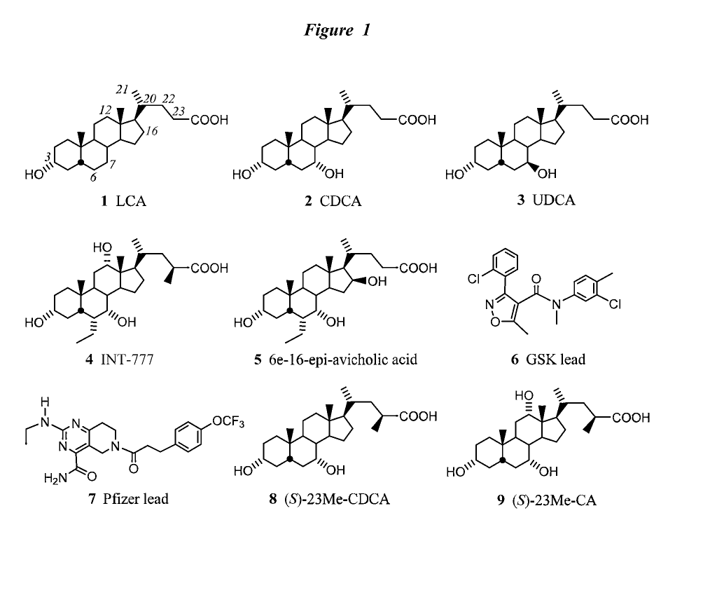

Naturally-occurring bile acids are the physiological ligands of TGR5.6 However, from a pharmacological perspective; endogenous bile acids are very weak TGR5 ligands in terms of both potency and specificity. Notably, bile acids not only activate TGR5, but also trigger activation of FXR, providing a significant obstacle for drug discovery efforts. Although the search for potent, selective TGR5 agonists has intensified, only a few agonists have been identified (FIG. 1).

In devising chemical modifications of the bile acid side chain and nucleus to afford more potent and selective TGR5 modulators, the role of stereochemistry on the specific biological activities of bile acids should be kept in mind. The absolute configuration of alkyl-substituted bile acid derivatives has a significant influence on specificity towards TGR5.7 Computational modeling studies indicate that TGR5 is endowed with a narrow binding pocket that preferentially recognizes the (S) configuration of 23-methyl-substituted bile acids, so that this stabilization of the ligand binding domain (LBD) predicts improved selectivity for TGR5.6 Discovered, inter alia, introduction of a methyl group at the C23(S) position of the natural bile acids CDCA to give 8 and cholic acid (CA) to give 9 confers selectivity for TGR5 over FXR activation, while the (R) epimers of these C23-methyl-substituted bile acids display very low activity and no specificity towards TGR5. This suggests that a minor stereochemical modification of bile acid side chain moieties, namely methylation at C23 to provide the (S) configuration, can improve their potency, affinity, selectivity, and bioavailability.

Relative to other bile acids, both LCA (1) and its C7-hydroxy epimer ursodeoxycholic acid (UDCA, 3) remain less explored. Among the naturally-occurring bile acids and their glycol derivatives, LCA is the most potent endogenous TGR5 agonist.¹¹ However, LCA is potentially toxic if used at high doses. UDCA has been approved by the FDA for the treatment of primary biliary cirrhosis (PBC), an autoimmune disease characterized by progressive cholestasis.¹² UDCA, in contrast to toxic hydrophobic bile acids, has also been shown to suppress the inflammatory response and, as such, is increasingly being employed for the treatment of hepatic and intestinal inflammatory diseases.¹² UDCA also binds FXR, presenting undesirable partial agonist activity of FXR.¹³ The presence of the equatorial 70-hydroxyl group in UDCA renders it more hydrophilic than CDCA (with its axial 7α-hydroxyl), and may contribute to UDCA's improved liver-function activity.¹⁴ However, like its other endogenous counterparts, UDCA possesses weak activity and low specificity. Indeed, UDCA doses at concentrations up to 200 μM have been shown to exert little-to-no effects on metabolic disease.

Stereoselective introduction of substituents α to a carbonyl can be readily achieved via alkylation using chiral auxiliaries or catalysts.¹⁶ However, the development of stereoselective syntheses to produce alkyl-substituted bile acid derivatives in high yields and few steps remains a formidable challenge. Taking advantage of three-dimensional quantitative structure-activity relationship (3D QSAR) studies that show how regions around the bile acid scaffold affect TGR5 activity,^{17,18} herein processes were designed that achieve stereoselective alkylation at the C23(S) position, and stereoselective Meerwein-Ponndorf-Verley reduction of the 7-keto group.

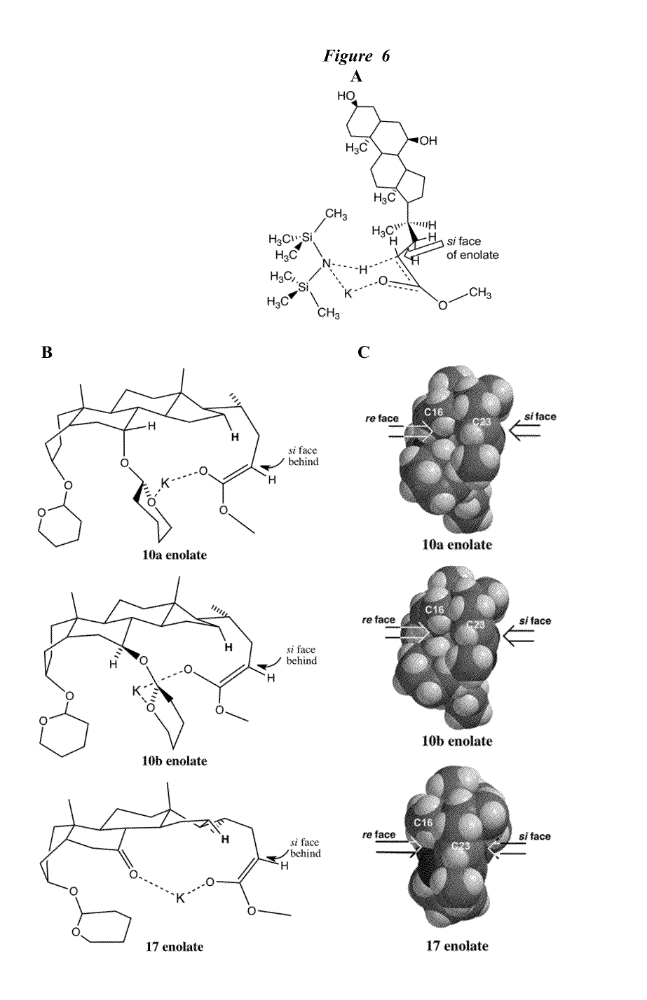

In attempting to devise a stereoselective strategy for the formation of 23(S)-methyl derivatives of LCA, CDCA, and UDCA, the use of the extremely bulky base potassium hexamethyldisilazide (KHMDS) was considered to form enolates of methyl esters of the parent acids. Recently, KHMDS has been used in the application of non-steroidal methylation of esters with methyliodide to generate an α-methyl product.¹³ KHMDS could permit stereocontrol during the deprotonation/methylation procedure in the steroid-ester because of its unique property of steric hindrance. Each ester's α carbon (C23) is two flexible bonds distant from the nearest stereocenter at C20. Thus, the bulky complex, shown in FIG. 6 during the deprotonation, could likely achieve adequate stereodifferentiation. In order to achieve 23(S) alkylation, the KHMDS complex should favor the re face of the αenolate carbon, exposing the si face for preferential attack on the alkylating agent iodomethane. In order for the bulky complex to control the face of attack, the KHMDS should not completely dissociate from the enolate after proton transfer, and should remain in proximity until alkylation occurs. Aa free enolate after dissociation would not be expected to show much preference for re or si attack, as shown by molecular mechanics calculations (MMFF, Spartan Student) indicating that re- and si-exposed uncomplexed conformations are within about a kcal/mol in steric energy of each other.

TABLE 1
Effects on TGR5 activity after C-23 (S) and C-6α modifications. HEK293
cells stably-expressing human TGR5 were treated with increasing doses of ligand after
6 hours of stimulation and intracellular cAMP levels were measured by luminescence.

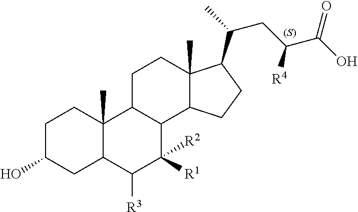

| | | | | | | TGR5 |
|---|---|---|---|---|---|---|
| | Compounds (Name) | R¹ | R² | R³ | R⁴ | EC₅₀, μM^{a} |
| 1 | LCA | H | H | H | H | 0.68 |
| 3 | UDCA | OH | H | H | H | 2.39 |
| 2 | CDCA | H | OH | H | H | 6.1 |
| | 5β-cholanic acid-3,7-dione | R₁═R₂═O | | H | H | 10.93 |
| 8 | 23(S)-Me-CDCA | H | OH | H | CH₃ | 2.83 |
| (S) | 23(S)-Me-LCA | H | H | H | CH₃ | 0.22 |
| 13 | | | | | | |
| 12 | 23 (S)-Me-UDCA | OH | H | H | CH₃ | 1.05 |
| 16 | 6α-E-LCA | H | H | CH₂CH₃ | H | 0.75 |
| 19 | 6α-E-23(S)-Me-LCA | H | H | CH₂CH₃ | CH₃ | 0.58 |
| 21 | 6α-E-UDCA | OH | H | CH₂CH₃ | H | 1.49 |
| 24 | 6α-E-23 (S)-Me-UDCA | OH | H | CH₂CH₃ | CH₃ | 0.73 |

| | | | | | |
|---|---|---|---|---|---|
| 27 | 23 (S)-Me-dione | R₁═R₂═O | H | CH₃ | 9.13 |

^{a}Data analysis to determine EC₅₀ values was performed with GraphPad Prism software using a cAMP standard curve and sigmoidal dose-response (variable slope) equation based on dose- response curves with tested compounds.

### Example 2

We designed a two-step procedure for the synthesis of 23(S)-methyl-substituted CDCA, UDCA, and LCA (FIG. 3). Acid-catalyzed esterification was performed with 1% sulfuric acid in methanol to obtain the corresponding methyl esters in quantitative yield. The hydroxyls of the crude methyl esters were then protected by treatment with dihydropyran and a catalytic amount of p-toluenesulfonic acid to give the corresponding tetrahydropyranyl (THP) ethers 10 in quantitative yields.

Treatment of the protected CDCA (10a) or UDCA (10b) with KHMDS in THF/HMPA at −78° C. produced enolates that reacted with the electrophile iodomethane. The THP groups were then removed by treatment with a catalytic amount of pyridinium p-toluenesulfonate (PPTS) (20 mol %),¹⁹ or an overnight reaction with 2N HCl in MeOH (25 mL).⁷ The methyl esters were subsequently hydrolyzed with NaOH in methanol at room temperature, affording the 23(S)-methyl-substituted CDCA (8) and new UDCA derivative (12). The crude products were purified by crystallization or in some case by flash chromatography eluted with CH₂Cl₂/MeOH (9:1).

It is worth noting that this methodology resulted in exclusive production of the 23-S-epimer (S/R>49:1). Thus, a two-step procedure of esterification/protection and deprotonation/methylation/deprotection/saponification sequence achieved the desired product (8), (12), and (13), in 65%, 10%, and 35% overall yields, respectively, starting from their native CDCA, UDCA, and LCA. The specific stereoselective synthesis of 23(S)-Me-CDCA, 23(S)-Me-LCA, and 23(S)-Me-UDCA is illustrated in FIG. 3.

The (S) and (R) epimers of 8 and 13 can be distinguished by the increased polarity of the (R) isomers on chromatography and by characteristic chemical shifts in the ¹H NMR spectrum (e.g. shifting of the C23 hydrogen). In the (S) isomers, this signal appears >2.45 ppm, whereas in (R) isomers it is <2.45 ppm. The methodology used herein resulted in exclusive production of the 23(S)-epimers of 8 and 12.

This sequence of esterification/protection followed by deprotonation/methylation and deprotection/saponification achieved the desired products 8, 12, and 13, in 65%, 10% and 35% overall yields, respectively, starting from the native CDCA, UDCA, and LCA.

In the presence of KHMDS and absence of HMPA, a reduced yield (<30%) and stereoselectivity (S:R=2:1) were observed. When HMPA was replaced by 18-crown-6, we observed reduced yield (17%) and stereoselectivity (S:R=1:3). Performing the reaction in KHMDS/toluene/HMPA led to a severely reduced yield (<10%). These experiments indicated that KHMDS/THF/HMPA was the best system for maintaining reaction efficiency and diastereoselectivity.

### Example 3

Alkyl groups at positions 23(S) and 6α were introduced to determine which modifications may significantly enhance binding affinity specificity towards TGR5. FIG. 4 describes syntheses of 6α-ethyl-LCA (16) and 6α-ethyl-23(S)-methyl-LCA (19). The synthetic strategy takes advantage of 6-ECDCA¹⁸ (14) as a starting material that allows access to a key ethylated intermediate, ketone 15. Reduction of 15 to the corresponding methylene compound by a modified Wolff-Kishner reaction²⁰ in ethylene glycol monomethyl ether gave the new LCA derivative 6α-ethyl-LCA (6-ELCA) 16 in 14% yield. After THP protection of the hydroxyl of 15, the stereoselective protocol described above (e.g. KHMDS/HMPA/THF/Me-I) was used for alkylation of the resulting 17, giving the desired 23(S)-Me intermediate 18. After deprotection, the modified Wolff-Kishner reduction gave the new LCA derivative 6α-ethyl-23(S)-methyl-LCA (19) in 12% yield after purification by flash column chromatography (Hexanes/EtOAc 1:1).

Following a methodology similar to that above, the ethylated CDCA 14 was oxidized by PCC to obtain the keto-acid 20 in 78% yield (FIG. 5). The keto group of 20 was then reduced stereoselectively by catalytic Meerwein-Ponndorf-Verley (MPV) reduction with aluminum alkoxide in iPrOH to give desired product 6α-ethyl-UDCA (21) in 21% overall yield. The stereoselective synthesis of 6α-ethyl-23(S)-methyl-UDCA (24) was similar to that of the corresponding unethylated derivative 23(S)-methyl-UDCA (12). Treatment of protected 22 with KHMDS/HMPA followed by iodomethane and then deprotection resulted in the installation of a methyl group at the 23(S) position to give desired new UDCA derivative 24 in 10% yield (FIG. 5).

The determination of the C7 stereochemistry of 6α-ethyl-UDCA (21) and its analogs was made by ¹H NMR. Compound 21 and 6α-ethyl-CDCA (14) are epimers, differing only in the configuration of the chiral carbon at C7. The C3 and C7 methine proton peaks of 14 and 21 are instantly identified. In 21, these protons are both in axial positions, and as such, they coincide at approximately 3.45 ppm.²¹ This is in contrast to the two distinct peaks (δ˜3.38, 3.78 ppm) caused by the protons of H-3β and H-7β, in equatorial and axial positions, respectively, observed in 14.¹⁸

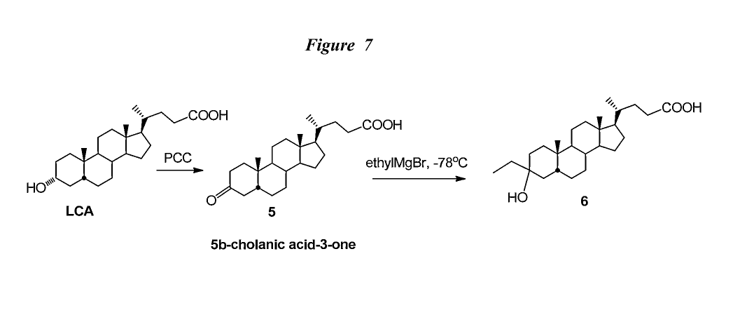

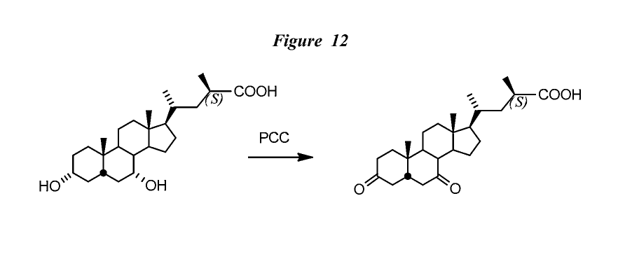

LCA modified by alkylation at the C3 position was also prepared using the methods described infra. PCC oxidation of 1 gave ketone 25, which was functionalized by a Grignard reaction to obtain ethylated 26 (FIG. 7). The oxidation of 8 with PCC gave the diketo 23(S)-methyl acid 27, a 5β-cholanic acid-3,7-dione derivative lacking C3 and C7 hydroxyl groups (FIG. 12).

### Example 4

TGR5 is a recently discovered bile acid cell surface receptor whose activation has emerged as a promising target for metabolic regulation and energy homeostasis. Thus the identification of novel TGR5 ligands would represent an important advance for drug discovery efforts targeting obesity and type II diabetes. Naturally occurring bile acids are weak agonists that activate both TGR5 and FXR, are poorly tolerated. As a result, these observations have hampered their use for clinical purposes. These obstacles underscore the need to develop more potent TGR5 modulators using stereoselective approaches.

The stereoselective alkylation of ester enolates is an extremely important organic reaction in medicinal chemistry. However, alkylation of steroidal esters in general bile acid synthesis has not attracted much attention as a stereoselective method. To overcome drawbacks to existing synthesis strategies and improve yields of alkyl substituted bile acid derivatives as potential TGR5 modulators, an efficient and highly stereoselective synthesis was developed, inter alia, of alkyl-substituted bile acids derivatives. For the insertion of alkyl groups on the 23(S)-position, a novel combination of the KHMDS/THF/HMPA/MeI system was employed, which predominately favored the formation of 23(S)-methyl-substituted bile acid derivatives in sufficient yields. It should be noted that stereoselective methylation of esters was observed in the procedure. Importantly, only the 23(S)-methyl substituted derivatives of CDCA, LCA, and UDCA were formed and negligible amounts of their 23(R)-methyl epimers were obtained. This stereoselective approach gave desired products of 23(S)-Me-CDCA (8), 23(S)-Me-LCA (13), 23(S)-Me-UDCA (12) in 65%, 35%, and 10% yields in total, respectively. Using a two-step procedure that starts with the naturally occurring bile acids CDCA, LCA, and UDCA by stereoselective alkylation to generate their novel derivatives for which was isolated as a single isomer with the methyl group in the S-position.

### Example 5

The key factors of this stereoselective synthesis incorporate the use of the bulky base KHMDS and solvent system THF/HMPA/MeI. To examine whether a difference in solvent composition would disturb the selectivity and reactivity, several combinations in solvent systems were tested. A reduced yield (<30% overall) and stereoselectivity (S/R=2:1) were observed when the reaction was conducted in the presence of KHMDS and absence of HMPA. When HMPA was replaced by 18-crown-6, (KHMDS/THF/18-crown-6), we observed reduced yield (17% overall) and less stereoselectivity (S/R=1:3). Performing the reaction in KHMDS/toluene/HMPA system led to severely reduced yield (<10% overall). These experiments indicated that the KHMDS/THF/HMPA/MeI system was the most efficient strategy at maintaining reaction efficiency and diastereoselectivity. This has been well exemplified by its application in the synthesis of 23(S)-methyl-CDCA (59-65% overall yield and S/R>49:1).

The role of HMPA is typically to dissociate complexes. Without being bound to any particular theory, if the complex shown in FIG. 6a is broken apart, the resulting free enolate should be prone to nonstereoselective electrophilic attack on either side. This appears to be the case when LDA is used as the base, resulting in S/R products in a 1:1 ratio,⁶ which should be asymmetric induction. Accordingly, and without being bound to any particular theory, in the presence of HMPA, KHMDS dissociates from the enolate, and the enolate undergoes an intramolecular complexation, bridged underneath the steroid nucleus by the potassium countercation, to a C7-associated oxygen atom. See FIG. 6b. There may be an alternative potassium-bridged complex around the front side of the steroid nucleus for 10a and 10b that preferentially exposes the re face of the enolate, but these complexes are much higher in steric energy, according to MMFF calculations.

Because of the asymmetric steroid nucleus, the two faces of each enolate complex have different environments. The re face of the enolate is hindered by the α hydrogen on C16, favoring attack on the si face, so that methylation exclusively gives the (S) product. FIG. 6c shows space-filling models looking straight down the plane of each enolate, with the two faces indicated by arrows. The si face, on the right side, is less hindered than the re face. The cholanic acid examples herein that give exclusive (S) methyl product have an oxygen at C7 to provide this sort of bridge. The LCA derivative 10c, lacking a C7 oxygen, forms an uncomplexed enolate that can be attacked from either side, causing nonstereoselectivity. In the absence of HMPA, the enolates can be stabilized by intermolecular complexation, affording diminished stereodifferentiation.

### Example 6

The preparation of the UDCA analogs required a stereoselective way to make 7β-OH 21 (6-EUDCA) from the 7-ketone 20. It is often hard to accurately control the opposing stereoelectronic and steric forces that come into play in such reductions. The 3α,7β,12α-bile acid can be prepared by sodium-alcohol reduction of its 7-keto analog. However, several attempts to perform this reduction starting from cholic acid were discouraging: low yields were obtained after a laborious, multistep process and separation of the resulting epimers and numerous byproducts by column chromatography.²⁴ The selective oxidation of 14 at C-7 to make 20 was also less straightforward than indicated in the literature.

The Meerwein-Ponndorf-Verley²⁷ (MPV) reduction using the bulky catalytic reducing agent Al(O-iPr)₃/i-PrOH worked well, giving 21 in 84% yield. The preparation of 21 has also been reported in a 2011 patent; however, that methodology was complicated, low yield, and restricted.²⁸ The current method is simple to perform and achieves higher yields in fewer synthetic steps. Aluminum isopropoxide is commercially available, but in our hands it proved less effective than dissolving the calculated amount of aluminum shavings in the appropriate amount of refluxing anhydrous isopropanol. The resulting solution of isopropoxide aluminum must be used immediately to achieve good catalytic activity and yield. This can be explained by the fact that crystalline Al(O-iPr)₃ is tetrameric, whereas in solution at the boiling point of iPrOH it is trimeric and more active.²⁹

In these experiments, aluminum isopropoxide was prepared in situ by dissolving the calculated amount of aluminum shavings in the appropriate amount of refluxing anhydrous isopropanol to form a fresh solution of isopropoxide aluminum. The resulting solution of isopropoxide aluminum should be used immediately to exhibit better catalytic activity for achieving good yield. This can be explained by the fact that crystalline Al(OiPr)₃ is tetramer, whereas in solution at the boiling point of i-PrOH it is trimeric and more active. Without being bound by any particular theory, the mechanistic details of the MPV reduction of ketones to the corresponding alcohols suggest that a direct hydrogen transfer mechanism involving a concerned six-membered ring transition state is the most favorable pathway.²⁵⁻²⁶ However, MPV reduction has not previously been used in stereospecific chemical techniques. As very limited examples of MPV reductions with reported stereoselectivities in steroidal synthesis have been reported, the stereoselective reduction on the substrate 6α-ethyl-7-keto (11) is in agreement with the traditional MPV mechanisms which preferably yields the more thermodynamically stable equatorial alcohol product.

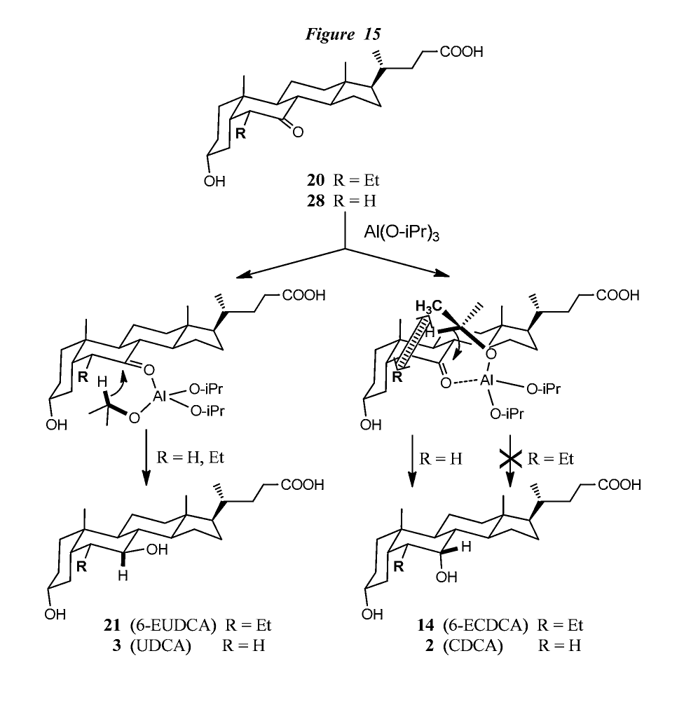

Given that no 6-ECDCA epimer 14 was observed in the reduction of 20 to 6-EUDCA 21, we were encouraged to produce UDCA (3) by the MPV reduction of 28¹⁸ (FIG. 15). Interestingly, the production of UDCA by MVP reduction is not completely stereoselective; a trace amount of CDCA (2) is formed. The stereoselectivity of the 6-EUDCA reaction does not seem to depend on the stability of the product alcohols (the β alcohol 21 is only 1 kcal/mol more stable than the α alcohol 14 by MMFF calculations; this is half the difference of 3 relative to 2) or of product alcohol complexes with Al(O-iPr)₃ (α has a lower steric energy than β). We propose that when hydride is delivered to the (3 face of the ketone, a methyl group on the isopropyl that provides the hydride has a steric interaction with the methylene on the C6 ethyl group. The raises the energy of the transition state going to α product relative to attack giving the β product. In the case of 28, there is no C6 ethyl group, so that steric penalty is absent, and both pathways can contribute.

### Example 7

The significance of the carboxylic acid of LCA as a contributor to the observed potency and selectivity in steroidal ligands for the TGR5 has also been observed. Reduction of the C-24-COOH into its corresponding CH₂OH of LCA resulted in a complete loss of activity. To investigate if the binding pocket of TGR5 would be endowed with narrow hydrogen bonding door site recognizing the 3-hydroxyl group of bile acids, the effects of LCA derivatives modified at the C-3 position were also examined. Alkyl modification of the C-3 position of the LCA nucleus (6) led to a large loss in potency, indicating that hydroxyl group in the 3α-position of LCA is required for TGR5 activity. The synthetic pathway of compound 6 is depicted in FIG. 7.

### Example 8

The possibility of hydrogen acceptor on 3,7-position of LCA was also investigated.

The oxidation of the 3α-OH and 7α-OH led to sharp decreases in activity, while introduction of a methyl group at the 23(S)-position of 3,7-diketo, enhanced the activity of TGR5 (17). These results suggest that the 23(S)-methyl group directs specificity towards TGR5.

### Example 9

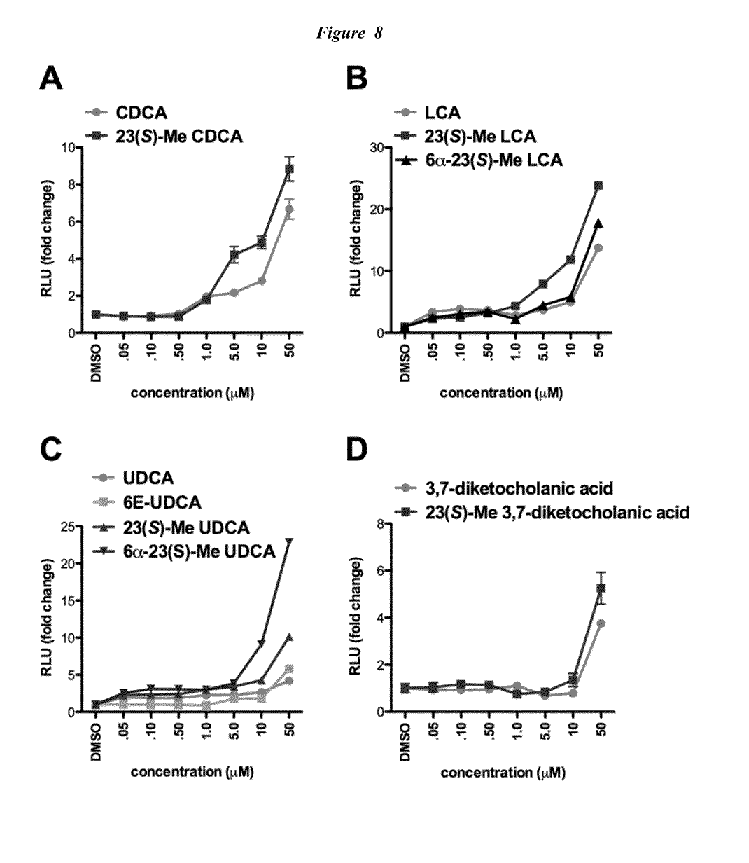

A series of luciferase reporter-based assays was performed to evaluate the biological effects of these novel bile acid derivatives. All compounds were evaluated in cell-based for in vitro potency and selectivity, using HEK293 cells stably-expressing human TGR5 transfected with the cAMP-sensitive reporter plasmid pCRE-Luc. Compounds were evaluated for their ability to activate TGR5 at 0.05, 0.1, 0.5, 1, 5.0, 10, and 50 μM test concentrations. All of the tested 23(S)-alkyl-substituted bile acid derivatives displayed markedly enhanced TGR5 specificity and potency relative to their native bile acids (FIG. 8A-D). In agreement with previous reports, these results suggest that 23(S)-methyl CDCA exhibits improved potency in luciferase assays. The biological activity of LCA, UDCA, and 3,7-diketocholic acid that were converted to the 23(S)-methyl series were assessed. Substituted LCA (3), UDCA (4) and 23(S)-Me-dione (17) all displayed increased potency compared to native LCA, UDCA, and 3,7-diketocholic acid. The most potent and specific TGR5 agonist observed was 23(S)-methyl-LCA (13) which improved LCA activity by almost two-fold at a 5 μM concentration.

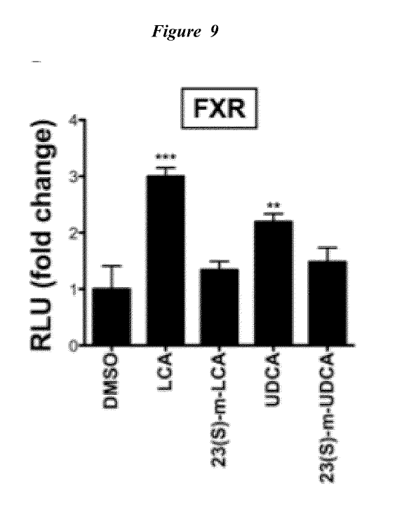

Similar luciferase assays were performed using HEK293 cells transfected with human FXR and an FXR-responsive reporter plasmid. See FIG. 9. No observable FXR luciferase activity was detected with methylated bile acids at doses up to 50 μM (FIG. 9), suggesting that introduction of the stereo alkyl side chain on the 23-position increases potency and selectivity on TGR5 over FXR. This experiment further shows that 23(S)-methyl-substituted LCA (3) itself, is a very effective TGR5 ligands. The 23(S)-substitution can even enhance the activity of 3,7-diketocholic acid (17), normally a precursor to bile acids with poor ligand-binding activities (FIG. 8D). Of the two positions for substitution on the bile acid system for influencing TGR5 potency and selectivity, the 6α-ethyl group on the steroidal B-ring has also been purported to be important for improving the potency of both TGR5 and FXR.⁸ However, 6α-ethyl modification improved the activity of UDCA but not LCA. UDCA is an FDA approved drug and related alternative medicine supplements and vitamins have already been on the USA market, thus raising the possibility of rapid translation of its potent derivatives to clinical use in the treatment of diabetes and obesity.

### Example 10

The changes in cAMP elicited by these bile acid derivatives was determined after binding to the TGR5. Structure-activity-relationship (SAR) of bile acid analogues 8, 12, (S)13, 16, 19, 21, 24, and 27 are described in Table 1. HEK293 cells stably expressing human TGR5 were treated with increasing doses of ligand after 6 hours of stimulation, and intracellular cAMP levels were measured by luminescence. As shown in Table 1, 23(S)-methyl-LCA (13), with an EC₅₀=0.21 μM has better activity and efficacy compared to the other tested bile acid derivatives. 6α-Ethyl-23(S)-Me-LCA (19) with an EC₅₀=0.58 μM has improved potency compared to the parent LCA. In the UDCA series, 23(S)-Me-UDCA (12) showed an EC₅₀ of 1.05 μM greater than the parent UDCA. 6α-E-23(S)-Me-UDCA (14) increased its agonist activity with an EC₅₀=0.71 compared to the UDCA. These results suggest that introduction of an alkyl group as S-form on the C-23 position contributes the biggest hydrophobic interactions or generates favorable steric constraints within the receptor TGR5 ligand binding pocket. These results are supported by molecular modeling studies showing that a pocket is formed by a specific network of hydrogen bonds which geometrically favors the (S) isomer over the (R) (FIG. 15).

The significance of the carboxylic acid group of LCA as a contributor to the potency and selectivity of steroidal ligands for TGR5 was examined. Reduction of the C24-COOH of LCA to the corresponding CH₂OH group resulted in a complete loss of activity. The importance of hydrogen-bond donors on the C3 and C7 positions of bile acids were also investigated. Conversion of these groups (CDCA) to ketones (5β-cholanic acid-3,7-dione) led to a sharp decrease in activity (EC₅₀ of 10.93, Table 1), while introduction of a methyl group at the 23(S)-position to give the diketone 27 (FIG. 12) enhanced the TGR5 activity (EC50 of 9.13). These results demonstrate once again that the 23(S)-methyl group directs specificity towards TGR5.

### Example 11

Among potential chemical templates for the development of novel TGR5 agonists, the steroidal bile acid core is unique. The co-crystal structure of ligand binding domain (LBD) complexed with the TGR5 has not been disclosed, however, knowledge-based or ligand-based approaches and homology modeling studies have been instrumental to depict features of the receptor binding site.^{32,33}

The binding of LCA based ligands to human TGR5 was examined using a three dimensional structural model of TGR5 using homology based computational methods. Two homology models of TGR5 were derived by using the SIP1R receptor and the active state J32-adrenergic receptor structure (pdb id: 3SN6) as templates. These templates used because S1PIR binds to an acid (phosphatidic acid) and, because an agonist is targeted, an active state model of TGR5 was needed. The homology models were refined using the LITiCon method to capture the sequence-specific structural aspects of TGR5.²⁷ The TGR5 structural models (FIG. 15) were validated by docking LCA and its derivatives and validating it against the existing SAR data.

The most potent TGR5 agonist synthesized was 23(S)-me-LCA (13) which displayed an EC₅₀ of 0.22 μM. Both 23(S)-me-LCA (13) and 23 (R)-me-LCA, were docked respectively. Docking was performed using the GLIDE application via the Maestro interface.²⁸ A docking box was defined encompassing the binding pocket and the upper region of the protein, and a constraint was employed to ensure that the ligand contacted one of the two arginines on extracellular loop 1 (EC1). The resulting docked conformations were clustered by root mean square deviation in coordinates (RMSD). Subsequently, side chain rotamer optimization of the residues was performed in the binding site using PRIME, for representative structure chosen from each cluster.²⁹ A docked pose was selected based on factors including calculated protein ligand interaction energy, conservative property of ligand interacting residues, density of population of RMSD cluster, similarity of binding to other crystallized proteins complexed with LCA, and ability to explain experimentally-determined SAR data.

### Example 12

The computational modeling studies indicate that 23(S)-me-LCA 13 can indeed dock in the TGR5 ligand binding domain (LBD). While its R epimer 23(R)-me-LCA 19 displayed a steric clash (FIG. 15). This is in agreement with the previously reported finding that introduction of the 23(S)-methyl group in CDCA leads to an enhancement of TGR5 activity.⁶

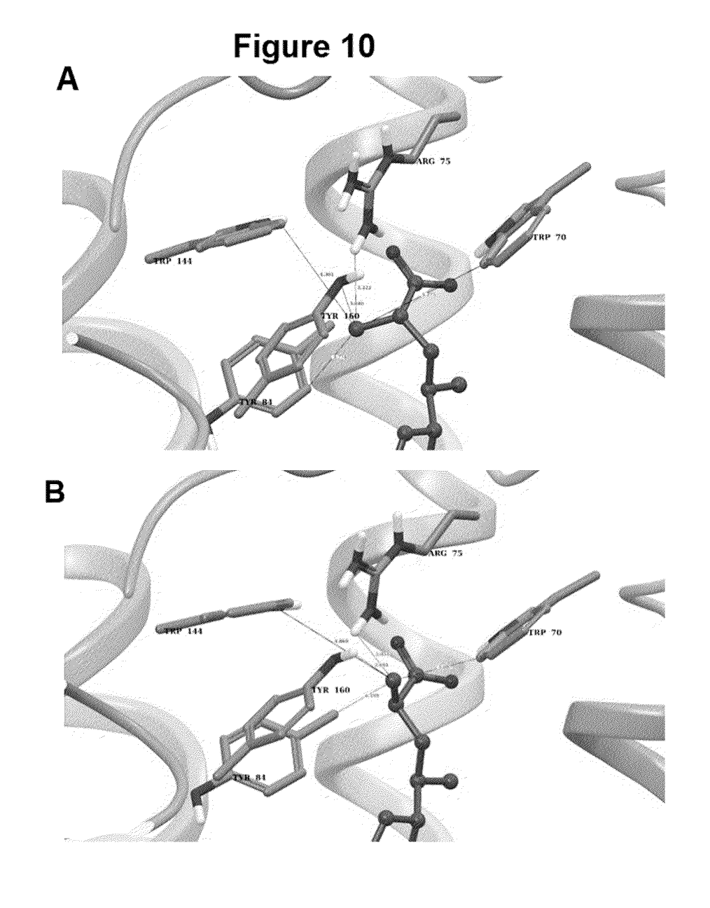

Our computational modeling studies present a schematic explanation for the increased efficacy of the 23(S) methyl: A pocket is formed by a specific network of hydrogen bonds so that 23(S)-Me-LCA (S) can indeed dock in the TGR5 ligand binding domain (FIG. 10A), while its (R) epimer 23(R)-Me-LCA (R) displayed a steric clash (FIG. 10B). This is in agreement with the previously reported finding that introduction of a 23(S) methyl group in CDCA leads to an enhancement of TGR5 activity.⁷ While this model does not place the water interactions that one would find in an experimental structure, it is an example of the most likely type of mechanism for the preference of the 23(S) methyl.

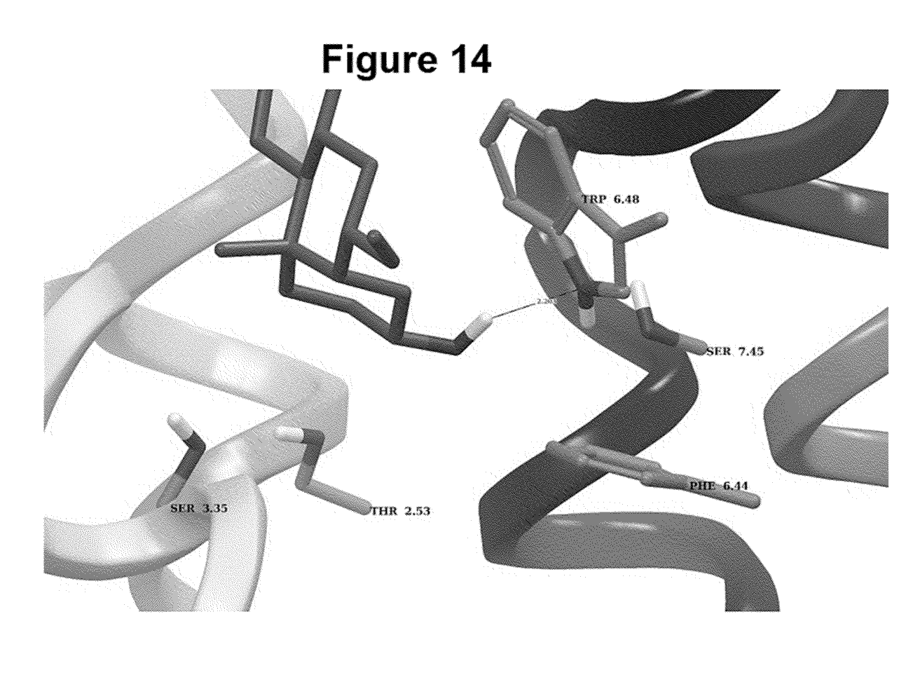

To investigate a narrow pocket with hydrogen-bond acceptor groups in 3α-OH of the LCA-TGR5 binding site and provide evidence to the study, we also docked new LCA derivative 6α-ethyl-23(S)-methyl-LCA (19) into the TGR516 pocket of the LBD of TGR5 (FIG. 14).

FIG. 14 revealed a narrow pocket with hydrogen-bond acceptor groups in 3α-OH of the LCA-TGR5 binding site. In addition to the interactions described above, the presence of a α-hydroxyl group at position 3 in LCA derivatives appeared to be critical for its TGR5 agonistic activity, mainly for the involvement of this OH group and 23(S) methyl group. It makes a hydrogen bond with the ring nitrogen of Trp 6.48. Depending on the rotation of the hydrogen and translation of the ligand, it may also interact with Thr 2.53 or Ser 3.35, however the geometry of the model indicates that either of these contacts are unlikely as the ligand ring bends away from TM3 and the binding site is distant from TM2.

### Example 13

### Methyl 3α,7α-Ditetrahydropyranyloxy-5-cholan-24-oate (10a)

To a solution of chenodeoxycholic acid (CDCA, 2) (1.0 g, 2.5 mmol) in methanol (25 mL) was added concentrated sulfuric acid (0.2 ml) and the reaction mixture was stirred at room temperature for 12 h. After concentration to remove methanol, the oil was dissolved in ethyl acetate (30 mL), washed with water (20 ml), saturated aqueous NaHCO₃ (30 ml), and brine (30 ml). The organic layer was dried over Na₂SO₄ and concentrated to give the methyl ester as a white solid (1.02 g). ¹H NMR (CDCl₃) δ 0.68 (s, 3H), 0.92 (s, 3H), 0.97 (d, 3H), 2.35 (m, 1H), 3.36 (s, 1H), 3.66 (s, 3H), 3.78 (s, 1H). The crude methyl 3α,7α-dihydroxy-5β-cholan-24-oate was subsequently dissolved in dioxane (5 mL), and p-toluenesulfonic acid (0.05 g, 0.26 mmol) and 3,4-dihydro-2H-pyrane (2 mL, 23.77 mmol) were added. The reaction mixture was stirred at room temperature for 30 min and checked by TLC. H₂O (25 mL) was then added and the mixture was partially concentrated under vacuum and extracted with EtOAc (3×30 mL). The combined organic fractions were washed with brine (1×30 mL), dried over Na₂SO₄ and evaporated under vacuum to give the crude product which was purified by flash column chromatography (EtOAc/Hexanes 8:2) to give a desired product as a colorless oil (1.31 g in 92% yield). ¹H NMR (CDCl₃) δ 4.47 (m, 2H), 3.94 (m, 2H), 3.74 (m, 2H), 3.47 (m, 2H), 0.92 (m, 6H), 0.63 (s, 3H). ¹³C NMR (CDCl₃) δ 174.7, 101.6, 101.5, 96.9, 96.6, 75.6, 62.8, 62.5, 55.5, 55.4, 51.4, 50.4, 49.7, 42.6, 41.9, 41.6, 39.7, 39.2, 37.1, 35.6, 33.5, 33.4, 32.8, 32.7, 31.3, 28.1, 23.5, 22.7, 20.5, 18.4, 18.1, 12.1.

### Methyl 3α-tetrahydropyranyloxy-5-cholan-24-oate (10c)

To a solution of lithocholic acid (LCA, 1) (1.0 g, 2.65 mmol) in methanol (25 mL) was added concentrated sulfuric acid (0.2 ml) and the reaction mixture stirred at room temperature for 12 h. After concentration to remove methanol, the oil was dissolved in ethyl acetate (30 mL), washed with water (20 ml), saturated aqueous NaHCO₃ (30 ml), and brine (30 ml). The organic layer was dried over Na₂SO₄ and concentrated to give the methyl ester as a white solid (1.02 g, 98% yield), mp: 125.4° C. (lit.³¹ mp: 126-128° C.). ¹H NMR (CDCl₃) δ 3.68 (s, 3H), 3.63 (m, 1H), 2.35 (m, 1H), 2.23 (m, 1H), 0.91 (s, 3H), 0.89 (d, 3H), 0.64 (s, 3H). ¹³C NMR (CDCl₃) δ 176.3, 73.3, 57.9, 57.3, 52.9, 22.2, 19.6, 13.4. The crude methyl 3α-hydroxy-5β-cholan-24-oate was subsequently dissolved in dioxane (5 mL), and p-toluenesulfonic acid (0.05 g, 0.26 mmol) and 3,4-dihydro-2H-pyrane (2 mL, 23.77 mmol) were added. The reaction mixture was stirred at room temperature for 1 h and checked by TLC. H₂O (25 mL) was then added and the mixture was partially concentrated under vacuum and extracted with EtOAc (3×30 mL). The combined organic fractions were washed with brine (1×30 mL), dried over Na₂SO₄ and evaporated under vacuum to give the crude product which was purified by flash column chromatography (EtOAc/Hexanes 8:2) to give the desired product, a colorless oil, as mixture of diastereoisomers (1.15 g, 92% yield). ¹H NMR (CDCl₃): (selected data) δ 4.95 (brs, 1H), 4.72 (brs, 1H), 3.92 (m, 2H), 3.66 (s, 3H), 3.63 (m, 1H), 3.51 (m, 2H), 2.36 (m, 1H), 2.23 (m, 1H), 0.91 (s, 3H). 0.90 (d, 3H), 0.63 (s, 3H). ¹³C NMR (CDCl₃) δ 176.2, 98.3, 98.0, 96.1, 77.3, 64.4, 57.8, 57.3, 53.0, 21.2, 19.5, 13.6.

### Methyl 3α-7β-tetrahydropyranyloxy-5β-cholan-24-oate (10b)

To a solution of UDCA (3, 0.5 g, 1.27 mmol) in methanol (15 mL) was added concentrated sulfuric acid (0.1 ml) and the reaction mixture stirred at room temperature for 12 h. After concentration to remove methanol, the oil was dissolved in ethyl acetate (30 mL), washed with water (20 ml), saturated aqueous NaHCO₃ (30 ml), and brine (30 ml). The organic layer was dried over Na₂SO₄ and concentrated to give the UDCA methyl ester as a semi-solid 0.5 g. ¹H NMR (CDCl₃): δ 3.67 (s, 3H), 3.59 (brs, 2H), 2.35 (m, 1H), 2.22 (m, 1H), 2.01 (m, 1H), 0.95 (s, 3H), 0.94 (d, 3H), 0.93 (t, 5H), 0.67 (s, 3H). The crude UDCA methyl ester was subsequently dissolved in dioxane (5 mL), and p-toluenesulfonic acid (0.05 g, 0.26 mmol) and 3,4-dihydro-2H-pyrane (2 mL, 23.77 mmol) were added. The reaction mixture was stirred at room temperature for 1 h and checked by TLC. H₂O (25 mL) was then added and the mixture was partially concentrated under vacuum and extracted with EtOAc (3×30 mL). The combined organic fractions were washed with brine (1×30 mL), dried over Na₂SO₄ and evaporated under vacuum to give the crude which was purified by flash column chromatography (EtOAc/Hexanes 7:3) to give a desired product as mixture of diastereoisomers and as a colorless oil (0.64 g, 90% yield). ¹H NMR (CDCl₃): (selected data) δ 4.71 (brs, 1H), 4.52 (q, 1H), 3.91 (m, 2H), 3.67 (s, 3H), 3.63 (m, 1H), 3.48 (m, 2H), 3.40 (m, 1H), 2.35 (m, 1H), 2.23 (m, 1H), 0.93 (s, 3H). 0.92 (d, 3H), 0.91 (m, 5H), 0.66 (s, 3H).

### 23(S)-Methyl-3α,7α-dihydroxy-5β-cholan-24-oic acid (8)

KHMDS (5 mL, 5 mmol, 1.0 M in THF) was added dropwise at −78° C. to a solution of 3α,7α-methyl ditetrahydropyranyloxy-5β-cholan-24-oate 10a (1.0 g, 1.73 mmol) and HMPA (1.82 g, 10 mmol) in dry THF (30 mL). The system was kept at −78° C. for an additional 30 min. After 30 min, methyl iodide (2.4 g, 17 mmol) dissolved in dry THF (3 mL) was slowly added. After 4-5 hours, an additional equivalent of KHMDS and methyl iodide were added if the starting material was not consumed (TLC), and the mixture was allowed to warm to room temperature overnight. The solvents were removed under vacuum and acidified by 10% HCl and extracted with EtOAc (3×30 mL), washed with 5% Na₂S₂O₃ solution (2×30 mL), dried over anhydrous Na₂SO₄, filtered, and evaporated under vacuum. The crude residue was treated with PPTS (0.01 g, 0.39 mmol) in MeOH (5 ml) for 7 h at 55° C., or a solution of 2N HCl in MeOH (25 mL) overnight. The solvent was evaporated and taken up with EtOAc, washed with a saturated NaHCO₃, dried over Na₂SO₄ and concentrated. The crude oil was dissolved in methanol (10 mL) and treated with 2N NaOH (8 mL) and the mixture stirred overnight at ambient temperature. After concentration to dryness, the residue was dissolved in EtOAc (25 mL) and water was added (10 mL); the solution was acidified to pH 3 with concentrated hydrochloric acid, then extracted into EtOAc (3×25 mL). The combined organic extracts were washed with brine and dried over Na₂SO₄, and then concentrated in vacuum to provide the carboxylic acid as semi-solid (a single 23(S)-diastereomer by NMR comparison) which was further purified by flash column chromatography (CH₂Cl₂/MeOH 9:1) to give the desired product as a white solid in 67% yield (0.46 g), mp: 136.1° C. (lit.⁶ mp: 125-126° C.). ¹H NMR (CDCl₃+CD₃OD) δ 3.77 (brs, 1H), 3.34 (m, 1H), 2.51 (m, 1H), 1.11 (d, 3H), 0.89 (d, 3H), 0.84 (s, 3H), 0.59 (s, 3H). ¹³C NMR (CDCl₃+CD₃OD) δ 179.9, 71.6, 68.1, 56.9, 50.6, 42.5, 41.5, 40.8, 39.6, 39.2, 37.1, 35.2, 34.9, 34.6, 34.5, 32.7, 30.1, 28.1, 23.5, 22.6, 20.5, 18.8, 18.1, 11.6. Anal. Calcd for C₂₅H₄₂O₄.H₂O: C, 70.71; H, 9.97. Found: C, 70.85; H, 9.88.

### 23(S)-Methyl-3α-hydroxy-5β-cholan-24-oic acid (13)

KHMDS (10 mL, 10 mmol, 1.0 M in THF) was added dropwise at −78° C. to a solution of methyl 3α-tetrahydropyranyloxy-5β-cholan-24-oate 10c (1.0 g, 2.11 mmol) and HMPA (1.82 g, 10 mmol) in dry THF (30 mL). The system was kept at −78° C. for additional 60 min. After 60 min methyl iodide (2.4 g, 17 mmol) dissolved in dry THF (3 mL) was slowly added. After 4-5 hours, an additional equivalent of KHMDS and methyl iodide were added if the starting material was not consumed (TLC), and the mixture was allowed to warm to room temperature overnight. The solvents were removed under vacuum, and the resulting residue was diluted with H₂O and acidified by 10% HCl and extracted with EtOAc (3×30 mL), washed with 5% Na₂S₂O₃ solution (2×30 mL), dried over anhydrous Na₂SO₄, filtered, and evaporated under vacuum. The crude residue was treated with PPTS (0.045 g, 0.18 mmol) in MeOH (5 ml), for 7 h at 55° C., or a solution of 2N HCl in MeOH (25 mL) for overnight. The solvent was evaporated and taken up with EtOAc, washed with a saturated NaHCO₃, dried over Na₂SO₄ and concentrated. The crude oil was dissolved in methanol (10 mL) and treated with 2N NaOH (8 mL) and the mixture stirred overnight at ambient temperature. After concentration to dryness, the residue was dissolved in EtOAc (25 mL) and water was added (10 mL); the solution was acidified to pH 3 with concentrated hydrochloric acid, then extracted into EtOAc (3×25 mL). The combined organic extracts were washed with brine and dried over Na₂SO₄, and then concentrated in vacuum to provide the carboxylic acid as semi-solid as a mixture of two compounds, which were separated by flash chromatography (Hexanes/EtOAc 1:1) into a less polar acid, 23(S)-methyl LCA derivatives (S)13, and a more polar acid, the 23(R)-methyl LCA derivative. The desired product (S)₁₋₃ is a white solid, 0.41 g (35% yield), mp: 176.8° C., ¹H NMR (CDCl₃): (selected data) δ 3.63 (brs, 1H), 2.60 (brs, 1H), 1.95 (m, 1H), 1.11 (d, 1H), 1.18 (d, 3H), 0.95 (d, 3H), 0.92 (s, 3H), 0.64 (s, 3H). ¹³C NMR (CDCl₃) δ 182.6, 73.3, 58.2, 57.9, 43.5, 42.2, 41.8, 41.6, 38.2, 37.8, 36.7, 36.1, 35.9, 31.9, 29.6, 28.6, 27.8, 25.6, 24.7, 22.2, 20.3, 20.0, 13.5. Anal. Calcd for C₂₅H₄₂O₃: C, 76.87; H, 10.84. Found: C, 76.38; H, 10.83.

### 3α,7α-Dihydroxy-23(S)-methyl-5β-cholan-24-oic acid (12)

KHMDS (10 mL, 10 mmol, 1.0 M in THF) was added dropwise at −78° C. to a solution of methyl 3α-7β-tetrahydropyranyloxy-5β-cholan-24-oate Ic (1.0 g, 1.82 mmol) and HMPA (1.82 g, 10.15 mmol) in dry THF (10 mL) under N₂ atmosphere. The system was kept at −78° C. for an additional 60 min. Iodomethane (3.87 g, 27.3 mmol) dissolved in dry THF (3 mL) was then slowly added. After 4-5 hours, an additional equivalent of KHMDS and methyl iodide were added if the starting material was not consumed (TLC), and the mixture was allowed to warm to room temperature overnight. The solvents were removed under vacuum, and the resulting residue was diluted with H₂O and extracted with EtOAc (3×30 mL), dried over anhydrous Na₂SO₄, filtered, and evaporated under vacuum. The crude residue was treated with PPTS (0.045 g, 0.18 mmol) in MeOH (5 ml), for 7 h at 55° C., or a solution of 2N HCl in MeOH (25 mL) for overnight. The solvent was evaporated and taken up with EtOAc, washed with a saturated NaHCO₃, dried over Na₂SO₄ and concentrated. The crude oil was dissolved in methanol (10 mL) and treated with 2N NaOH (8 mL) and the mixture stirred overnight at ambient temperature. After concentration to dryness, the residue was dissolved in EtOAc (25 mL) and water was added (10 mL), the solution acidified to pH 3 with concentrated hydrochloric acid, then extracted into EtOAc (3×25 mL). The combined organic extracts were washed with brine and dried over Na₂SO₄, and then concentrated in vacuum to provide the carboxylic acid as semi-solid single compound which was further purified by flash column chromatography (CDCl₃/MeOH 9:1) to give a desired product as a white solid 0.09 g in 20% yield, mp: 142.3° C. ¹H NMR (CDCl₃): (selected data) δ 3.59 (brs, 2H), 2.59 (brs, 1H), 2.01 (m, 1H), 1.16 (d, 3H), 0.97 (d, 3H), 0.96 (s, 3H), 0.92 (s, 3H), 0.66 (s, 3H). ¹³C NMR (CDCl₃) δ 183.1, 72.9, 72.8, 57.2, 57.1, 45.2, 45.1, 43.8, 42.4, 41.6, 40.6, 38.6, 38.5, 38.1, 36.3, 36.1, 35.4, 31.7, 30.1, 28.3, 24.8, 22.6, 20.2, 20.1, 13.5. Anal. Calcd for C₂₅H₄₂O₄. H₂O: C, 65.57; H, 9.59. Found: C, 65.61; H, 9.66.

### 3α-Hydroxy-6α-ethyl-7-keto-5β-cholan-24-oate (15)

To a suspension of chenodeoxycholic acid (14, 6ECDCA, 1.0 g, 2.37 mmol) and silica gel (4 g, 200-400 mesh, Aldrich) in anhydrous CDCl₃ (2 mL) was added, portionwise, pyridinium chlorochromate (PCC, 0.61 g, 2.8 mmol) in 25 mL of CH₂Cl₂ and the reaction mixture was stirred at room temperature for 15 min. The mixture was filtered and the filtrate was washed with water (20 mL) and brine (20 mL). The organic layer was dried over Na₂SO₄ and concentrated. The resulting crude oil was purified by flash column chromatography (CH₂Cl₂: MeOH 95:5) to afford the 7-keto acid as a solid (0.79 g, 79% yield), mp 201.1-201.7° C. ¹H NMR (500 MHz, CD₃OD) δ 3.50 (m, 1H), 2.94 (m, 1H), 2.52 (t, 1H), 2.30 (m, 2H), 2.19 (m, 6H), 1.70 (m, 2H), 1.43 (m, 4H), 1.31 (m, 6H), 1.19 (s, 3H), 1.12 (m, 4H), 0.92 (d, 3H), 0.67 (s, 3H). ¹³C NMR (125 MHz, CD₃OD) δ 213.7, 176.8, 70.1, 54.8, 49.2, 48.9, 47.7, 46.0, 44.9, 43.0, 42.4, 38.9, 36.8, 35.1, 34.9, 33.7, 31.0, 30.6, 29.2, 27.8, 24.3, 22.0, 21.4, 17.3, 10.5. Anal. Calcd for C₂₆H₄₂O₄: C, 74.60; H, 10.11. Found: C, 74.50; H, 10.13.

To a solution of the 7-keto acid (0.79 g, 1.89 mmol) in methanol (20 mL) was added concentrated sulfuric acid (0.1 ml) and the reaction mixture was stirred at room temperature for 12 h. After concentration to remove methanol, the oil was dissolved in ethyl acetate (30 mL), washed with water (20 ml), saturated aqueous NaHCO₃ (30 ml), and brine (30 ml). The organic layer was dried over Na₂SO₄ and concentrated to give the methyl ester 15 as a white solid (0.8 g). ¹H NMR (CDCl₃): (selected data) δ 3.64 (s, 3H), 3.56 (m, 1H), 2.82 (m, 1H), 2.33 (m, 2H), 2.19 (m, 2H), 1.17 (s, 3H), 0.92 (d, 3H), 0.90 (m, 5H), 0.63 (s, 3H). ¹³C NMR (CDCl₃) δ 213.9, 176.6, 72.2, 56.1, 52.9, 50.9, 50.3, 47.5, 46.8, 44.2, 40.3, 38.7, 36.6, 35.5, 32.4, 31.2, 29.6, 26.2, 24.4, 23.1, 19.7, 13.4.

Typical Procedure for Modified Wolff-Kishner Reduction: 6E-LCA (16).

A mixture of 0.1 g (0.23 mmol) of 3α-hydroxy-6α-ethyl-7-keto-5β-cholan-24-oic acid (15), KOH (0.4 g, 7.1 mmol), and 0.5 mL (10 mmol) of hydrazine monohydrate in 10 mL of ethyl glycol monomethyl ether and 0.3 mL H₂O was heated and stirred at 110° C. for 4 h and then refluxed for 8 h. The resulting mixture was cooled to 20° C., diluted with 5 mL of water and acidified to pH 2 with concentrated HCl. The precipitate was filtered off, washed five times with 10 mL of water, hexanes, and dried to give the desired product 16 as a white solid (0.04 g, 45% yield), mp 122.1° C. ¹H NMR (CDCl₃) (selected data) δ 3.56 (brs, 1H), 2.31 (m, 1H), 2.16 (m, 1H), 0.92 (m, 8H), 0.82 (s, 3H), 0.67 (s, 3H). ¹³C NMR (CDCl₃) (selected data) 8180.4, 73.3, 57.9, 57.3, 44.1, 43.5, 41.7, 40.1, 39.6, 35.3, 35.2, 33.1, 33.0, 31.0, 29.8, 27.8, 23.1, 22.3, 22.1, 20.5, 17.3, 12.4. Anal. Calcd for C₂₆H₄₄O₃.3H₂O: C, 68.07; H, 9.67. Found: C, 68.02; H, 9.67.

### Methyl 3α-tetrahydropyranyloxy-6α-ethyl-7-keto-5β-cholan-24-oate (17)

To a solution of 15 (0.5 g, 1.15 mmol) in 5 mL of dioxane were added p-toluenesulfonic acid (0.02 g, 0.12 mmol) and 3,4-dihydro-2H-pyrane (0.77 g, 9.2 mmol). The reaction mixture was stirred at room temperature for 60 min. Water (10 mL) was added and the mixture was extracted with EtOAc (3×30 ml); the combined organic layers were washed with saturated NaHCO₃ and brine and concentrated. The resulting crude oil was purified by flash column chromatography (Hexanes/EtOAc 7:3) to afford 17 as an oil (0.45 g, 75% yield). ¹H NMR (CDCl₃): (selected data) δ 4.73 (d, 1H), 3.86 (m, 1H), 3.76 (s, 3H), 3.59 (m, 1H), 3.46 (m, 1H), 2.82 (m, 1H), 1.17 (s, 3H), 0.92 (m, 8H), 0.63 (s, 3H). ¹³C NMR (CDCl₃) δ 213.3, 176.8, 96.4, 64.7, 62.1, 21.2, 21.1, 13.4.

### 3α-Hydroxy-6α-ethyl-23(S)-methyl-5β-cholan-24-oic acid (19)

KHMDS (10 mL, 10 mmol, 1.0 M in THF) was added dropwise at −78° C. to a solution of methyl 3α-tetrahydropyranyloxy-6α-ethyl-7-keto-5β-cholan-24-oate 17 (0.5 g, 0.97 mmol) and HMPA (0.91 g, 5.08 mmol) in dry THF (10 mL) under N₂ atmosphere. The system was kept to −78° C. for additional 60 min. Iodomethane (3.87 g, 27.3 mmol) dissolved in dry THF (3 mL) was then slowly added. After 4-5 hours, an additional equivalent of KHMDS and methyl iodide were added if the starting material was not consumed (TLC), and the mixture was allowed to warm to room temperature overnight. The solvents were removed under vacuum, and the resulting residue was diluted with H₂O and extracted with EtOAc (3×30 mL), dried over anhydrous Na₂SO₄, filtered, and evaporated under vacuum. The crude residue was treated with PPTS (0.045 g, 0.18 mmol) in MeOH (5 ml), for 7 h at 55° C., or a solution of 2N HCl in MeOH (25 mL) for overnight. The solvent was evaporated and the residue taken up with EtOAc, washed with saturated NaHCO₃, dried over Na₂SO₄ and concentrated to provide a crude oil. ¹H NMR (CDCl₃): (selected data) δ 3.64 (s, 3H), 3.51 (brs, 1H), 2.84 (m, 1H), 2.49 (m, 1H), 1.17 (s, 3H), 1.07 (d, 3H), 0.92 (m, 5H), 0.79 (d, 3H), 0.65 (s, 3H). ¹³C NMR (CDCl₃) δ 213.9, 176.6, 72.2, 56.1, 52.9, 50.9, 50.3, 47.5, 46.8, 44.2, 40.3, 38.7, 36.6, 35.5, 32.4, 31.2, 29.6, 26.2, 24.4, 23.1, 19.7, 13.4.

This intermediate was next reduced according to the typical procedure for modified Wolff-Kishner reduction. A mixture of 0.1 g (0.22 mmol) of 18, KOH (0.2 g, 3.55 mmol), and 0.25 mL (5 mmol) of hydrazine monohydrate in 5 mL of ethyl glycol monomethyl ether and 0.1 mL H₂O was heated and stirred at 110° C. for 4 h and then refluxed at 135° C. for 8 h. The resulting mixture was cooled to 20° C., diluted with water and acidified to pH 2 with concentrated HCl. The precipitate was filtered off, washed five times with 10 mL of water, hexanes, and dried to give compound 19 as a white solid (0.04 g, 45% yield), mp 137.7° C. ¹H NMR (CDCl₃) (selected data) δ 3.56 (brs, 1H), 2.51 (m, 1H), 1.12 (d, 3H), 0.91 (d, 3H), 0.90 (m, 5H), 0.87 (s, 3H). 0.69 (s, 3H). ¹³C NMR (CDCl₃) (selected data) 8181.2, 71.5, 55.3, 51.6, 50.9, 50.7, 50.6, 50.4, 45.8, 41.9, 39.6, 38.8, 36.1, 35.4, 31.1, 23.4, 22.2, 20.0, 13.6, 12.9. Anal. Calcd for C₂₇H₄₆O₃.H₂O: C, 73.44; H, 10.51. Found: C, 73.31; H, 10.84.

### 3α,7β-Dihydroxy-6α-ethyl-5β-cholan-24-oic acid (21)

To a solution of aluminum foil (0.1 g, 3.7 mmol) in anhydrous isopropanol (1.2 mL) was added mercuric chloride (0.005 g, 0.018 mmol). The reaction mixture was warmed up to reflux for 9 hours under an atmosphere of nitrogen until Al was completely dissolved. To this solution, 20 (0.05 g, 0.13 mmol) was added and the mixture was heated to reflux for 3.5 hours under N₂. After cooling, the reaction mixture was dissolved in EtOAc (10 mL) and water was added (15 mL), the solution was acidified to pH 2 with 2N hydrochloric acid, and then extracted with EtOAc (3×25 mL). The combined organic layers were washed with water and brine and concentrated to afford 6-EUDCA 21 as a semi-solid (0.042 g, 82% yield). Solidification by MeOH/CH₂Cl₂/pet ether gave a beige powder, mp 109.5° C. ¹H NMR (CDCl₃): δ 3.46 (brs, 1H), 3.45 (m, 1H), 2.35 (m, 1H), 2.22 (m, 2H), 0.93 (d, 5H), 0.89 (t, 3H), 0.83 (s, 3H), 0.67 (s, 3H). ¹³C NMR (CDCl₃) δ 178.8, 75.2, 71.9, 54.8, 51.5, 44.7, 44.2, 41.6, 40.4, 40.2, 39.5, 38.7, 36.2, 35.3, 34.3, 30.8, 30.7, 28.3, 27.3, 23.2, 22.8, 21.4, 18.4, 14.1. Anal. Calcd for C₂₆H₄₄O₄: C, 74.24; H, 10.54. Found: C, 74.00; H, 10.01.

### Methyl 3α,7β-tetrahydropyranyloxy-6α-ethyl-5β-cholan-24-oate (22)

To a solution of 21 (0.5 g, 1.27 mmol) in methanol (15 mL) was added concentrated sulfuric acid (0.1 ml) and the reaction mixture was stirred at room temperature for 12 h. After concentration to remove methanol, the oil was dissolved in ethyl acetate (30 mL), washed with water (20 ml), saturated aqueous NaHCO₃ (30 ml), and brine (30 ml). The organic layer was dried over Na₂SO₄ and concentrated to give the 6-EUDCA methyl ester as a semi-solid (0.5 g). ¹H NMR (CDCl₃): δ 3.67 (s, 3H), 3.59 (brs, 2H), 2.35 (m, 1H), 2.22 (m, 1H), 2.01 (m, 1H), 0.95 (s, 3H), 0.94 (d, 3H), 0.93 (t, 5H), 0.67 (s, 3H). The crude 6-EUDCA methyl ester was subsequently dissolved in dioxane (5 mL), and p-toluenesulfonic acid (0.05 g, 0.26 mmol) and 3,4-dihydro-2H-pyrane (2 mL, 23.77 mmol) were added. The reaction mixture was stirred at room temperature for 1 h and checked by TLC. H₂O (25 mL) was then added and the mixture was partially concentrated under vacuum and extracted with EtOAc (3×30 mL). The combined organic fractions were washed with brine (1×30 mL), dried over Na₂SO₄ and evaporated under vacuum to give a crude product that was purified by flash column chromatography (EtOAc/Hexanes 7:3) to give 22 as a mixture of diastereoisomers and as a colorless oil (0.64 g, 90% yield). ¹H NMR (CDCl₃): (selected data) δ 4.71 (brs, 1H), 4.52 (q, 1H), 3.91 (m, 2H), 3.67 (s, 3H), 3.63 (m, 1H), 3.48 (m, 2H), 3.40 (m, 1H), 2.35 (m, 1H), 2.23 (m, 1H), 0.93 (s, 3H). 0.92 (d, 3H), 0.91 (m, 5H), 0.66 (s, 3H).

### 3α,7β-Dihydroxy-23(S)-methyl-6α-ethyl-5β-cholan-24-oic acid (24)

KHMDS (1 mL, 1 mmol, 1.0 M in THF) was added dropwise at −78° C. to a solution of methyl 3α-7β-tetrahydropyranyloxy-5β-cholan-24-oate 22 (0.1 g, 0.18 mmol) and HMPA (0.18 g, 1.01 mmol) in dry THF (10 mL) under N₂ atmosphere. The system was kept at −78° C. for an additional 90 min. Iodomethane (0.38 g, 2.73 mmol) dissolved in dry THF (1 mL) was then slowly added. After 4-5 hours, an additional equivalent of KHMDS and methyl iodide were added if the starting material was not consumed (TLC), and the mixture was allowed to warm to room temperature overnight. The solvents were removed under vacuum, and the resulting residue was diluted with H₂O and extracted with EtOAc (3×10 mL), dried over anhydrous Na₂SO₄, filtered, and evaporated under vacuum. The crude residue was treated with PPTS (0.0045 g, 0.018 mmol) in MeOH (5 ml), for 7 h at 55° C., or a solution of 2N HCl in MeOH (15 mL) for overnight. The solvent was evaporated and taken up with EtOAc, washed with a saturated NaHCO₃, dried over Na₂SO₄ and concentrated. The crude oil was dissolved in methanol (10 mL) and treated with 2N NaOH (8 mL) and the mixture stirred overnight at ambient temperature. After concentration to dryness, the residue was dissolved in EtOAc (25 mL) and water was added (10 mL), the solution acidified to pH 3 with concentrated hydrochloric acid, then extracted into EtOAc (3×15 mL). The combined organic extracts were washed with brine and dried over Na₂SO₄, and then concentrated in vacuum to provide the carboxylic acid as a semi-solid which was further purified by flash column chromatography (Hexanes/EtOAc 1:1) to give a desired product as a white solid (0.01 g, 10% yield), mp: 106.6° C. ¹H NMR (CDCl₃): (selected data) δ 3.58 (brs, 2H), 2.58 (brs, 1H), 2.02 (d, 1H), 1.17 (d, 3H), 1.02 (m, 2H), 0.98 (t, 3H), 0.97 (d, 3H), 0.96 (s, 3H), 0.67 (s, 3H). ¹³C NMR (CDCl₃) δ 183.0, 72.8, 57.2, 57.1, 45.2, 45.1, 43.8, 41.6, 40.6, 38.6, 38.1, 36.3, 35.4, 31.7, 28.3, 24.8, 22.6, 20.3, 20.1, 13.5. Anal. Calcd for C₂₇H₄₆O₄. H₂O: C, 67.60; H, 9.96. Found: C, 67.72; H, 10.41.

### 5β-Cholanic acid-3-one (25)

To a suspension of lithocholic acid (1, LCA, 0.5 g, 1.32 mmol) and silica gel (2 g, 200-400 mesh, Aldrich) in anhydrous CHCl₃ (2 mL) was added, portionwise, pyridinium chlorochromate (PCC, 0.41 g, 19 mmol) in 25 mL of CH₂Cl₂ and the reaction mixture was stirred at room temperature for 60 min. The mixture was filtered and the filtrate was washed with water (20 mL) and brine (20 mL). The organic layer was dried over Na₂SO₄ and concentrated. The resulting crude oil was purified by flash column chromatography (CHCl₃: MeOH 9:1) to afford 513-Cholan-3-keto-24-oic acid as a solid (0.38 g, 81% yield), mp 137.7° C. (lit.²⁶ mp 140-143° C.). ¹H NMR (CDCl₃): (selected data) δ 2.71 (t, 1H), 2.41 (m, 1H), 2.30 (m, 2H), 2.17 (d, 1H), 1.10 (s, 3H), 0.93 (d, 3H), 0.69 (s, 3H). ¹³C NMR (CDCl₃) δ 215.0, 181.4, 57.8, 57.3, 45.7, 44.2, 43.7, 42.1, 41.4, 38.6, 38.4, 36.9, 36.7, 36.3, 32.3, 32.1, 29.5, 28.0, 27.1, 25.5, 24.1, 22.6, 19.6, 13.5.

### 3β-Ethyl-3α-hydroxy-LCA (26)

To a solution of 5β-cholanic acid-3-one (0.3 g, 0.77 mmol) in 30 mL of dry THF was added dropwise with stirring ethylmagnesium bromide (1 mL, 3 mmol, 3.0 M solution in diethyl ether) under argon at −78° C. The reaction mixture was stirred at −78° C. and allowed to warm overnight to room temperature, then quenched with 5% HCl. The aqueous phase was extracted with EtOAc, and the combined organic layers were washed by brine, dried over Na₂SO₄, filtered, and concentrated in vacuum. The crude product was purified by flash column chromatography (Hexanes/EtOAc 7:3) to afford the product 26 (0.23 g, 71%) as a white amorphous powder, mp: 90.9° C. ¹H NMR (CDCl₃): (selected data) δ 2.71 (t, 1H), 2.41 (m, 1H), 2.30 (m, 2H), 2.17 (d, 1H), 1.10 (s, 3H), 0.93 (d, 3H), 0.69 (s, 3H). ¹³C NMR (CDCl₃) δ 215.0, 181.4, 57.8, 57.3, 45.7, 44.2, 43.7, 42.1, 41.4, 38.6, 38.4, 36.9, 36.7, 36.3, 32.3, 32.1, 29.5, 28.0, 27.1, 25.5, 24.1, 22.6, 19.6, 13.5. Anal. Calcd for C₂₆H₄₄O₃: C, 77.18; H, 10.96. Found: C, 76.90; H, 10.99.

### 23(S)-Methyl-3,7-diketo-5β-cholan-24-oic acid (27)

To a suspension of 8 (0.2 g, 0.49 mmol) and silica gel (4 g, 200-400 mesh, Aldrich) in anhydrous CHCl₃ (2 mL) was added, portionwise, pyridinium chlorochromate (PCC, 0.61 g, 2.8 mmol) in 10 mL of CH₂Cl₂ and the reaction mixture was stirred at room temperature for 36 h. The mixture was filtered and the filtrate was washed with water (20 mL) and brine (20 mL). The organic layer was dried over Na₂SO₄ and concentrated. The crude product was purified by flash column chromatography (CH₂Cl₂/MeOH 9:1) to give the desired product as a white solid (0.15 g, 76% yield), mp: 118.6° C. ¹H NMR (CDCl₃) δ 3.62 (s, 3H), 2.84 (m, 1H), 2.46 (t, 2H), 1.23 (d, 3H), 0.90 (d, 3H), 0.67 (s, 3H). ¹³C NMR (CDCl₃) δ 212.5, 211.6, 184.1, 56.9, 50.9, 42.5, 41.5, 40.8, 39.6, 39.2, 37.1, 35.2, 34.9, 34.6, 34.5, 32.7, 30.1, 28.1, 23.5, 22.6, 20.5, 18.8, 18.1, 13.4. Anal. Calcd for C₂₅H₃₈O₄.½H₂O: C, 72.95; H, 9.31. Found: C, 72.36; H, 9.62.

Cell Culture

HEK293 and HEK293-TGR5 overexpressing cells were cultured in high glucose Dulbecco's modified Eagle's medium (DMEM, Cellgro, Manassas, Va.) with L-glutamine supplemented with 10% (vol/vol) heat-inactivated fetal bovine serum (Gemini Bio-Products, West Sacramento, Calif.). TGR5-overexpressing HEK293 cells were maintained in G418-containing media until plating. Cells were plated in 24-well plates (5×10⁵ cells/well) 24 h before transfection. Prior to transfection, cells were rinsed with PBS, and media was replaced with DMEM without phenol red supplemented with 10% super-stripped FBS.

TGR5 and FXR Luciferase Assay To evaluate TGR5 activity of compounds, cells were transfected 100 ng pCRE-luc reporter along with pCMV-β-galactosidase (10 ng) as an internal control for normalization of transfection efficiency. Plasmids were complexed with 2 mL of Fugene 6 reagent (Promega, Madison, Wis.) in OptiMEM (Invitrogen, Carlsbad, Calif.) and cells were transfected for 18 h. The following day, cells were treated with vehicle and appropriate ligand as indicated. Luciferase and β-galactosidase activities were assayed 6 h later using Luciferase Assay System (Promega) and Galacto-Star (Applied Biosystems, Foster City, Calif.) reagents, respectively, and a MLX luminometer (Dynex Technologies, Chantilly, Va.).

To evaluate the FXR activity of compounds, HEK293 cells were transfected with 25 ng of farnesoid X receptor expression plasmid (pCMX-hFXR), 25 ng of retinoid X receptor expression plasmid (pCMXhRXR), 100 ng of reporter plasmid (pEcREx6-TK-Luc), and 10 ng of pCMV-β-galactosidase as an internal control in each well, using Fugene 6 reagent. Approximately 18 h after transfection, cells were incubated for 12 h with different concentrations of each compound in DMEM without phenol red supplemented with 10% super-stripped FBS. Cells were lysed and normalized and luciferase activity was determined.

cAMP Assays.

HEK293 overexpressing TGR5 were treated with vehicle and appropriate ligand for 30 min in induction buffer comprised of serum-free Krebs Ringer buffer supplemented with 100 mM Ro 20-1274 and 500 mM IBMX (Sigma, St. Louis, Mo.) and cAMP levels were determined in lysates using cAMP-Glo Assay Kit (Promega) according to the manufacturer's protocol. Data analysis to determine EC₅₀ values was performed with GraphPad Prism software using a cAMP standard curve and sigmoidal dose-response (variable slope) equation based on dose-response curves with tested compounds.

### V. References

1. Wild, S.; Roglic, G.; Green, A.; Sicree, R.; King, H. Global prevalence of diabetes: estimates for the year 2000 and projections for 2030. Diabetes Care 2004, 27, 1047-53.
2. Wang, H.; Chen, J.; Hollister, K.; Sower, L. C.; Forman, B. M. Endogenous bile acids are ligands for the nuclear receptor FXR/BAR. Mol. Cell 1999, 3, 543-553.
3. Forman, B. M.; Goode, E.; Chen, J.; Oro, A. E.; Bradley, D. J.; Perlmann, T.; Noonan, D. J.; Burka, L. T.; McMorris, T.; Lamph, W. W.; Evans, R. M.; Weinberger, C. Cell. Identification of a nuclear receptor that is activated by farnesol metabolites. 1995, 81, 687-693.
4. Maruyama, T.; Miyamoto, Y.; Nakamura, T.; Tamai, Y.; Okada, H.; Sugiyama, E.; Nakamura, T.; Itadani, H.; Tanaka, K. Identification of membrane-type receptor for bile acids (M-BAR). Biochem. Biophys. Res. Commun. 2002, 298, 714-719.
5. Kawamata, Y.; Fujii, R.; Hosoya, M.; Harada, M.; Yoshida, H.; Miwa, M.; Fukusumi, S.; Habata, Y.; Itoh, T.; Shintani, Y.; Hinuma, S.; Fujisawa, Y.; Fujino, M. A G-protein-coupled receptor responsive to bile acids. J. Biol. Chem. 2003, 278, 9435-9440.
6. Pellicciari, R.; Sato, H.; Gioiello, A.; Costantino, G.; Macchiarulo, A.; Sadeghpour, B. M.; Giorgi, G.; Schoonjans, K.; Auwerx, J. Nongenomic actions of bile acids. Synthesis and preliminary characterization of 23- and 6,23-alkyl-substituted bile acid derivatives as selective modulators for the G-protein coupled receptor TGR5. J. Med. Chem. 2007, 50, 4265-4268.
7. Pellicciari, R.; Gioiello, A.; Macchiarulo, A.; Thomas, C.; Rosatelli, E.; Natalini, B.; Sardella, R.; Pruzanski, M.; Roda, A.; Pastorini, E.; Schoonjans, K.; Auwerx, J. Discovery of 6α-ethyl-23(S)-methylcholic acid (S-EMCA, INT-777) as a potent and selective agonist for the TGR5 receptor, a novel target for diabesity. J. Med. Chem. 2009, 52, 7958-7961.
8. Pellicciari, R.; Gioiello, A.; Sabbatini, P.; Venturoni, F.; Nuti, R.; Colliva, C.; Rizzo, G.; Adorini, L.; Pruzanski, M.; Roda, A.; Macchiarulo, A. Avicholic Acid: A Lead Compound from Birds on the Route to Potent TGR5 Modulators. ACS Med. Chem. Lett. 2012, 3, 273-277.
9. Iguchi, Y; Nishimaki-Mogami, T; Yamaguchi, M; Teraoka, F; Kaneko, T; Une, M. Effects of Chemical Modification of Ursodeoxycholic Acid on TGR5 Activation Biol. Pharm. Bull. 2011, 34, 1-7.
10. Campana, G.; Pasini, P.; Roda, A.; Spampinato, S. Regulation of ileal bile acid-binding protein expression in Caco-2 cells by ursodeoxycholic acid: Role of the farnesoid X receptor, Biochemical Pharmacology, 2005, 61, 1755-1763.
11. Thomas, C.; Pellicciari, R.; Pruzanski, M.; Auwerx, J.; Schoonjans, K. Targeting bile-acid signalling for metabolic diseases, Nature Reviews Drug Discovery 7, 2008, 678-693.
12. Sato, H.; Macchiarulo, A.; Thomas, C.; Gioiello, A.; Une, M.; Hofmann, A. F.; Saladin, R.; Schoonjans, K.; Pellicciari, R.; Auwerx, J. Novel potent and selective bile acid derivatives as TGR5 agonists: biological screening, structure-activity relationships, and molecular modeling studies. J. Med. Chem. 2008, 51, 1831-1841.
13. Kopka, I. E.; Lin, L. S.; Jewell, J. P.; Lanza, T. J.; Fong, T. M.; Shen, C. P.; Lao, Z. J.; Ha, S.; Castonguay, L. G.; Ploeg, L. Van der.; Goulet, M. T.; Hagmann, W. K. Synthesis and cannabinoid-1 receptor binding affinity of conformationally constrained analogs of taranabant, Bioorg. Med. Chem. Lett. 2010, 20, 4757-4761.
14. Yu, D.; Mattern, D. L.; Forman B. M. An Improved Synthesis of 6α-Ethylchenodeoxycholic Acid (6ECDCA), a Potent and Selective Agonist for the Farnesoid X Receptor (FXR), Steroids, 2012, 77, 1335-1338.
15. Miyashita, N; Yoshkoshi, A; Grieco, P. A. Pyridinium p-Toluenesulfonate. A mild and Efficient Catalyst for the Tetrahydropyranylation of Alcohols. J. Org. Chem. 1977, 426, 3772-3774.
16. Huang, M. A sample modification of the Wolff-Kishner reduction. J. Am. Chem. Soc. 1946, 68, 2487-2488.
17. ES 489,661, 1980.
18. ES 499,525, 1981.
19. U.S. Pat. No. 0,039,435 A1, 2011.
20. Bhattacharyya, P. K.; Bankawala, Y. G. Determination of chenodeoxycholic acid and ursodeoxycholic acid by nuclear magnetic resonance spectrometry. Analytical Chem., 1978, 50, 1462-1465.
21. Dixon, R. W.; Radmer, R. J.; Kuhn, B.; Kollman, P. A. Theoretical and Experimental Studies of Biotin Analogues That Bind Almost as Tightly to Streptavidin as Biotin, J. Org. Chem. 2002, 67, 1827-1837.
22. Beak, P; Meyers, A. I. Stereo- and regiocontrol by complex induced proximity effects: reactions of organolithium compounds, Acc. Chem. Res. 1986, 19, 356-363.
23. (a) Meerwein, H.; Schmidt, R. Halogenated alcohols. Justus Liebigs Ann. Chem. 1925, 444, 221. (b) Ponndorf, W. The reversible exchange of oxygen between aldehydes or ketones on the one hand and primary or secondary alcohols on the other hand. Angew. Chem. 1926, 39, 138. (c) Verley, M. Bull. Soc. Chim. Fr. 1925, 37, 871.
24. Hach, V. Meerwein-Ponndorf-Verley reduction of mono- and bicyclic ketones. Rate of reaction. J. Org. Chem. 1973, 38, 293-299.
25. Cohen, R.; Graves, C. R.; Nguyen, S. T.; Martin, J. M.; Ratner, M. A. The Mechanism of Aluminum-Catalyzed Meerwein-Schmidt-Ponndorf-Verley Reduction of Carbonyls to Alcohols. J. Am. Chem. Soc. 2004, 126, 14796-14803.
26. Yin, J.; Huffman, M. A.; Conrad, K. M.; Armstrong, J. D. Highly Diastereoselective Catalytic Meerwein-Ponndorf-Verley Reductions. J. Org. Chem. 2006, 71, 840-843.
27. Bhattacharya, S., Hall, S. E. and Vaidehi N., Agonist induced conformational changes in bovine rhodopsin: Insight into activation of G-protein coupled receptors, J. Mol. Biol. 2008, 382, 539-555.
28. Maestro, version 9.3, Schrödinger, LLC, New York, N.Y., 2012.
29. Glide, version 5.5, Schrödinger, Inc., New York, N.Y., 2009.
30. Steroids, 12^{th} Edition, p-65.

1. A compound having the formula: 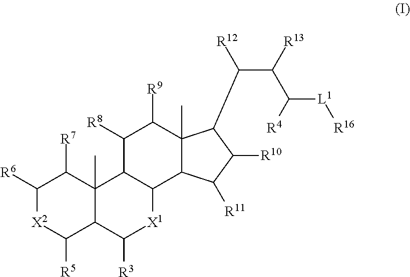
wherein,
L¹ is —C(O)—, —C(O)O—, —C(O)NH—, or —CH₂—;
X¹ is —C(O) or —C(R)(R²);
X² is —C(O) or —C(R¹⁴)(R⁵);
R¹ is hydrogen, unsubstituted alkyl, or —OR^{1A};
R² is hydrogen, unsubstituted alkyl, or —OR^{2A};
R³ is hydrogen, unsubstituted alkyl, or —OR^{3A};
R⁴ is hydrogen or unsubstituted alkyl;
R⁵ is hydrogen, unsubstituted alkyl, or —OR^{5A};
R⁶ is hydrogen, unsubstituted alkyl, or —OR^{6A};
R⁷ is hydrogen, unsubstituted alkyl, or —OR^{7A};
R⁸ is hydrogen, unsubstituted alkyl, or —OR^{8A};
R⁹ is hydrogen, unsubstituted alkyl, or —OR^{9A};
R¹⁰ is hydrogen, unsubstituted alkyl, or —OR^{10A};
R¹¹ is hydrogen, unsubstituted alkyl, or —OR^{1A};
R¹² is hydrogen, unsubstituted alkyl, or —OR^{12A};
R¹³ is hydrogen, unsubstituted alkyl, or —OR^{13A};
R¹⁴ is hydrogen, unsubstituted alkyl, or —OR^{14A};
R¹⁵ is hydrogen, unsubstituted alkyl, or —OR^{15A};
R¹⁶ is hydrogen, halogen, —N₃, —CF₃, —CCl₃, —CBr₃, —CI₃, —CN, —CHO, —OR^{16A}, —NHR^{16A}, —COOH, —CONH₂, —NO₂, —SH, —SO₂Cl, —SO₃H, —SO₄H, —SO₂NH₂, —NHNH₂, —ONH₂, —NHC(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, or a carboxylate protecting group;
R^{1A}, R^{2A}, R^{3A}, R^{5A}, R^{6A}, R^{7A}, R^{8A}, R^{9A}, R^{10A}, R^{11A}, R^{12A}, R^{13A}, R^{14A}, R^{15A} are independently hydrogen, unsubstituted alkyl, or an alcohol protecting group;
R^{16A} is hydrogen, halogen, —N₃, —CF₃, —CCl₃, —CBr₃, —CI₃, —CN, —CHO, —OH, —NH₂, —COOH, —CONH₂, —NO₂, —SH, —SO₂Cl, —SO₃H, —SO₄H, —SO₂NH₂, —NHNH₂, —ONH₂, —NHC(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, an alcohol protecting group, or a carboxylate protecting group;
wherein, if X¹ or X² is —C(H)OH and R¹, R², R³, and R⁴ are hydrogen, then -L¹-R¹⁶ is not —C(O)OH; and
wherein, if X¹ and X² are —C(H)OH, R³ is α-ethyl and R⁴ is hydrogen, then -L¹-R¹⁶ is not —C(O)OH.

2. The compound of claim 1, wherein R^{1A}, R^{2A}, R^{3A}, R^{5A}, R^{6A}, R^{7A}, R^{8A}, R^{9A}, R^{10A}, R^{11A}, R^{12A}, R^{13A}, R^{14A}, R^{15A}, and R^{16A} are hydrogen.

3. The compound of claim 1, wherein R⁵, R⁶, R⁷, R⁸, R⁹, R¹⁰, R¹¹ and R¹³ are hydrogen.

4. The compound of claim 3, wherein L¹ is —C(O)—, —C(O)O—, or —C(O)NH—.

5. The compound of claim 4, wherein R⁴ is unsubstituted alkyl.

6. The compound claim 5, wherein R⁴ is attached to a chiral carbon having an (S) stereochemistry.

7. The compound of claim 6, wherein
X¹ is —C(R¹)(R²); and
X² is —C(R¹⁴)(R¹⁵).

8. The compound of claim 7, wherein
R¹ is —OR^{1A}; and
R¹⁴ is —OR^{14A}.

9. The compound of claim 8, wherein
L¹ is —C(O)— or —C(O)NH—; and
R¹⁶ is substituted or unsubstituted alkyl or substituted or unsubstituted heteroalkyl.

10. The compound of claim 8, wherein
L¹ is —C(O)O—; and
R¹⁶ is hydrogen, or a carboxylate protecting group.

11. The compound of claim 6, wherein
R¹ and R² are hydrogen; and
R¹⁴ is OR^{14A}.

12. The compound of claim 11, wherein
L¹ is —C(O)— or —C(O)NH—; and
R¹⁶ is substituted or unsubstituted alkyl or substituted or unsubstituted heteroalkyl.

13. The compound of claim 11, wherein
L¹ is —C(O)O—; and
R¹⁶ is hydrogen, or a carboxylate protecting group.

14. The compound of claim 6, wherein
X¹ is C(O); and
X² is —C(O) or —C(H)(OH).

15. The compound of claim 14, wherein
L¹ is —C(O)— or —C(O)NH—; and
R¹⁶ is substituted or unsubstituted alkyl or substituted or unsubstituted heteroalkyl.

16. The compound of claim 14, wherein
L¹ is —C(O)O—; and
R¹⁶ is hydrogen, or a carboxylate protecting group.

17. The compound of claim 1 having formula: 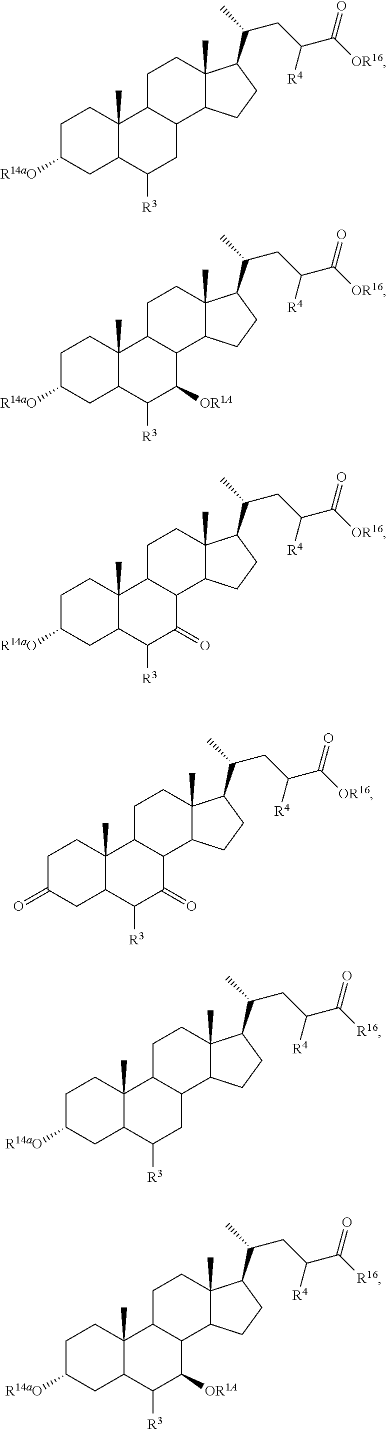 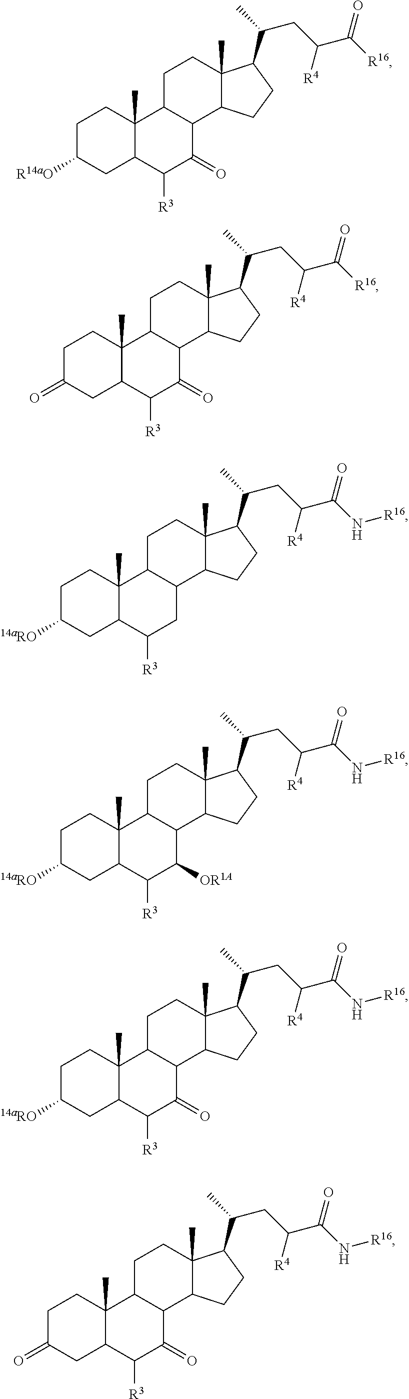
or a pharmaceutically acceptable salt thereof.

18. The compound of any one of claim 17, wherein R³ is ethyl.

19. The compound of any one of claim 18, wherein R³ is attached to a chiral carbon having an (S) stereochemistry.

20. The compound of claim 19, wherein R⁴ is unsubstituted alkyl.

21. The compound of claim 1, having formula: 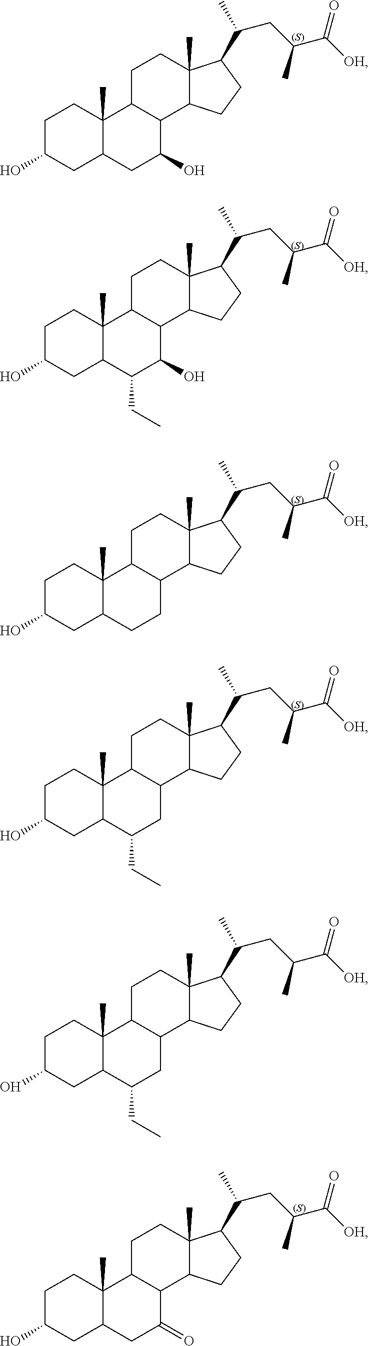 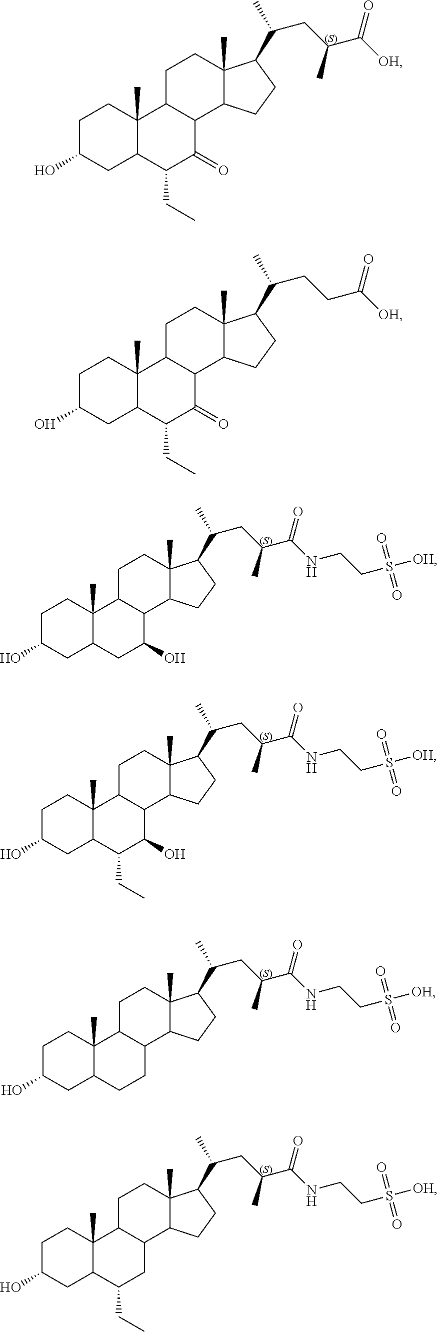 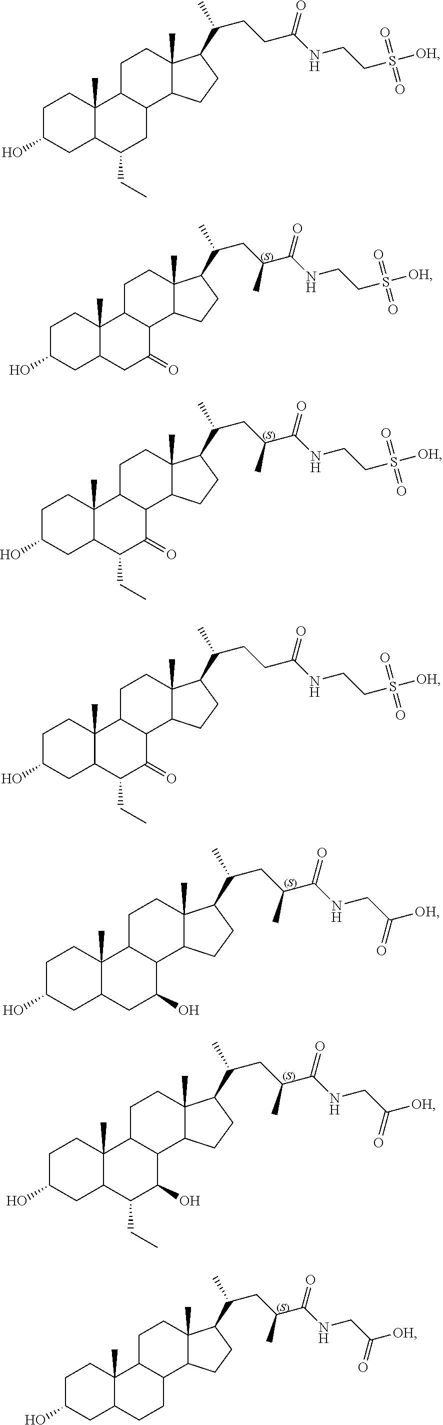 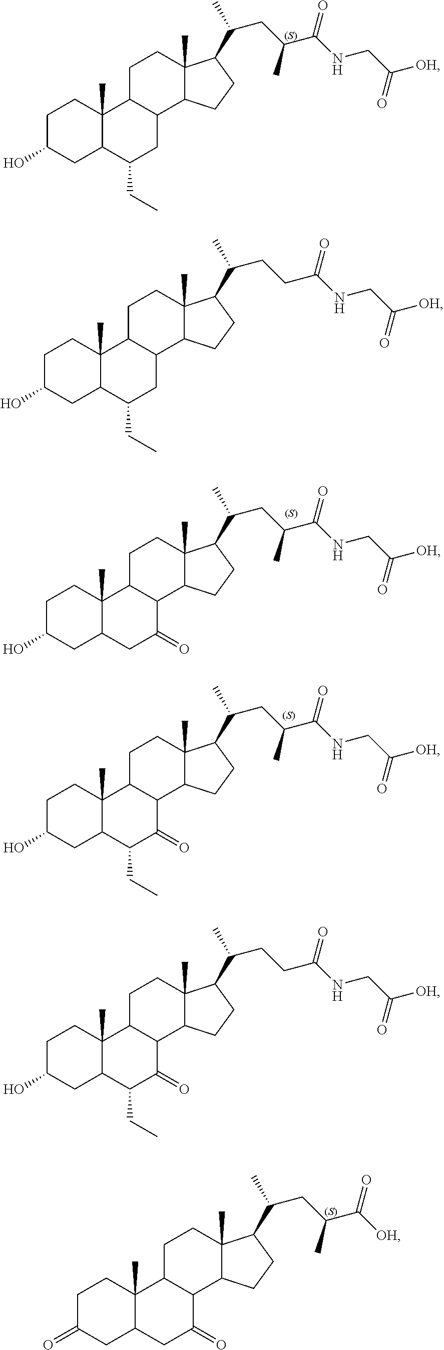 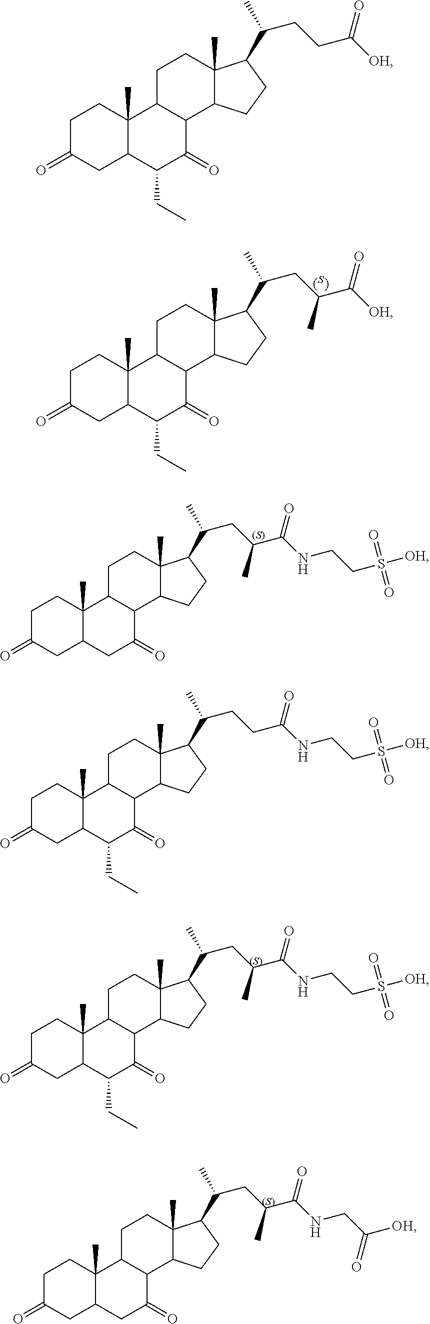 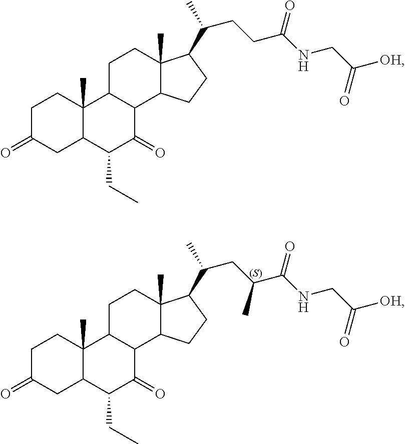
or a pharmaceutically acceptable salt thereof.

22. The compound of claim 1 having formula: 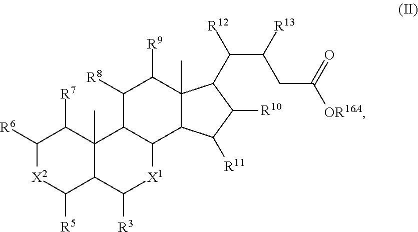
wherein R^{16A} is a carboxylate protecting group.

23. A pharmaceutical composition comprising a pharmaceutically acceptable excipient and a compound of claim 1.

24. A method of synthesizing a compound of Formula (I) of claim 1, the method comprising:
(i) contacting a compound of Formula (II) of claim 22 with an alkylating agent in the presence of a sterically hindered base; and
(ii) contacting said compound of Formula (II) of claim 22 with a carboxylate deprotecting agent, thereby synthesizing a compound of Formula (I) of claim Error! Reference source not found.

25. The method of claim 24, wherein one or more of R¹, R², R³, R⁵, R⁶, R⁷, R⁸, R⁹, R¹⁰, R¹¹, R¹², R¹³, R¹⁴, and R¹⁵ of the compound of formula (II) is respectively —OR^{1A}, —OR^{2A}, —OR^{3A}, —OR^{5A}, —OR^{6A}, —OR^{7A}, —OR^{8A}, —OR^{9A}, —OR^{10A}, —OR^{11A}, —OR^{12A}, —OR^{13A}, —OR^{14A}, —OR^{15A}, and wherein R^{1A}, R^{2A}, R^{3A}, R^{5A}, R^{6A}, R^{7A}, R^{8A}, R^{9A}, R^{10A}, R^{11A}, R^{12A}, R^{13A}, R^{14A} and R^{15A} are independently an alcohol protecting group.

26. The method of claim 25, wherein the method further comprises contacting said compound with an alcohol deprotecting agent.

27. The method of claim 24, wherein X¹ is —C(O).

28. The method of claim 27, further comprising contacting X¹ of the compound of Formula (II) with a reducing agent to form a —CH₂ or —C(H)OH.

29. The method of claim 24, wherein said alkylation agent is an alkyl halide.

30. The method of claim 24, wherein said sterically hindered base is (M⁺¹)HMDS, (M⁺)tBuO, (M⁺¹)TMP, (M⁺¹)PhO, (M⁺¹)MeO, (M⁺¹)EtO, DBU, Dabco, N,N-dichlorohexylmethylamine, N,N-diisopropyl-2-ethylbutylamine, 2,6-di-tert-butyl-4-methylpyridine, pentamethylpiperidine, MTBD, PMDBD, TBD, or tri-tert-butylpyridine, wherein (M⁺¹) is Na, K, or Li.

31. The method of claim 30, wherein said sterically hindered base is (M⁺¹)HMDS, wherein (M⁺¹) is Na, K, or Li.

32. The method of claim 24, wherein said contacting is performed in the presence of a polar aprotic solvent.

33. The method of claim 32, wherein said polar aprotic solvent is HMPA, HMPT, DMF, DMSO, MeCN, dioxane, methylpyrrolidone, DMPU, or a tetra-alkyl urea.

34. A method of treating or preventing diabetes, obesity, insulin resistance, or liver disease in a subject in need thereof, said method comprising administering to said subject a therapeutically effective amount of the compound of claim 1.

35. The method of claim 24, wherein said subject is administered said compound for treating or preventing diabetes.

36. The method of claim 35, wherein said diabetes is type 2 (T2D) diabetes.

37. The method of claim 34, wherein said subject is administered said compound for treating or preventing obesity.

38. The method of claim 34, wherein said subject is administered said compound for treating or preventing insulin resistance.

39. A method of treating or preventing cancer, said method comprising administering to said subject a therapeutically effective amount of the compound of claim 1.

40. The method of claim 39, wherein said cancer is liver cancer.
